(19) United States
(12) Patent Application Publication
(10) Pub. No.: US 2024/0130957 A1
(43) Pub. Date: Apr. 25, 2024

(54) EXTRACTS OF ISOCHRYSIS SPECIES/TISOCHRYSIS SPECIES

(71) Applicant: SYMRISE AG, Holzminden (DE)

(72) Inventors: Martina HERRMANN, Hartina (DE); Dominik STUHLMANN, Holzminden (DE); Sandra GAEBLER, Höxter (DE); Nicole TITZE, Holzminden (DE); Ann-christin WESELOH, Polle (DE); Ricarda KRÂLING, Holzminden (DE)

(21) Appl. No.: 18/266,362

(22) PCT Filed: Dec. 14, 2021
(86) PCT No.: PCT/EP2021/085773

§ 371 Date: Jun. 9, 2023

(30) Foreign Application Priority Data
Dec. 14, 2020 (EP) 20213821.0
Jan. 27, 2021 (WO) PCT/EP2021/051840

(51) Int. Cl. A61K 8/9711; A61K 8/31; A61K 8/35; A61K 8/36; A61K 8/49; A61K 8/60; A61K 36/03; A61P 17/16; A61P 17/18; A61Q 17/00; A61Q 19/04

(52) U.S. Cl. CPC A61K 8/9711; A61K 8/31; A61K 8/35; A61K 8/361; A61K 8/494; A61K 8/60; A61K 36/03; A61P 17/16; A61P 17/18; A61Q 17/00; A61Q 19/04; A61K 2236/37; A61K 2800/58; A61K 2800/80

(57) ABSTRACT

Suggested is a supercritical fluid CO2 extract of Isochrysis sp. and/or Tisochrysis sp. comprising: a) not more than 1.5% b.w. of carotenoids; b) not more than 2.0% b.w. of chlorophylls; and c) at least 15% b.w. of free fatty acids, each weight percentage amount based on the total weight of the extract, wherein the extract has a gardner color value of not more than 9 at an extract concentration of 0.018% in oil.

### AREA OF INVENTION

The present invention relates to extracts of Isochrysis sp., preferably Tahitian Isochrysis, its cosmetic, dermatological and/or therapeutic uses and compositions and cosmetic, dermatological or therapeutic products comprising such an extract of Isochrysis sp., preferably Tahitian Isochrysis.

### BACKGROUND OF THE INVENTION

Tanned skin is still a beauty ideal in certain regions of the world, especially in Western areas such as Northern American and European countries. Since it is well-known that natural and artificial ultraviolet radiation can have severe skin and health consequences (e.g. photoaging and skin cancer), sunless tanning products are valuable substitutes to sunbathing and indoor tanning and thus an alternative way to achieve a tanned skin. Also there exists the wish to accelerate, prolong and/or intensify the natural tan which can also be achieved by application of sunless tanning products.

Artificial skin tanning or browning can be carried out cosmetically or medically, the following main approaches playing a role:

If carotene preparations are taken regularly or fruits and vegetables high in carotene content are excessively consumed, cutaneous carotene levels increase and the skin gradually turns orange to yellow-orange (carotenemia). However, skin tone looks different to natural skin tan.

Furthermore, washable makeup preparations can be used to achieve a light skin tinting (e.g. extracts of fresh green walnut shells, henna).

Skin browning can also be achieved by chemical changes to the skin's stratum corneum using so-called self-tanning preparations. The most important active ingredient is dihydroxyacetone (CAS number 96-26-4), a 3-carbon sugar allowed by the Food and Drug Administration (FDA) as a color additive in sunless tanning products. Dihydroxyacetone is used worldwide in cosmetic products, i.e. skin care products for face and body, instant tan formulations and ‘flash bronzers’ in combination with colorants. The recommended use levels depend on the skin type and tanning status of the user. SCCS/1347/10 (Scientific Committee on Consumer Safety opinion on dihydroxyacetone by) give following typical use levels: dihydroxyacetone is used as a self-tanning agent in leave-on cosmetic products up to 10%. In addition, dihydroxyacetone is also reported to be used in spray cabins in aqueous solutions in concentrations between 8 and 14%. A Faurschou and H C Wulf (Photodermatol Photoimmunol Photomed., 2004, 20(5), 239-42) even describe the application of a 20% dihydroxyacetone cream twice per day on the volar forearm of 10 volunteers with light skin types (types II-III) for 7 days.

The skin browning achieved in this way does not wash off and is only removed with the normal flaking of the skin (after around 5 to 10 days). Dihydroxyacetone can be classed as a ketotriose and as a reducing sugar it reacts with the amino acids and amino groups of proteins present in sweat and in the skin such as glycine, alanine, leucine, and valine or the free amino and imino groups in keratin via a series of intermediate steps along the lines of a Maillard reaction to form brown-coloured substances known as melanoids (melanin-mimetic cutaneous pigments), which are occasionally also called melanoidins or melanoidins.

One disadvantage of this is that unlike “sun-tanned” skin, the skin browning obtained with dihydroxyacetone does not protect the skin against sunburn. A further disadvantage of dihydroxyacetone lies in the fact that, particularly under the influence of ultraviolet radiation, it releases formaldehyde, albeit usually in small amounts. Dihydroxyacetone also has an unpleasant, chemical odour.

Furthermore, R. V. Lloyd et al., described the detection of ex vivo and in vivo formation of Maillard reaction free radicals in mouse skin after application of dihydroxyacetone dissolved in buffer by electron spin resonance (J. Invest. Dermatol., 2001, 117 (3), 740-742). Investigation of the cutaneous effects of acute dihydroxyacetone exposure of cultured human HaCaT keratinocytes by J. Perer et al. (Redox Biology 2020, 36, 101594) showed that dihydroxyacetone up to 20 mM did not impair viability but caused a pronounced cellular stress response. Likewise, a cosmetic use-relevant topical dihydroxyacetone dose-regimen elicited a pronounced transcriptional stress response observable in human epidermal reconstructs. For comparison, stress response gene expression array analysis was performed in epidermis exposed to a supra-erythema) dose of solar simulated UV (2 MEDs), identifying genes equally or differentially sensitive to either one of these cutaneous stimuli [dihydroxyacetone (‘sunless tanning’) versus solar UV (‘sun-induced tanning’)]. Additionally, the reaction of the reducing sugars used in self-tanning products and amino acids in the skin layer (Maillard reaction) leads to the formation of Amadori products that generate free radicals during UV irradiation. Using the electron spin resonance spectroscopy method (K. Jung et al., Spectrochim Acta A Mol Biomol Spectrosc. 2008, 69, 1423-1428) three different self-tanning agents were analyzed and it was found, that in dihydroxyacetone-treated skin more than 180% additional radicals were generated during sun exposure with respect to untreated skin. For this reason, the exposure duration in the sun must be shortened when self-tanners are used and photoaging processes are accelerated.

D-Erythrulose (also known as erythrulose, CAS number 40031-31-0) is a tetrose carbohydrate which is used in self-tanning cosmetics, often combined with dihydroxyacetone or other reducing sugars. Erythrulose reacts in much the same way on the skin surface as dihydroxyacetone, but it produces a lighter and slower-developing tan. When used alone, it fades faster than a dihydroxyacetone-based sunless tan. Erythrulose, however, has also been shown to increase production of free radicals similar to the effect seen with dihydroxyacetone (K. Jung et al., Spectrochim Acta A Mol Biomol Spectrosc. 2008, 69, 1423-1428).

The tint obtained with self-tanning agents is achieved without exposure to sunlight. In contrast, so-called “pre-tan products” or “tan promoters” are also available, which have to be applied before exposure to sunlight. In the sun these preparations then turn yellow, giving rise to a light brown-yellow colouring of the epidermis which further boosts the “suntan”.

Another type of artificial browning which is not dependent on UV light can be brought about through the hormones which are usually also released in the body as a consequence of (natural) UV irradiation and ultimately stimulate the melanocytes to synthesize melanin. Examples which can be cited in this connection are derivatives of proopiomelanocortin (POMC) such as [alpha]-MSH (Melanocyte Stimulating Hormone) and synthetic variants (such as [NIe(4), D-Phe(7)]-[alpha]-MSH), which in some cases display far higher activity levels than the natural [alpha]-MSH. Although these hormones can cause browning in principle, their use in cosmetics is prohibited, since they are pharmacologically potent substances (hormones) which should not be widely used without medical indications.

There are also known biologically acting skin tanning agents which stimulate the melanogenesis. Contrary to dihydroxyacetone the skin tan gained with biologically acting tanners consists of melanin and therefore has natural sun protection qualities and remains in the skin for a much longer time.

Acetyl tyrosine is a natural amino acid bound to acetic acid and provides the substrate for the generation of melanin synthesized along the lines of the physiologic pathways. Many tanning products that stimulate the synthesis of melanin in the skin contain acetyl tyrosine alone or in combination (e.g. in products: MelanoBronze, a combination of acetyl tyrosine and extract of the Monk's pepper, INCI: Vitex Agnus Castus Extract, Acetyl Tyrosine, Glycerin, Alcohol, Aqua, Suntan Accelerator PSP, INCI: Glycerin, Aqua, Acetyl Tyrosine, Riboflavin, Phenoxyethanol, Potassium Sorbate and Unipertan™ P-2002, INCI: Acetyl Tyrosine, Adenosine Triphosphate, Hydrolyzed Collagen, Riboflavin.

Other biologically acting tanners are e.g. D-chiro-inositol, a natural ingredient processed e.g. from the Carob tree, dihydroxy methylchromonyl palmitate or green pea extract (trade name Helostatine IS). These actives also intensify and maintain a suntan longer.

EP 2 168 570 describes extracts of Isochrysis sp., preferably Tahitian Isochrysis, for influencing or modifying growth of human hair or pigmentation of human skin and/or hair. Use of water as extractant provides extracts which increase stimulation of pigmentation of human skin/and or hair without stimulating hair growth. Extracts which stimulate the pigmentation of human skin and/or hair and growth of human hair are obtained by use of ethyl acetate or a mixture of hexane/ethyl acetate as extractant. The extraction process comprises extracting cell material of Isochrysis sp., preferably Tahitian Isochrysis for up to 24 hours at a temperature of not more than 50° C., preferably at a temperature of 16-40° C. and most preferably at a temperature of 20-30° C.

The product described in EP 2 168 570 was launched several years ago as skin tanner, however, the intensive color as well as color instability especially in cosmetic formulations containing the product are not well acceptable by the consumer and require special actions to slow down discoloration as far as possible. As UV-independent biological skin tanning and prolongation of obtained sun tan by stimulation of cutaneous melanin synthesis is still a big customer demand, there is the need to provide products with significantly reduced color and thereby much easier formulation properties while keeping at least comparable biological efficacy.

M. Herrmann et al. described an ethyl acetate extract obtained by extraction of freeze-dried Isochrysis sp., Tahitian strain (T-Iso) biomass for 16 h at room temperature protected from light giving an average extraction yield: 20% as a novel skin tanner (conference paper presented at 27th IFSCC congress, 2012). The dry extract increased cutaneous melanin at 0.2 and 2 ppm and was characterized by 25-29% fatty acids, 10-13% of glycerol bound fatty acids, 30-36% C37/38 alkenones, 4.7-5.5% chlorophyll, 2.4-3.5% carotenoids and 0.25-0.27% proteins. However, the chlorophyll and carotenoids content is high, which leads to an unpleasant color of the product and formulations containing it as well as light-induced discoloration due to degradation of these light sensitive pigments.

The use of SFE to extract microalgae was described e.g. in the review of I. Michalak et al. (J Chem 2015, Article ID 597140). In the presented examples, supercritical CO2 was chosen as solvent, occasionally supported by ethanol as modifier and operational conditions ranged within 40-85° C. and 78.6-500 bar. Depending on the applied conditions, SFE was used to extract e.g. pigments, lipids, polyphenols, and vitamins.

Total lipids generally cover neutral lipids, glycolipids and phospholipids, all known to occur in microalgae and in Isochrysis galbana (D. L. Alonso et al., Phytochem. 1998, 47, 1473-1481). Studies on extraction of oil seeds showed that a general feature of fatty oils extracted by supercritical CO2 is presence of phospholipids and glycolipids only in traces or not at all (E. Stahl et al., Dense Gases for Extraction and Refining, Springer 1988, p. 94).

SFE of freeze-dried Isochrysis galbana (T-ISO) was already described by Gilbert-Lopez et al. (Green Chem. 2015, 17, 4599-4609). Goal of the SFE with CO2 was to maximize the extraction yield and carotenoid content, while minimizing chlorophylls. SFE was performed at 100, 200 and 300 bar and temperatures of 40, 50 and 60° C. and optimum SFE conditions determined by use of a response surface methodology were 299 bar and 51° C. giving an extraction yield of 4.41%, a total carotenoid content of 16.4 mg carotenoids per g extract (1.64%) and a total chlorophyll content of 4.3 mg chlorophylls per g extract (0.043%). SFE extracts were also found to be rich in triacylglycerides.

SFE of wet Isochrysis galbana biomass by CO2 to recover lipids (conditions between 250-400 bar and 40-70° C.) was described by E. Ibanez et al. (conference paper presented at 12th International Symposium on Supercritical Fluids, 2018). Before the SFE, the wet biomass was extracted in a first step by subcritical water (employing mainly the residual water contained in the wet biomass) at 10-100 bar and 30-50° C. to recover soluble proteins and in a second step using carbon dioxide expanded ethanol (CXE) (pressures between 50-100 bar, 40-60° C. and ethanol percentages 40-80%) to gain a pigment extract.

Therefore, there exists in the art an ongoing need to provide further safe and effective agents for stimulating and boosting skin and/or hair (meaning scalp hair, eye lashes, eye brows, beard or other hair) pigmentation. These should ideally be natural or producible in a sustainable way, stable, without intensive colour and/or odour as well as easy to handle and formulate in common applications. Moreover, they should give a natural tan, when applied to the skin and/or hair.

### DESCRIPTION OF THE INVENTION

According to the invention, there is thus provided a supercritical fluid CO2 extract of Isochrysis sp. and/or Tisochrysis sp. comprising
a) not more than 1.5% b.w. of carotenoids,
b) not more than 2.0% b.w. of chlorophylls, and
c) at least 15% b.w. of free fatty acids,
each weight percentage amount based on the total weight of the extract,
wherein the extract has a Gardner color value of not more than 9 at an extract concentration of 0.018% in oil.

Surprisingly it has been discovered that a supercritical fluid extract obtained from Isochrysis or Tisochrysis sp., preferably Isochrysis galbana, more preferably Tahitian Isochrysis (T-ISO, after 2013 renamed to Tisochrysis lutea) characterized by a total chlorophyll content of not more than 2.0% b.w., a total carotenoid content of not more than 1.5% b.w., and a total content of free fatty acids of at least 15% b.w. significantly increases the cutaneous melanin level. Furthermore, the extract according to the invention improves skin's and/or hair's well-being by reducing oxidative stress induced changes, increasing skin and/or hair hydration and improving cutaneous barrier properties. Furthermore, the extract according to the invention significantly reduces oxidative stress induced changes.

“Oxidative stress” according to the invention reflects an imbalance between the systemic manifestation of reactive oxygen species and a biological system's ability to readily detoxify the reactive intermediates or to repair the resulting damage. In a preferred embodiment according to the invention, the oxidative stress is UV-induced oxidative stress. In a further preferred embodiment according to the invention, wherein the oxidative stress is Maillard reaction induced oxidative stress, in other words, oxidative stress induced by dihydroxyacetone and/or erythrulose, which act via Maillard reaction. This is especially advantageous as a combination of fast acting self-tanners acting via Maillard reaction like dihydroxyacetone and/or erythrulose and the slower acting biological tanning extract of the invention provide a consumer expected visual tan in faster time than the extract of the invention alone.

The terms “% b.w.”, “wt. %” and “% by weight” all refer to weight percentages and are used interchangeably.

In a preferred embodiment according to the invention, the extract has not more than 2.0% b.w. of phospholipids, based on the total weight of the extract. In a further preferred embodiment according to the invention, the extract has not more than 2.0% b.w. of glycolipids, based on the total weight of the extract. Furthermore, the ash content of the extract is preferably not more than 0.5% b.w., based on the total weight of the extract.

Preferably, there is thus provided a supercritical fluid CO2 extract of Isochrysis sp. and/or Tisochrysis sp. comprising
a) not more than 1.5% b.w. of carotenoids,
b) not more than 2.0% b.w. of chlorophylls,
c) at least 15% b.w. of free fatty acids,
d) not more than 2.0% b.w. of phospholipids, and
e) not more than 2.0% b.w. of glycolipids,
each weight percentage amount based on the total weight of the extract,
wherein the extract has a Gardner color value of not more than 9 at an extract concentration of 0.018% in oil and an ash content of not more than 0.5% b.w., based on the total weight of the extract.

In a preferred embodiment according to the invention, the extract is obtained from Isochrysis galbana. In a more preferred embodiment according to the invention, the extract is obtained from Tahitian Isochrysis.

The Isochrysis sp., preferably Isochrysis galbana, more preferably Tahitian Isochrysis (T-ISO, after 2013 renamed to Tisochrysis lutea) extract according to the invention can be extracted of the dried biomass by means of supercritical fluid extraction (SFE) with carbon dioxide.

Isochrysis sp., representatives of the Isochrysidaceae, a family of non-calcifying organisms within the haptophyte order Isochrysidales, are widely used as a food source in aquaculture. They are marine microalgae rich in long chain polyunsaturated fatty acids (LC PUFAs) e.g. docosahexaenoic acid (DHA) and eicosapentaenoic acid (EPA), photosynthetic pigments i.e. chlorophylls and carotenoids (especially fucoxanthin), α-tocopherol, sterols such as sitosterol and stigmasterol, brassicasterol, carbohydrates, proteins as well as other nutrients.

Isochrysis sp. produce also lipids. Total lipids of Isochrysis galbana (D. L. Alonso et al., Phytochem. 1998, 47, 1473-1481) were characterized by 43% of neutral lipids (triacylglycerols, diacylglycerols, and monoacylglycerols), 37% of glycolipids (monogalactosylacylglycerols, digalactosylacylglycerols, and sulphoquinovose diacylglycerols) and 20% of phospholipids. Therefore, the extract of the present invention is particularly surprising since it preferably comprises not more than 2.0% b.w. of phospholipids, and not more than 2.0% b.w. of glycolipids, calculated on the total weight of the extract. This is advantageous, because phospholipids and glycolipids might negatively influence typical cosmetic formulations such as o/w and w/o formulations. Due to their amphiphilic nature, co-emulsifying and surfactant like properties they can lead to destabilization of a cosmetic formulation when the sum of emulsifying and surfactant like constituents in the formulation gets too high. The extract of the invention with not more than 2.0% b.w. of phospholipids, and not more than 2.0% b.w. of glycolipids thus allows easy incoperation in established cosmetic formulation without requiring adaption of the formulation due to its addition.

Isochrysis sp. also produce long-chain unsaturated methyl- or ethyl-ketones (I. T. Marlowe et al., Br. Phycol. J. 1984, 19, 203-216). In these species, C37-methyl-alken-2-ones, containing two to four trans-type carbon double bonds, are most prominent accompanied by C38-alken-3-ones. Among haptophyte lineages, members of the order Isochrysidales are unique in being the exclusive producers of long-chain ketones that are commonly used for paleotemperature reconstructions as the proportion of the triunsaturated C37 alkenone has been shown to increase with decreasing temperature of the water in which the microalgae g row.

Isochrysis sp. can be cultured readily, are small enough to be ingested by larval stages of invertebrates, are digestible, and are capable of supporting growth of a number of invertebrates of commercial value. Therefore, Isochrysis sp. are widely used in aquaculture already since decades and are commercially produced as feed for the early larval stages of mollusks, fish, and crustaceans. Isochrysis sp. are typically cultivated in photobioreactors and the biomass is typically harvest by common solid-liquid separation techniques e.g. filtration, sedimentation, flotation, and centrifugation. Dewatering or drying of the obtained microalgae biomass concentrate or paste can be performed by common technologies such as thermal drying, freeze drying, spray drying or vacuum drying and generates a dry solid with increased stability and better storage properties.

Isochrysis aff. galbana T-ISO (strain numbers from different culture collections=CCAP927/14, CCMP1234, CS-177 etc.) often also referred to as Tahitian Isochrysis is extensively used in aquaculture. The morphological and ultrastructural characters of T-Iso are extremely similar to those of Isochrysis galbana. Bendif and Probert proposed the erection of one new genus (Tisochrysis gen. nov.) and two new species (Tisochrysis lutea sp. nov. and Isochrysis nuda sp. nov.), because of the existing genetic separation of the Isochrysis and T-Iso clusters. The Tisochrysis Glade is composed of the strain informally known as Isochrysis aff. galbana T-ISO (“Tahiti isolate”), together with other genetically identical or quasi-identical strains from the Pacific and Atlantic ocean as well as from the North Sea (E. M. Bendif et al., J. Appl. Phycol. 2013, 25, 1763-1776).

Supercritical fluid extraction (SFE) technology for extraction of lipophilic bioactive natural compounds from microalgae has been employed from laboratory to the commercial scale. SFE utilizes supercritical fluids, which above their critical point exhibit liquid-like (solvent power, negligible surface tension) as well as gas-like (transport) properties. CO2 is the supercritical solvent of choice in the extraction of lipophilic compounds, since it is a chemically inert, odorless, colorless, highly pure, safe, cost effective, non-toxic, non-flammable and recyclable gas allowing supercritical operation at relatively low pressures (critical pressure 7.3 MPa=73 bar) and near room temperature (critical temperature 31.2° C.). Supercritical CO2 (SC CO2) behaves like a lipophilic solvent but, compared to liquid solvents, it has the advantage that its selectivity or solvent power is adjustable. The versatility of SC CO2 as extraction technology is linked to the possible drastic change of solvent power through the change of pressure and temperature. Therefore, SC CO2 is suitable for the extraction of many non-polar to moderately polar compounds, while more polar compounds can only be extracted by the use of SC CO2 and co-solvents. SFE can be considered a sound cleantech strategy to extract natural compounds with an undisputed environmental friendliness. This is due to the non-toxic nature of SF CO2. Furthermore, this extraction technology is available in industrial scale.

Moisture content of the raw material influences not only the extraction quality and yield but also the fluid dynamics of the solvent. The contained water can act as co-solvent by interacting with the supercritical CO2 and by changing the overall polarity of the fluid. Drying the raw material by known techniques such as e.g. freeze-drying, spray-drying, vacuum-drying is preferred according to the invention in order to have a water content of not more than 15%, more preferably not more than 12%, most preferably not more than 10%.

SFE utilizes high pressure and temperature, breaks algal cells and extract compounds. However, as microalgae have more or less rigid cell walls, pre-treatment with cell-disruption techniques can be advantageous as down-stream processes such as extraction or recovery of valuable constituents can be facilitated and improved thereby. Examples of existing cell disruption technologies include e.g. bead milling, grinding (cryogenic), high pressure homogenization, ultrasonication, microwave treatment, microfluidization, enzymatic disruption or a combination of them. Milling such as bead milling is a mechanical cell disruption method with preferred properties.

Fractional supercritical fluid extraction can be used to further enrich desired constituents and decrease or even remove undesired constituents such as e.g. volatiles, lipids, pigments, or waxes.

In a preferred embodiment according to the invention, the extract comprises not more than 1.0% b.w. of carotenoids, based on the total weight of the extract. In a further preferred embodiment according to the invention, the extract comprises not more than 0.5% b.w. of carotenoids, based on the total weight of the extract. In other words, the extract comprises preferably carotenoids in an amount from 0.000001 to 1.5% b.w., more preferably in an amount from 0.000001 to 1.0% b.w., more preferably from 0.000001 to 0.5% b.w., based on the total weight of the extract.

In a preferred embodiment according to the invention, the extract comprises not more than 1.0% b.w. of chlorophylls, based on the total weight of the extract. In a further preferred embodiment according to the invention, the extract comprises not more than 0.5% b.w. of chlorophylls, based on the total weight of the extract. In other words, the extract comprises preferably chlorophylls in an amount from 0.000001 to 2.0% b.w., more preferably in an amount from 0.000001 to 1.0% b.w., more preferably from 0.000001 to 0.5% b.w., based on the total weight of the extract.

In a preferred embodiment according to the invention, the extract comprises not more than 1.0% b.w. of phospholipids, based on the total weight of the extract. In a further preferred embodiment according to the invention, the extract comprises not more than 0.5% b.w. of phospholipids, based on the total weight of the extract. In other words, the extract comprises preferably phospholipids in an amount from 0.0000001 to 2.0% b.w., more preferably in an amount from 0.0000001 to 1.0% b.w., more preferably from 0.0000001 to 0.5% b.w., based on the total weight of the extract.

In a preferred embodiment according to the invention, the extract comprises not more than 1.0% b.w. of glycolipids, based on the total weight of the extract. In a further preferred embodiment according to the invention, the extract comprises not more than 0.5% b.w. of glycolipids, based on the total weight of the extract. In other words, the extract comprises preferably glycolipids in an amount from 0.0000001 to 2.0% b.w., more preferably in an amount from 0.0000001 to 1.0% b.w., more preferably from 0.0000001 to 0.5% b.w., based on the total weight of the extract.

In a preferred embodiment according to the invention, the extract comprises at least 20% b.w. of free fatty acids, based on the total weight of the extract. In a further preferred embodiment according to the invention, the extract comprises at least 25% b.w. of free fatty acids, based on the total weight of the extract.

Preferably, the Gardner color value of the extract is below 7, more preferably below 5 at an extract concentration of 0.018% in oil. The Gardner Color Value or Gardner Color Scale is a one-dimensional scale used to measure the shade of the color yellow.

Preferably, the ash content of the extract is below 0.4%, more preferably below 0.3%, based on the total weight of the extract.

Colors of transparent liquids have been studied visually since the early 19th century. Changes in color can indicate contamination or impurities in the raw materials, process variations, or degradation of products over time. The yellowness of the transparent liquid is determined by pouring the sample into a tube and comparing it to a pre-determined and known standard. The standard that the sample falls closest to then becomes the value for the liquid. Gardner Color can also be measured by a dual beam xenon flash spectrophotometer, for example the ColorQuest XT or Hach Lange Lico 690 instrument. Spectrophotometers measure the percent transmittance of the product and automatically calculate and provide the Gardner color number using illuminant C and 2° observer.

The CIELAB color space (also known as CIE L*a*b* or sometimes called “Lab”) is a color space defined by the International Commission on Illumination (abbreviated CIE) in 1976. It expresses color as three values: L* for perceptual lightness, and a* and b* for the four unique colors of human vision: red, green, blue, and yellow. In a preferred embodiment according to the invention, the extracts have a L* value from 83 to 101. In a further preferred embodiment according to the invention, the extracts have a a* value from −8.8 to −0.7. In a further preferred embodiment according to the invention, the extracts have a b* value from 0.1 to 14.5.

In a further preferred embodiment according to the invention, the extract comprises not more than 27% b.w. of long chain alkenones, based on the total weight of the extract. Further preferred, the extract comprises not more than 22% b.w. of long chain alkenones, based on the total weight of the extract. By removing the alkenones from the extracts, content of free fatty acids and sterols are increased, as these compounds are well soluble in ethanol. In a further preferred embodiment according to the invention. the extract comprises not more than 15% b.w. of long chain alkenones, based on the total weight of the extract.

Further preferred are extracts according to the invention that comprise at least 0.25% b.w. of sterols, more preferably at least 0.29% b.w. of sterols, more preferably at least 0.35% b.w. of sterols, each weight percentage value based on the total weight of the extract. Further preferred are extracts that comprise at least 0.25% b.w. of ergosterol, more preferably at least 0.29% b.w. of ergosterol, more preferably at least 0.35% b.w. of ergosterol, each weight percentage value based on the total weight of the extract.

The extract can be used as such. The extract can also be used as liquid mixture by adding at least one cosmetically acceptable solvent, such as e.g. plant oils, triglycerides, mineral oil, fatty alcohols, fatty acid esters, silicone oil, ethanol and mixtures of two or more of these solvents to the extract and optionally removing unsoluble or precipitating extract constituents by a suitable process such as filtration, separation or centrifugation. Such liquid mixtures are often much easier to handle than the extract as such and are readily further processable in particular for cosmetic purposes. These liquid mixtures can optionally be prepared with the addition of a solubilizing agent, preservative, stabilizer or antioxidant.

Optional benefit of preparing such a liquid mixture is the reduction of less well soluble extract constituents and thereby improve use and formulation properties.

The extract can also be used as solid mixture formed by adding at least one cosmetically acceptable solid carrier and optionally a solvent to the extract and then optionally drying the mixture by suitable processes such as e.g. spray-drying or vacuum drying. In this context, such a solid which is at least not toxic to the organisms on which it is to be used is cosmetically acceptable. Preferred solids are hydrocolloids such as starches, degraded or chemically or physically modified starches (in particular dextrins and maltodextrin), lactose, modified celluloses, gum arabic, gum ghatti, tragacanth gum, karaya, carrageenan, pullulan, curdlan, xanthan gum, gellan gum, guar gum, locust bean gum, alginates, agar, pectin, inulin or glucose and mixtures of two or more of these solids.

The extract can also be mixed with medium polar to lipophilic removable organic solvent such as e.g. a C1 to C4 alcohol, most preferably ethanol, n-propanol or iso-propanol, ethyl actetate or acetone, optionally under heating to 50-80° C. followed by cooling, remove the non-dissolved or re-precipitated solids, optionally adding a liquid or solid cosmetically acceptable carrier and optionally remove the removable organic solvent by e.g. distillation to obtain either a solvent- and carrier-free extract or an extract containing liquid or solid mixture.

The extract or the liquid or solid mixture comprising the extract can optionally also be further processed by encapsulation with a solid shell material, which is preferably chosen from starches, degraded or chemically or physically modified starches (in particular dextrins and maltodextrins), gelatines, wax materials, liposomes, gum arabic, agar-agar, ghatti gum, gellan gum, modified and non-modified celluloses, pullulan, curdlan, carrageenans, algic acid, alginates, pectin, inulin, xanthan gum and mixtures of two or more of the substances mentioned.

The solid shell material is preferably selected from gelatins (preferably including at least one gelatin having a Bloom value of greater than or equal to 200, preferably having a Bloom value of greater than or equal to 240, maltodextrin (preferably obtained from maize, wheat, tapioca or potato, preferred maltodextrins displaying a DE value in the range from 10 to 20), modified cellulose (e.g. cellulose ether), alginates (e.g. Na alginate), carrageenan (beta-, iota-, lambda- and/or kappa-carrageenan), gum arabic, curdlan and/or agar-agar. Gelatin is used in particular because of its good availability in various Bloom values. Production can take place as described for example in EP 0 389 700 A, JP 7 196 478, U.S. Pat. Nos. 4,251,195, 6,214,376, WO 03/055587 or WO 2004/050069.

The liquid, solid or encapsulated mixture obtainable or obtained according to the present invention comprise 0.0001 to 20 wt. %, preferably 0.001 to 10 wt % and most preferably 0.05-5 wt % SFE extract relative to the total mixture.

Method

A further embodiment according to the invention relates to a method for obtaining the supercritical fluid CO2 extract described above, comprising the steps of extracting cell material of Isochrysis sp. and/or Tisochrysis sp., preferably Tahitian Isochrysis with supercritical CO2, wherein the extraction comprises or consists of
a) exposition of the cell material to the supercritical CO2 for up to 24 h, preferably for up to 8 h at a temperature of 30 to 70° C. and a pressure of 150 to 290 bar.

Thus, in the context of the present invention the “cell material of Isochrysis sp.” and particularly “cell material of Tahitian Isochrysis” refers to a composition of thermal dried, spray dried, vacuum dried or freeze-dried, substantially or completely intact cells or mixtures thereof, wherein the cells are Isochrysis sp. cells or Tahitian Isochrysis cells, respectively. The cell material can comprise a carrier medium, provided that the total content of disrupted cells is less than 10% of all cells, preferably as determined as propidium iodide staining. In summary, what is extracted according to the present invention is not a homogenized or substantially disrupted mass of cells. Instead, the cell material according to the present invention is preferably obtained by a method comprising or consisting of the following steps:
a) Cultivating Isochrysis sp. cells and/or preferably of Tahitian Isochrysis cells,
b) Harvesting the cells to obtain completely or substantially intact cell material,
c) Optionally washing the cell material of step b) once or multiple times, to obtain washed, substantially or completely intact cell material,
d) Optionally freeze-drying the cell material of step b and/or step c, or
e) Optionally spray-drying the cell material of step b and/or c), or
f) Optionally thermal drying the cell material of step b and/or c), or
g) Optionally vacuum drying the cell material of step b and/or c),
h) Optionally milling the cell material of step b) and/or step c and/or step d) and/or step e) and/or step f) and/or step g).

Isochrysis sp., preferably Tahitian Isochrysis, is employed for obtaining extracts and compositions according to the present invention. Tahitian Isochrysis is a strain of Isochrysis collectable at Mataiva (Tahiti). According to the present invention, strain CS 177 obtainable from the Australian CSIRO collection (also registered as CCMP1324 at Provasoli-Guillard National Center for Culture of Marine; NEPCC601 at the Canadian Center for the Culture of Microorganisms) is preferably used. This strain has been isolated by K. Haines in 1977 at Mataiva, Tahiti.

According to the present invention, cell material of Isochrysis sp., preferably Tahitian Isochrysis, is extracted with supercritical CO2. These extractant has provided best results for increasing pigmentation of human skin and/or hair.

For extraction, the cell material is in step a) contacted with supercritical CO2 for up to 24 h at a temperature of 30 to 70° C. and a pressure of 150 to 290 bar. Also, exposition of the cell material to the extractant preferably lasts for up to 24 h, more preferably for 1-10 h and most preferably for 2-8 h.

After extraction, in step b) a pasty solid is obtained as an extract. The extract can be used as a composition for increasing pigmentation of human skin and/or hair, or, more preferably, is further processed into such composition as detailed below. The extracts collected can also be used for another exposition to supercritical CO2 in step a). The extraction thus preferably comprises repeating steps a) and b) once, twice, three or four times.

To measure the color of the extracts, the extracts can be dissolved at 0.018 wt.-% in vegetable oil triglyceride (INCI name: Caprylic/Capric Triglyceride), optionally in the presence of 0.01% tocopherol. As described above, the color can be measured by using the L*a*b* system as well as the Gardner value (Hach Lange Lico 690 instrument).

The present invention also relates to a method for increasing the pigmentation of human skin and/or hair (meaning scalp hair, eye lashes, eye brows, beard or other hair), comprising or consisting of the following steps:
a) providing a composition consisting of or comprising the supercritical fluid CO2 extract of Isochrysis sp. and/or Tisochrysis sp, preferably Tahitian Isochrysis as described above,
b) applying the composition to the skin and/or hair.

As further illustrated in the Example section, it can be seen that the extracts according to the invention clearly increase the mean cutaneous pigmentation score and thus increase the pigmentation of human skin and/or pigmentation of human hair. Furthermore, as compared with extracts according to the state of the art, the extracts according to the invention are both less colored and even more active in increasing the mean cutaneous pigmentation score.

The present invention further relates to a method for increasing skin hydration comprising or consisting of the following steps:
a) providing a composition consisting of or comprising the supercritical fluid CO2 extract of Isochrysis sp. and/or Tisochrysis sp, preferably Tahitian Isochrysis as described above,
b) applying the composition to the skin and/or hair.

Furthermore, the present invention relates to a method for reducing oxidative stress induced changes of skin and/or hair, comprising or consisting of the following steps:
a) providing a composition consisting of or comprising the supercritical fluid CO2 extract of Isochrysis sp. and/or Tisochrysis sp, preferably Tahitian Isochrysis as described above,
b) applying the composition consisting of or comprising the to the skin and/or hair.

In a preferred embodiment according to the invention, the oxidative stress is Maillard reaction- or Maillard reaction product-induced, particular dihydroxyacetone and/or erythrulose-induced oxidative stress and radicals. Therefore, in a preferred embodiment the present invention also relates to a method for reducing Maillard reaction- or Maillard reaction product-induced, in particular dihydroxyacetone and/or erythrulose-induced, radicals and oxidative stress and associated changes of skin and/or hair, comprising or consisting of the following steps:
a) providing a composition consisting of or comprising the supercritical fluid CO2 extract of Isochrysis sp. and/or Tisochrysis sp, preferably Tahitian Isochrysis as described above,
b) applying the composition to the skin and/or hair prior to and/or in combination with at least one Maillard reaction accessible reducing sugar, in particular dihydroxyacetone and/or erythrulose.
c) optionally adding to the composition prior to applying it to the skin and/or hair at least one UV filter.

A “reducing sugar” is any sugar that is capable of acting as a reducing agent. “A Maillard reaction accessible reducing sugar” is any sugar that is capable of acting as a reducing agent in Maillard reaction.

As already stated above, this is especially advantageous as a combination of fast acting self-tanners acting via Maillard reaction like dihydroxyacetone and/or erythrulose and the slower acting biological tanning extract of the invention provide a consumer expected visual tan in faster time than the extract of the invention alone. Preferably, the amount of the extract is 0.000002 to 2 wt. %, more preferred 0.00001 to 0.4 wt. %, even more preferred 0.00002 to 0.2 wt. %, most preferred 0.0002 to 0.1% based on the total weight of the composition.

In a preferred embodiment according to the invention, the at least one Maillard reaction accessible reducing sugar is added in an amount of from 0.1 to 20 wt. %, more preferably from 0.5 to 15 wt. %, even more preferably from 1 to 10 wt. %, most preferred 1 to 5% based on the total weight of the composition. Preferably, the at least one Maillard reaction accessible reducing sugar is dihydroxyacetone and/or erythrulose. Preferably, the ratio between the extract and the at least one Maillard reaction accessible reducing sugar is 1:1 000 000 to 1:5, more preferably 1:100 000 to 1:50, even more preferred 1:10 000 to 1:75, most preferably 1:5 000 to 1:100.

In a further preferred embodiment according to the invention, the oxidative stress is UV-induced oxidative stress. Thus, in a preferred embodiment the present invention relates to a method for reducing UV-induced radicals and oxidative stress and associated changes of skin and/or hair, comprising or consisting of the following steps:
a) providing a composition consisting of or comprising the supercritical fluid CO2 extract of Isochrysis sp. and/or Tisochrysis sp, preferably Tahitian Isochrysis as described above,
b) applying the composition to the skin and/or hair prior to and/or during UV exposure optionally in combination with at least one UV filter.

In another embodiment, the present invention also relates to a method for improving the cutaneous barrier properties comprising or consisting of the following steps:
a) providing a composition consisting of or comprising the supercritical fluid CO2 extract of Isochrysis sp. and/or Tisochrysis sp, preferably Tahitian Isochrysis as described above,
b) applying the composition to the skin and/or hair.

In another embodiment according to the invention, the present invention relates to a method for influencing or modifying human skin or human hair, wherein influencing or modifying human skin or human hair is selected from the group consisting of increasing pigmentation of human skin and/or pigmentation of human hair and/or reducing oxidative stress induced changes when applied to the skin and/or increasing skin hydration, and/or improving the cutaneous barrier properties of the skin, comprising or consisting of the following steps:
a) providing a composition consisting of or comprising the supercritical fluid CO2 extract of Isochrysis sp. and/or Tisochrysis sp, preferably Tahitian Isochrysis as described above,
b) applying the composition to the skin and/or hair,
c) optionally in combination with at least one UV filter and/or at least one tanner.

In a preferred embodiment according to the invention, the oxidative stress is UV-induced oxidative stress. In a further preferred embodiment according to the invention, the oxidative stress is Maillard reaction induced oxidative stress.

Preferably, the tanner is selected from acetyl tyrosine, D-chiro-inositol, dihydroxy methylchromonyl palmitate, pea extract, dihydroxyacetone and/or erythrulose. More preferred, the tanner is selected from acetyl tyrosine, dihydroxyacetone and/or erythrulose.

As can be seen in the example section, the extracts according to the invention relevantly upregulate aquaporin 3 (AQP3), catalase (CAT) and Involucrin (IVL) and also have an upregulating effect on hyaluronan synthase 2 (=HAS2).

Aquaporins (AQPs) are a family of homologous water transporting proteins expressed in many mammalian epithelial, endothelial and other cell types. AQP3 is the most abundant aquaporin in the epidermis and was shown to play an important role in skin hydration as water- and glycerol-transporting channel. AQP3 has also been found to be important in the function of the epidermal water permeability barrier [W. B. Bollag et al., Am. J. Physiol cell Physio1.2020, https://doi.org/10.1152/a jpce11.00075.2020]. Induction of AQP3 expression can therefore be expected to improve cutaneous hydration.

Oxidative stress in skin plays a major role in the aging process. The sources of reactive oxygen species (ROS), enzymatic as well as non-enzymatic, in skin cells are manifold. As the skin is at the interface between the exterior and the interior, external factors also contribute to ROS production in the skin. To cope with these many sources of ROS the skin has developed sophisticated and in part very skin-specific anti-oxidative mechanisms. Most of the anti-oxidants show in fact a higher concentration in the epidermis than in the dermis which correlates well with the fact that the ROS load is higher in the epidermis than in the dermis. Catalase is one of the enzymes that can handle reactive oxygen species and detoxifies hydrogen peroxide to produce water and oxygen. Catalasae is very prominently expressed in the skin, especially in the stratum corneum [M. Rinnerthaler et al., Biomolecules 2015, 5(2), 545-589]. T-ISO SFE CO2 extract is thus a radical scavenger, additionally upregulates antioxidant enzyme catalase and can therefore be expected to improve the oxidative stress level and reduce ROS-induced damages.

CD44 is the most well-studied hyaluronic acid (HA) receptor and the predominant receptor for HA on the cell surface of keratinocytes. Matrix HA is the major glycosaminoglycan in the extracellular matrix (ECM) of most mammalian tissues, including epidermis and dermis, and HA has been implicated in several skin epidermal functions. Down-regulation of CD44 in cultured keratinocytes (using CD44 siRNA) also significantly inhibits HA mediated keratinocyte differentiation and lipid synthesis [L. Y. Bourguignon et al., J. Invest. Dermatol. 2006, 1356-1365]. CD44 generally upregulates pro-proliferative and migratory effects of cells in tissues that contain abundant HA. HA levels and/or the interactions of HA and CD44 are able to regulate cellular differentiation (e.g., the cornification of epidermal keratinocytes and the differentiation of fibroblasts into myofibroblasts). During normal tissue homeostasis, hyaluronan synthesis and degradation in the epidermis are active, but balanced. However, whenever this homeostasis is disturbed with insults such as wounding, barrier disruption, or UVB radiation, epidermal hyaluronan content is rapidly increased. An increased expression of CD44 which is seen after epidermal insults closely correlates with hyaluronan accumulation. HA acting together with its receptor CD44 supports cell survival and stimulated HA synthesis through upregulated HA synthase expression is an inherent feature of the keratinocyte activation triggered by tissue trauma, and presumably important for a proper healing response. CD44 also appears to have a role in limiting inflammatory responses, which has also been shown in inflammation models.

Involucrin (IVL) is expressed in differentiating keratinocytes and incorporated into the cornified envelope. Experimental work in human skin has shown that IVL expression is increased following barrier disruption by acetone in a pattern to that seen in psoriatic skin and atopic skin, suggesting that IVL plays an important role cutaneous barrier properties and is involved in barrier repair [E. A. Brettmann et al., Exp Dermatol. 2018, 27(8), 859-866].

HAS2 is one of the three characterized hyaluronic acid (HA) synthases responsible for the polymerization of HA in the extracellular matrix and is the only essential gene of the family. Although all three isozymes are equally capable of synthesizing HA polymers, it has been shown that HAS2 is the main HA synthase polymerizing long hyaluronan chains of MW ˜2×106 Daltons. HA is the key molecule involved in skin hydration due to its unique capacity in retaining water.

Taken together the results of examples 7, 8, 9, 13 and 14, T-150 SFE CO2 extract can be expected to improve skin's and/or hair's well-being by reducing oxidative stress induced changes, increasing skin and/or hair hydration and improving cutaneous barrier properties.

Use

The present invention further relates to the use of a composition consisting of or comprising the supercritical fluid CO2 extract of Isochrysis sp. and/or Tisochrysis sp described above for increasing pigmentation of human skin and/or hair (meaning scalp hair, eye lashes, eye brows, beard or other hair).

Furthermore, the present invention relates to the use of a composition consisting of or comprising the supercritical fluid CO2 extract of Isochrysis sp. and/or Tisochrysis sp described above for increasing skin hydration.

In another embodiment, the present invention also relates to the use of a composition consisting of or comprising the supercritical fluid CO2 extract of Isochrysis sp. and/or

Tisochrysis sp described above for reducing oxidative stress induced changes when applied to the skin.

In another embodiment, the present invention also relates to the use of a composition consisting of or comprising the supercritical fluid CO2 extract of Isochrysis sp. and/or Tisochrysis sp described above for reducing Maillard reaction- or Maillard reaction product-induced oxidative stress and associated changes when applied to the skin. This is especially advantageous as a combination of fast acting self-tanners acting via Maillard reaction like dihydroxyacetone and/or erythrulose and the slower acting biological tanning extract of the invention provide a consumer expected visual tan in faster time than the extract of the invention alone.

Preferably, the amount of the extract is 0.000002 to 2 wt. %, more preferred 0.00001 to 0.4 wt. %, even more preferred 0.00002 to 0.2 wt. %, most preferred 0.0002 to 0.1 wt. % based on the total weight of the composition.

Preferably, the Maillard reaction- or Maillard reaction product-induced oxidative stress and associated changes are induced by at least one Maillard reaction accessible reducing sugar. This sugar can be applied to the skin prior to and/or in combination to the composition. In a preferred embodiment according to the invention, the at least one Maillard reaction accessible reducing sugar is added in an amount of from 0.1 to 20 wt. %, more preferably from 0.5 to 15 wt. %, even more preferably from 1 to 10 wt. %, most preferably from 1 to 5 wt. % based on the total weight of the composition. Preferably, the at least one Maillard reaction accessible reducing sugar is dihydroxyacetone and/or erythrulose. Preferably, the ratio between the extract and the at least one Maillard reaction accessible reducing sugar is 1:1 000 000 to 1:5, more preferably 1:100 000 to 1:50, even more preferably 1:10 000 to 1:75, most preferably 1:5 000 to 1:100.

In another embodiment, the present invention also relates to the use of a composition consisting of or comprising the supercritical fluid CO2 extract of Isochrysis sp. and/or Tisochrysis sp described above for reducing UV-induced oxidative stress and associated changes when applied to the skin.

Moreover, the present invention relates to the use of a composition consisting of or comprising the supercritical fluid CO2 extract of Isochrysis sp. and/or Tisochrysis sp described above for improving the cutaneous barrier properties.

In one embodiment, the invention relates to the use of a composition consisting of or comprising the supercritical fluid CO2 extract of Isochrysis sp. and/or Tisochrysis sp as described above for influencing or modifying human skin and/or human hair, wherein the extract is used for increasing pigmentation of human skin and/or pigmentation of human hair and/or for reducing oxidative stress induced changes when applied to the skin and/or for increasing skin hydration, and/or for improving the cutaneous barrier properties of the skin.

In a preferred embodiment according to the invention, the oxidative stress is UV-induced oxidative stress. In a further preferred embodiment according to the invention, the oxidative stress is Maillard reaction induced oxidative stress.

Preferably, the Maillard reaction induced oxidative stress is oxidative stress induced by dihydroxyacetone and/or erythrulose.

As described above, the extracts according to the invention relevantly upregulate aquaporin 3 (AQP3), catalase (CAT) and Involucrin (IVL) and also have an upregulating effect on hyaluronan synthase 2 (=HAS2).

In a preferred embodiment according to the invention, the supercritical fluid CO2 extract is an extract of Tahitian Isochrysis.

It is clearly stated that the uses described above only relate to cosmetic uses.

Cosmetic Composition

Compositions according to the present invention can advantageously be combined, in particular in cosmetic products, with further conventional components, such as, for example: preservatives, in particular those described in US 2006/0089413, antimicrobial agents, such as e.g. antibacterial agents or agents to treat yeast and mold, in particular those described in WO 2005/123101, compounds against ageing of the skin, in particular those described in WO 2005/123101, antidandruff agents, in particular those described in WO 2008/046795, antioxidants, in particular those described in WO2005/123101, carrier materials, in particular those described in WO 2005/123101, chelating agents, in particular those described in WO 2005/123101, deodorizing agents and antiperspirants, in particular those described in WO 2005/123101, moisture regulators (moisture-donating agents, moisturizing substance, moisture-retaining substances), in particular those described in WO 2005/123101, osmolytes, in particular those described in WO 2005/123101, compatible solutes, in particular those described in WO 01/76572 and WO 02/15868, proteins and protein hydrolysates, in particular those described in WO 2005/123101 and WO2008046676, skin-tanning agents, in particular those described in WO 2006/045760, cooling agents, in particular those described in WO 2005/123101, skin-cooling agents, in particular those described in WO 2005/123101, warming agents, in particular those described in WO 2005/123101, UV-absorbing agents, in particular those described in WO 2005/123101, UV filters, in particular those described in WO 2005/123101, benzylidenebeta-dicarbonyl compounds in accordance with WO 2005/107692 and alpha-benzoyl-cinnamic acid nitriles in accordance with WO 2006/015954, insect repellents, in particular those described in WO 2005/123101, plant parts, plant extracts, in particular those described in WO 2005/123101, vitamins, in particular those described in WO 2005/123101, emulsifiers, in particular those described in WO 2005/123101, gelling agents, in particular those described in WO 2005/123101, oils in particular those described in WO 2005/123101, waxes in particular those described in WO 2005/123101, fats in particular those described in WO 2005/123101, phospholipids, in particular those described in WO 2005/123101, saturated fatty acids and mono- or polyunsaturated fatty acids and α-hydroxy-acids and polyhydroxy-fatty acids and esters of saturated and/or unsaturated branched and/or unbranched alkane carboxylic acids, in particular those described in WO 2005/123101, surface-active substances (surfactants) in particular those described in WO 2005/123101, skin repair agents comprising cholesterol and/or fatty acids and/or ceramides and/or pseudoceramides, in particular those described in WO 2006/053912, dyestuffs and colorants and pigments, in particular those described in WO 2005/123101, aroma chemicals and flavors and fragrances, in particular those described in S. Arctander, Perfume and Flavor Chemicals, private publishing house, Montclair, N.J., 1969 and Surburg, Panten, Common Fragrance and Flavor Materials, 5th Edition, Wiley-VCH, Weinheim 2006, preferably those explicitly mentioned in US 2008/0070825, alcohols and polyols, in particular those described in WO 2005/123101, organic solvents, in particular those described in WO 2005/123101, silicones and silicone oils and silicone derivatives in particular those described in WO2008046676, virucides, abrasives, anti-cellulite agents, astringents, antiseptic agents, antistatics, binders, buffers, cell stimulants, cleansing agents, care agents, depilatory agents, softeners, enzymes, essential oils, in particular those described in US 2008/0070825, fibres, film-forming agents, fixatives, foam-forming agents, foam stabilizers, substances for preventing foaming, foam boosters, gelforming agents, hair growth activators, hair growth inhibitors, hair care agents, hair-setting agents, hair-straightening agents, hair-smoothening, bleaching agents, strengthening agents, stain-removing agents, optically brightening agents, impregnating agents, dirt-repellent agents, friction-reducing agents, lubricants, opacifying agents, plasticizing agents, covering agents, polish, gloss agents, polymers in particular those described in WO2008046676, powders, peptides, mono-, di- and oligosaccharides, re-oiling agents, abrading agents, skin-soothing agents, skincleansing agents, skin care agents, skin-healing agents, skin-protecting agents, skin-softening agents, skin-smoothing agents, nourishing agents, skin-warming agents, stabilizers, detergents, fabric conditioning agents, suspending agents, thickeners, yeast extracts, algae or microalgae extracts, animal extracts, liquefiers, color-protecting agents, anticorrosives and electrolytes.

In one embodiment, the invention relates to a cosmetic composition comprising the supercritical fluid CO2 extract of Isochrysis sp. and/or Tisochrysis sp, preferably of Tahitian Isochrysis as described above and at least one further ingredient selected from the group consisting of:
a) tanner
b) skin moisturizing agents,
c) stabilizer, and/or
d) antioxidants
e) UV filters.

Advantageous skin and hair tanning active ingredients in this respect are artificial tanners or browning agents such as dihydroxyacetone or other reducing sugars, erythrulose, carotenoids, extracts of fresh green walnut shells or henna, but also biological tannining agents such as substrates or substrate analogues of tyrosinase such as L-tyrosine, N-acetyl tyrosine, L-DOPA or L-dihydroxyphenylalanine, xanthine alkaloids such as caffeine, theobromine and theophyl-line and derivatives thereof, proopiomelanocortin peptides such as ACTH, alpha-MSH, peptide analogues thereof and other substances which bind to the melanocortin receptor, MelinOlL (a liposoluble form of an α-MSH biomimetic peptide, INCI: Isopropyl Palmitate, Lecithin, Water, Acetyl Hexapeptide-1), Dihydroxy Methylchromonyl Palmitate, peptides such as Val-Gly-Val-Ala-Pro-Gly, Lys-Ile-Gly-Arg-Lys or Leu-Ile-Gly-Lys, purines, pyrimidines, folic acid, copper salts such as copper gluconate, chloride or pyrrolidonate, 1,3,4-oxadiazole-2-thiols such as 5-pyrazin-2-yl-1,3,4-oxadiazole-2-thiol, curcumin, zinc diglycinate (Zn(Gly)2), manganese(II) bicarbonate complexes (“pseudocatalases”) as described for example in EP 0 584 178, tetrasubstituted cyclohexene derivatives as described for example in WO 2005/032501, isoprenoids as described in WO 2005/102252 and in WO 2006/010661, melanin derivatives such as Melasyn-100 and MelanZe, diacyl glycerols, aliphatic or cyclic diols, psoralens, prostaglandins and analogues thereof, activators of adenylate cyclase and compounds which activate the transfer of melanosomes to keratinocytes such as serine proteases or agonists of the PAR-2 receptor, extracts of plants and plant parts of the chrysanthemum species, sanguisorba species, walnut extracts, pea extract, Monk's pepper (Vitex agnus-castus), urucum extracts, rhubarb extracts, microalgae extracts, and trehalose. Flavonoids which bring about skin and hair tinting or browning (e.g. quercetin, rhamnetin, kaempferol, tiliroside, fisetin, genistein, daidzein, chrysin and apigenin, epicatechin, diosmin and diosmetin, morin, quercitrin, naringenin, hesperidin, phloridzin and phloretin) can also be used.

Most preferred tanning or browning agents are dihydroxyacetone, erythrulose, acetyl tyrosine, dihydroxy methylchromonyl palmitate and/or pea extract. Even more preferred are acetyl tyrosine, dihydroxyacetone and erythrulose. Most preferred are dihydroxyacetone and erythrulose.

The amount of the aforementioned examples of additional active ingredients for the modulation of skin and hair pigmentation (one or more compounds) in the products according to the invention is then preferably 0.00001 to 30 wt. %, preferably 0.0001 to 20 wt. %, particularly preferably 0.001 to 8 wt. %, based on the total weight.

Preferably, the amount of the extract is 0.000002 to 2 wt. %, more preferred 0.00001 to 0.4 wt. %, even more preferred 0.00002 to 0.2 wt. %, most preferred 0.0002 to 0.1 wt. %, based on the total weight of the composition.

Preferably, the amount of the tanner is from 0.00001 to 20 wt. %, preferably 0.0001 to 15 wt. %, particularly preferably 0.001 to 10 wt. % and most preferred 0.05 to 5 wt. %, based on the total weight of the composition. When dihydroxyacetone and/or erythrulose is in a preferred embodiment used as a tanner, the amount is from 0.1 to 20 wt. %, more preferably from 0.5 to 15 wt. %, even more preferably from 1 to 10 wt. %, most preferably from 1 to 5 wt. %, based on the total weight of the composition. Preferably, the tanner is dihydroxyacetone and/or erythrulose. In case, the cosmetic composition comprises the supercritical fluid CO2 extract of Isochrysis sp. and/or Tisochrysis sp, preferably of Tahitian Isochrysis as described above and dihydroxyacetone and/or erythrulose as tanner, the ratio between the extract and the tanner is 1:1 000 000 to 1:5, more preferably 1:100 000 to 1:50, even more preferably 1:10 000 to 1:75, most preferably 1:5 000 to 1:100.

Preferred skin moisturizing agents are selected from the group consisting of alkane diols or alkane triols comprising 3 to 12 carbon atoms, preferably C₃-C₁₀-alkane diols and C₃-C₁₀-alkane triols. More preferably the skin moisturizing agents are selected from the group consisting of: glycerol, 1,2-propylene glycol, 1,2-butylene glycol, 1,3-butylene glycol, 1,2-pentanediol, 1,2-hexanediol, 1,2-octanediol and 1,2-decanediol.

The moisturizing agents can be added to the composition in an amount of preferably 0.00001 to 30 wt. %, preferably 0.0001 to 20 wt. %, particularly preferably 0.001 to 10 wt. %, based on the total weight of the composition.

Preferred stabilizers are selected from the group consisting of chelating agents, preferably EDTA, disodium EDTA, tetrasodium EDTA, trisodium MGDA (methylglycinediacetic acid), tetrasodium glutamate diacetate, trisodium ethylenedia mine disuccinate, ubiquinone and ubiquinol and their derivatives, phytic acid and α hydroxy acids (for example citric acid, lactic acid, malic acid), quenchers, preferably tris (tetramethylhydroxypiperidinol)citrate (trade name: Tinogard QS) and photostabilzers, preferably Benzotriazolyl Dodecyl p-cresol (trade name: Tinogard TL), Sodium Benzotriazolyl Butylphenol Sulfonate (trade name: Tinogard HS), Diethylhexyl Syringylidene Malonate (trade name: Oxynex ST), Bumetrizole (trade name: Tinogard AS), Benzophenone-4 (trade name: Uvinul MS 40) and Benzotriazolyl Butylphenol Sulfonate (trade name: Cibafast H liquid).

The stabilizers can be added to the composition in an amount of preferably 0.00001 to 30 wt. %, preferably 0.0001 to 20 wt. %, particularly preferably 0.001 to 5 wt. %, based on the total weight of the composition.

Antioxidants such as for example tocopherol (vitamin E) or tocopherol mixtures, tocopherol acetate, tocopheryl succinate, tocopheryl linoleate, vitamin A and its derivatives (for example Vitamin A palmitate, Hydroxypinacolone Retinoate) tert-butylhydroquinone (TBHQ), butylhydroxytoluol (BHT), butylhydroxyanisole, hydroxymethoxyphenyl decanone, 6-paradol (Hydroxymethoxyphenyl Decanone), imidazoles (for example urocanic acid) and their derivatives, cannabidiol and its extracts (Cannabis sativa seed oil, Cannabis sativa extract), peptides such as D,L-carnosine, D-carnosine, L-carnosine and their derivatives (for example anserine), hydroxyphenyl propamidobenzoic acid (dihydro avenanthramide D), diethylhexyl syringylidene malonate, phenylethyl resorcinol, gallic acid and their derivatives, hydroxyacetophenone, rosmarinic acid, lipoic acid and its derivatives (for example dihydrolipoic acid), or other suitable antioxidants are also easily incorporated.

Most preferred are tocopherol (vitamin E) or tocopherol mixtures, tocopherol acetate, tocopheryl succinate, tocopheryl linoleate, vitamin A and its derivatives (for example Vitamin A palmitate, Hydroxypinacolone Retinoate) tert-butyl hydroquinone (TBHQ), butylhydroxytoluol (BHT), 6-paradol (Hydroxymethoxyphenyl Decanone), cannabidiol, hydroxyacetophenone, caronsine and diethylhexyl syringylidene malonate.

The antioxidants can be added to the composition in an amount of preferably 0.00001 to 30 wt. %, preferably 0.0001 to 20 wt. %, particularly preferably 0.001 to 5 wt. %, based on the total weight of the composition.

In some embodiments according to the invention the composition further comprises UV filters. Suitable UV filters are, for example, organic UV absorbers from the class of 4-aminobenzoic acid and derivatives, salicylic acid derivatives, benzophenone derivatives, dibenzoylmethane derivatives, diphenylacrylates, 3-imidazol-4-ylacrylic acid and its esters, benzofuran derivatives, benzylidenemalonate derivatives, polymeric UV absorbers containing one or more organosilicon radicals, cinnamic acid derivatives, camphor derivatives, trianilino-s-triazine derivatives, 2-hydroxyphenylbenzotriazole derivatives, menthyl anthranilate, benzotriazole derivatives and indole derivatives.

Specific UV filters which can be used are for example as follows:
UVB filters:
p-aminobenzoic acid
ethyl p-aminobenzoate (25 mol) ethoxylated
2-ethylhexyl p-dimethylaminobenzoate
homomenthyl salicylate (homosalate) (Neo Heliopan® HMS)
2-ethylhexyl salicylate (octisalate) (Neo Heliopan® OS)
triethanolamine salicylate (Neo Heliopan® TS)
menthyl anthranilate (Neo Heliopan® MA)
2-ethylhexyl p-methoxycinnamate (Neo Heliopan® AV)
isoamyl p-methoxycinnamate (Neo Heliopan® E 1000)
2-phenylbenzimidazole sulfonic acid (Neo Heliopan® Hydro) and its salts
3-(4′-trimethylammonium)benzylidenebornan-2-one methyl sulphate
3-(4′-sulpho)benzylidenebornan-2-one and salts
3-(4′-methylbenzylidene)-d,l-camphor (Neo Heliopan® MBC)
N-[(2 and 4)-[2-(oxoborn-3-ylidene)methyl]benzyl]acrylamide polymer
4,4′-[(6-[4-(1,1-dimethyl)aminocarbonyl)phenylamino]-1,3,5-triazine-2,4-diyl)diimino]
bis(benzoic acid 2-ethylhexyl ester) (Uvasorb® HEB)
benzylidenemalonate-polysiloxane (Parsol® SLX)
tris(2-ethylhexyl)4,4′,4″-(1,3,5-triazine-2,4,6-triyltriimino)tribenzoate (Uvinul® T150)
2-ethylhexyl 2-cyano-3,3-diphenylacrylate (Neo Heliopan®303)

Broadband Filters Such as, for Example:
2-hydroxy-4-methoxybenzophenone-5-sulfonic acid (sulisobenzone, benzophenone-4) or its salts.
2-hydroxy-4-methoxybenzophenone (Neo Heliopan® BB, Oxybenzone, benzophenone-3 disodium 2,2′-dihydroxy-4,4′-dimethoxy-5,5′-disulphobenzophenone phenol,-(2H-benzotriazol-2-yl-4-methyl-6-(2-methyl-3-(1,3,3,3-tetramethyl-1-(trimethylsilyl)oxy)disiloxyanyl)propyl), (Mexoryl® XL)
2,2′-methylenebis(6-(2H-benztriazol-2-yl)-4-1,1,3,3-tetramethylbutyl)-phenol), Tinosorb® M)
2,4-bis[4-(2-ethylhexyloxy)-2-hydroxyphenyl]-1,3,5-triazine
2,4-bis[[(4-(2-ethylhexyloxy)-2-hydroxy]phenyl]-6-(4-methoxyphenyl)-1,3,5-triazine, (Neo Heliopan® BMT)
Tris-Biphenyl Triazine (Tinosorb® A2B)
2,4-bis[[(4-(3-sulphonato)-2-hydroxypropyloxy)-2-hydroxy]phenyl]-6-(4-methoxyph enyl)-1,3,5-triazine sodium salt
2,4-bis[[(3-(2-propyloxy)-2-hydroxypropyloxy)-2-hydroxy]phenyl]-6-(4-methoxyphenyl)-1,3,5-triazine
2,4-bis[[4-(2-ethylhexyloxy)-2-hydroxy]phenyl]-6-[4-(2-methoxyethyl carbonyl)phenylamino]-1,3,5-triazine
2,4-bis[[4-(3-(2-propyloxy)-2-hydroxypropyloxy)-2-hydroxy]phenyl]-6-[4-(2-ethylcarboxyl)phenylamino]-1,3,5-triazine
2,4-bis[[4-(2-ethylhexyloxy)-2-hydroxy]phenyl]-6-(1-methylpyrrol-2-yl)-1,3,5-triazine
2,4-bis[[4-tris(trimethylsiloxysilylpropyloxy)-2-hydroxy]phenyl]-6-(4-methoxyphenyl)-1,3,5-triazine
2,4-bis[[4-(2″-methylpropenyloxy)-2-hydroxy]phenyl]-6-(4-methoxyphenyl)-1,3,5-triazine
2,4-bis[[4-(1′,1′,1′,3′,5′,5′,5′-heptamethylsiloxy-2″-methylpropyloxy)-2 hydroxy]phenyl]-6-(4-methoxyphenyl)-1,3,5-triazine.
(5,6,5′,6′-teraphenyl-3,3′-(1,4-Phenylene)bis(1,2,4-triazine),

UVA Filters are for Example the Following:
terephthalylidenedibornanesulphonic acid and salts (Mexoryl® SX)
Avobenzone (Neo Heliopan® 357)
hexyl 2-(4-diethylamino-2-hydroxybenzoyl)benzoate (Uvinul® A Plus)
menthyl anthranilate (Neo Heliopan® MA)

In a preferred embodiment according to the invention the composition further comprises at least one UV filter selected from the group consisting of Butyl Methoxydibenzoylmethane, Ethylhexyl Salicylate, Ethylhexyl Methoxycinnamate, Isoamyl p-Methoxycinnamate, Homosalate, Octocrylene, Phenylbenzimidazole sulfonic acid, Bis-ethylhexyloxyphenol methoxyphenyl triazine or Benzophenone-3. In a more preferred embodiment according to the invention the composition further comprises at least one UV filter selected from the group consisting of Butyl Methoxydibenzoylmethane, Ethylhexyl Salicylate, Homosalate, Octocrylene, Bis-Ethylhexyloxyphenol Methoxyphenyl Triazine, Ethylhexyl Methoxycinnamate or Isoamyl p-Methoxycinnamate. In a most preferred embodiment according to the invention the composition further comprises at least one UV filter selected from the group consisting of Butyl Methoxydibenzoylmethane, Ethylhexyl Salicylate, Homosalate, Bis-Ethylhexyloxyphenol Methoxyphenyl Triazine or Octocrylene.

It is possible, furthermore, to use particulate UV filters or inorganic pigments, which if desired may have been rendered hydrophobic, such as the oxides of zinc (ZnO), of oxides of titanium (TiO2) of iron (Fe2O3), of zirconium (ZrO2), of silicon (SiO2), of manganese (e.g. MnO), of aluminium (Al2O3), of cerium (e.g. Ce2O3) and/or mixtures. In a further preferred embodiment according to the invention the composition further comprises one or more of Zinc Oxide, Titanium Dioxide, Octinoxate or Ensulizole.

In one embodiment according to the invention the amount of UV-filters is in the range of 0.1 to 55% by weight, preferably in the range from 0.3 to 45% by weight, more preferably in the range from 0.5 to 35% by weight, and most preferably in the range from 1.0 to 31% by weight, based on the total weight of the composition.

In one embodiment according to the invention the total amount of oil soluble UV filters that can be used, which are, for example but not limited to Butyl Methoxydibenzoylmethane, and/or Bis-Ethylhexyloxyphenol Methoxyphenyl Triazine, and/or Isoamyl p-Methoxycinnamate, and/or 2-ethylhexyl salicylate, and/or homosalate, and/or Ethylhexyl Methoxycinnamate, and/or octocrylene, is in the range of 0.1 to 55% by weight, particularly in the range of 0.3 to 45% by weight, more particularly in the range of 0.5 to 35% by weight, most particularly in the range of 1 to 31% by weight, based on the total weight of the composition.

Preferably, the ratio between the extract of the invention and the amount of UV filters is 1:2 000 000 to 1:20, more preferably 1:200 000 to 1:50, even more preferably 1:50 000 to 1:75, most preferably 1:10 000 to 1:100.

In a preferred embodiment according to the invention, the cosmetic composition comprises the supercritical fluid CO2 extract of Isochrysis sp. and/or Tisochrysis sp, preferably of Tahitian Isochrysis as described above and at least two further ingredients selected from the group consisting of:
a) tanner
b) skin moisturizing agents,
c) stabilizer, and/or
d) antioxidants
e) UV filters.

In a further preferred embodiment according to the invention, the cosmetic composition comprises the supercritical fluid CO2 extract of Isochrysis sp. and/or Tisochrysis sp, preferably of Tahitian Isochrysis as described above and at least three further ingredients selected from the group consisting of:
a) tanner
b) skin moisturizing agents,
c) stabilizer, and/or
d) antioxidants
e) UV filters.

In a further preferred embodiment according to the invention, the cosmetic composition comprises the supercritical fluid CO2 extract of Isochrysis sp. and/or Tisochrysis sp, preferably of Tahitian Isochrysis as described above and additional ingredients selected from the group consisting of:
a) 0.001 to 20 wt. % tanner
b) 0.001 to 10 wt. % skin moisturizing agents,
c) 0.001 to 5 wt. % stabilizer, and/or
d) 0.001 to 5 wt. % antioxidants
e) 0.1 to 55 wt. % UV filters,
all weight percentages based on the total weight of the cosmetic composition.

It goes without saying that all of the preferred embodiments mentioned above also apply here. The invention is further described by the following figures and examples, without limiting the scope of the claims.

### EXAMPLES

### Example 1: Extraction of Freeze-Dried, Milled Tahitian Isochrysis (T-ISO) by Supercritical Fluid Extraction with Carbondioxide

Freeze-dried T-ISO biomass (cultivated in photobioreactors under natural light in Italy, characterized by a loss on drying as determined by use of an infrared/halogen dryer of 4.2% and supplied by Archimede Ricerche s.r.l., Italy), was pre-treated using a mechanical three roller mill and then extracted with supercritical CO2 in a supercritical fluid extraction unit equipped with a 1 L extraction vessel at different conditions:
A) 223 g biomass, extraction for 1 h at 150 bar and 60° C. followed by extraction for another 2 hours at 290 bar and 60° C.
B) 223 g biomass, extraction for 2 h at 150 bar and 60° C.

The extracts were collected in a separator and the weight loss of biomass was used to determine the extraction yield.

TABLE 1

| | | Extraction yield | Total yield |
|---|---|---|---|
| Set-up | Conditions | [wt.-%] | [wt.-%] |
| A-1 | 1 h, 150 bar, 60° C. | 7.1 | 14.6 |
| A-2 | 2 h, 290 bar, 60° C. | 7.5 | |
| B-1 | 2 h, 150 bar, 60° C. | 10.4 | 10.4 |

The supercritical fluid extracts were analytically characterized:
Free fatty acids by HPLC (detection at 210 nm or by ELSD) including myristic (C14:0), palmitic (C16:0), oleic (C18:1), linolic (C18:2), linolenic (C18:3), stearidonic (C18:4) and docosahexaenoic (C22:6) acid
Sum of glycerol bound fatty acids by fatty acid methyl esters (FAMEs) analysis consisting of saponification of the lipids to produce salts of the free fatty acids, derivatization of the free acids to form methyl esters and analysis of the obtained methyl esters by gas chromatography (GC)
Sum of C37/38 alkenones by GC using C34 alkane as internal standard
Sum of sterols by GC using C34 alkane as internal standard including ergosterol and stigmasterol
total carotenoids and total chlorophylls by spectrophotometric determination considering readings at wavelengths of 750 and 480 nm and at wavelengths of 750, 663, 645 and 630 nm, respectively (T. T. Parsons, J. D. H. Strickland, J. Mar. Res. 1963, 21, 155-163)

The results are summarized in table 2.

TABLE 2
Results of the analytical characterization
of the supercritical fluid extracts

| Extract/ | Content [wt.-%] | | |
|---|---|---|---|
| Substance class | A-1 | A-2 | B-1 |
| Free fatty acids | 32.8 | 15.4 | 35.2 |
| Glycerol bound fatty acids | 10.5 | 7.2 | 13.5 |
| C37/38 Alkenones | 21.9 | 15.3 | 26.8 |
| Sterols | 0.37 | 0.46 | 0.49 |
| Total carotenoids | 0.01 | 0.86 | 0.07 |
| Total chlorophylls | 0.01 | 0.98 | 0.03 |

To compare the color of the extracts, they were dissolved at 0.018 wt.-% in vegetable oil triglyceride (INCI name: Caprylic/Capric Triglyceride) in the presence of 0.01% tocopherol.

Color was measured by using the L*a*b* system as well as the Gardner value (Hach Lange Lico 690 instrument).

TABLE 3
Comparison of color of the extracts

| Extract | L* value | a* value | b* value | Gardner value |
|---|---|---|---|---|
| A-1 | 100.1 | −0.7 | 2.7 | 0.2 |
| A-2 | 99.8 | −1.8 | 7.7 | 0.9 |
| B-1 | 96.2 | −8.1 | 45.3 | 5.6 |

The results show that all 3 extractions lead to extracts with low color. When measured at 0.018% in oil all 3 lead to Gardner values <9 and L* values >83.

Modulation of Skin Pigmentation by SFE Extracts A-1 and A-2:

Organ culture of human skin was performed starting from a skin sample, exciding pieces of approximately 8×3 mm (ø×thickness) and culturing them up to day 6. Skin samples (6 per treatment) were cultured in an air-liquid interface in a perforated ring of stainless steel in contact with culture medium (modified Williams' E medium). The culture medium was renewed at the day 3.

Samples of the super critical fluid (SFE) extracts were dissolved in ethanol. Both the extract samples and the control (ethanol) were applied topically and renewed daily. For application the skin biopsies were gently cleaned with a cotton pad and then 4 μl of each test sample or control were applied on top of each piece and covered with a 6 ø mm delivery membrane.

After 6 days of organ culture, histological section were prepared from the skin samples and the quantitative changes of melanin content were investigated following Fontana-Masson staining technique. The melanin quantification was obtained by image analysis of microphotographs of each histological skin section.

Table 4 summarizes the mean pigmentation score

TABLE 4
Mean pigmentation score

| | Mean | Modulation of |
|---|---|---|
| | pigmentation | pigmentation vs |
| Test sample | score | control |
| Control (ethanol) | 85.6 | — |
| 0.2 ppm A-1 | 89.4 | +4% |
| 2.0 ppm A-1 | 103.4 | +21% |
| 0.2 ppm A-2 | 99.2 | +16% |
| 2.0 ppm A-2 | 99.4 | +16% |

The results clearly show that SFE extracts A-1 and A-2 both increased the mean cutaneous pigmentation score (at 2.0 ppm+21 and +16%, respectively). Even very small amounts (0.2 ppm) of both extracts lead to an increase of the mean cutaneous pigmentation score.

### Example 2: Extraction of Freeze-Dried, Milled Tahitian Isochrysis (T-ISO) by Supercritical Fluid Extraction with Carbondioxide and Comparison to Extraction by Ethyl Acteate/Reproduction of Extraction as Described in EP2168570

As the pigmentation increasing activity was present in both SFE extracts of example 1, extraction was performed under constant conditions of pressure and temperature. For this, 8 kg of freeze-dried T-ISO biomass (cultivated in photobioreactors under natural light in Italy, characterized by a loss on drying as determined by use of an infrared/halogen dryer of 5.4% and supplied by Archimede Ricerche s.r.l., Italy) were pre-treated using a mechanical three roller mill and then extracted with supercritical CO2 in a supercritical fluid extraction unit equipped with a 45 L extraction vessel at 250 bar and 48° C. and at a constant CO2 flow rate of 200 kg/hour for 6-8 hours. A green-brown pasty solid was obtained and extraction yield was 10%.

For direct comparison, an ethyl acetate was prepared according to the description given in EP2168570 in small scale using 11 g of the same T-ISO biomass. After removal of the ethyl acetate from the filtered, clear extract solution, a dark green, viscous solid was obtained with an extraction yield of 17%.

Extracts were analytically characterized as given in example 1 and results are summarized in table 5.

TABLE 5
Comparison of CO2 and Ethyl acetate extract

Content [wt.-%]

| Extract/ | | Ethyl acetate |
|---|---|---|
| Substance class | CO2 extract | extract |
| Free fatty acids | 42.3 | 26.1 |
| Glycerol bound fatty acids | 7.5 | 7.2 |
| C37/38 Alkenones | 14.7 | 14.8 |
| Sterols | 0.56 | 0.93 |
| Including | | |
| Ergosterol | 0.53 | 0.85 |
| Stigmasterol | 0.03 | 0.08 |
| Total | 0.12 | 0.74 |
| carotenoids | | |
| Total | 0.11 | 3.94 |
| chlorophylls | | |

The results show that the SFE CO2 extract contains a lower content of total carotenoids and chlorophylls and a higher content of free fatty acids compared to the ethyl acetate extract.

To compare the color of the extracts, they were dissolved at 0.018 wt.-% in vegetable oil triglyceride (INCI name: Caprylic/Capric Triglyceride) in the presence of 0.01% tocopherol.

Color was measured by using the L*a*b* system as well as the Gardner value (Hach Lange Lico 690 instrument). The results are showed in table 6.

TABLE 6
Comparison of color of CO2 and Ethyl acetate extract

| Extract | L* value | a* value | b* value | Gardner value |
|---|---|---|---|---|
| CO2 extract | 98.8 | −3.3 | 14.3 | 2.2 |
| Ethyl acetate | 81.1 | −7.3 | 125.2 | 11.4 |
| extract | | | | |

The results show that the color, most significantly the b* and Gardner value, of the CO2 extract is much lower compared to the ethyl acetate extract.

To compare the modulatory activity of skin pigmentation by both extracts, organ culture of human skin was performed as described in example 1. Results are summarized in table 7.

TABLE 7
Mean pigmentation score of CO2 and Ethyl acetate extract

| | Mean | Modulation of |
|---|---|---|
| | pigmentation | pigmentation vs |
| Test sample | score | control |
| Control (ethanol) | 126.2 | — |
| 0.2 ppm CO2 extract | 157.9 | +25% |
| 2.0 ppm CO2 extract | 162.0 | +28% |
| 0.2 ppm ethyl acetate | 141.2 | +12% |
| extract | | |
| 2.0 ppm ethyl acetate | 151.1 | +20% |
| extract | | |

The results clearly show that the ethyl acetate extract as described in EP2168570 increased the mean cutaneous pigmentation score. However surprisingly, the less colored CO2 extract was even more active than the more intense colored ethyl acetate extract (at 0.2 ppm+25% versus+12% and at 2.0 ppm+28 versus+20%, respectively).

### Example 3: Extraction of Freeze-Dried, Non-Milled Tahitian Isochrysis (T-ISO) by Supercritical Fluid Extraction with Carbondioxide

1.5 kg of freeze-dried T-ISO biomass (same batch as used in example 2), was extracted without milling as pre-treatment with supercritical CO2 in a supercritical fluid extraction unit equipped with a 3 L extraction vessel at constant conditions of pressure (200 bar) and temperature (48° C. A brown-green pasty solid was obtained and extraction yield was 13%.

For comparison, the extraction of another 1.5 kg of freeze-dried T-ISO biomass was performed at 250 bar and 48. A clearly more intensively colored green-brown pasty solid was obtained and extraction yield was 15%.

Extracts were analytically characterized as given in example 1. Additionally, the ash content of the extracts was determined. Results are summarized in table 8.

TABLE 8
Results of the analytical characterization
of the supercritical fluid extracts

Content [wt.-%]

| Extract/ | CO2 extract, | CO2 extract, |
|---|---|---|
| Substance class | 200 bar | 250 bar |
| Free fatty acids | 41.9 | 44.8 |
| Glycerol bound fatty acids | 7.0 | 5.6 |
| C37/38 Alkenones | 16.8 | 16.0 |
| Sterols | 0.68 | 0.81 |
| Including | | |
| Ergosterol | 0.64 | 0.76 |
| Stigmasterol | 0.03 | 0.05 |
| Total carotenoids | 0.05 | 0.26 |
| Total chlorophylls | 0.15 | 0.48 |
| Ash | 0.27 | 0.00 |

Isochrysis galbana extract was described by D. L. Alonso et al. (Phytochem. 1998, 47, 1473-1481) to contain apart from neutral lipids also glycolipids and phospholipids with phosphatidylcholines being the most abundant. Therefore, the extracts were analyzed in parallel to L-alpha-phosphatidylcholine (Sigma, CAS 8022-43-5) by HPLC-MS/MS in positive ion mode and the MS2 ion chromatogram for 184 corresponding to the characteristic fragment of this substance class was extracted.

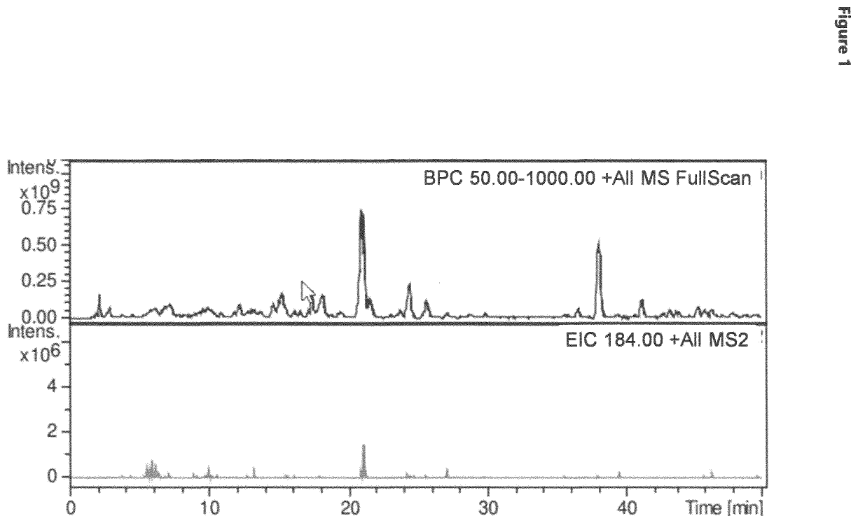

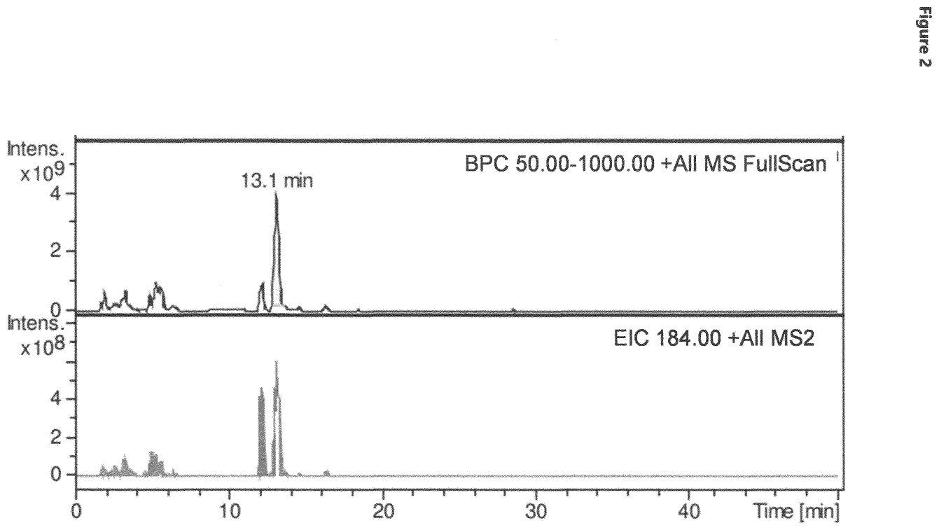

HPLC-MS analysis of the T-ISO SFE CO2 extract 200 bar (FIG. 1) and L-alpha-phosphatidylcholine (FIG. 2) dissolved in tetrahydrofuran (THF)/ethanol 1+4 (v+v) was performed using the following method:
Column+pre-column: Agilent Eclipse XDB-C8, S-5 μm, 150×4.6 mm+10×4.6 mm
Temperature: 40° C.
Flow: 0.8 ml/min
Solvent A: methanol/water/formic acid (750/250/1)
Solvent B: acetonitrile/methanol/isopropanol/formic acid (400/300/300/1)
Gradient:

| time | % A | % B |
|---|---|---|
| 0 | 100 | 0 |
| 45 | 30 | 70 |
| 50 | 22 | 78 |

Injection volume: 5 μl
Ammonium formate was added before entry into the ESI ion trap by syringe pump to facilitate ionization.

FIG. 1 shows ESI+base peak chromatogram (top) and ESI+extracted ion chromatogram (EIC) MS2 184 (bottom) of T-ISO SFE CO2 extract, 200 bar, 1.13% in THE/ethanol 1+4 (v/v).

FIG. 2 shows ESI+base peak chromatogram (top) and ESI+extracted ion chromatogram (EIC) MS2 184 (bottom) of L-alpha-phosphatidylcholine, 0.50 in THE/ethanol 1+4 (v/v).

L-alpha-phosphatidylcholine (FIG. 2) showed as expected a peak at 12.0 and 13.1 min in both the ESI+base peak chromatogram and the ESI+extracted ion chromatogram MS2 184. At a concentration of 0.5%, the peak in the MS2 EIC reached an intensity of about 5×10⁸ units.

The T-ISO SFE CO2 extract, 200 bar at a concentration of 1.13% showed intensive peaks in the range of 10⁹ to 10⁸ units in the ESI+base peak chromatogram. However, only very small peaks reaching an intensity of only about 0.5×10⁶ units were observed in the expected region between 10 and 20 min indicating that phosphatidylcholines if at all are only present in trace amounts.

This finding is also expected, as supercritical fluid extraction with CO2 alone is known to extract phospholipids in general only in traces or not at all.

Furthermore, the T-ISO SFE extract, 200 bar was investigated by ¹³C NMR analysis for the presence of glycolipids. Glycolipids are characterized by a sugar residue in the molecule which would be detectable by a characteristic signal of the anomeric C atom in the region of 100 to 110 ppm in the ¹³C NMR spectrum.

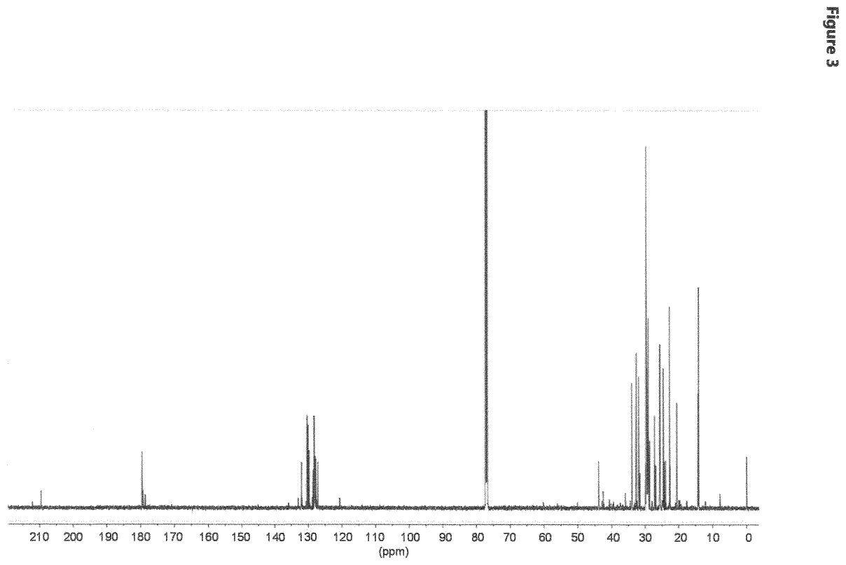

FIG. 3 shows ¹³C NMR spectrum (600 MHz) of T-ISO SFE extract, 200 bar in CDCl₃.

The ¹³C NMR spectrum (FIG. 3) shows no signal in the expected region of 100 and 110 ppm indicating that glycolipids if at all are only present in trace amounts.

This finding is also expected, as supercritical fluid extraction with CO2 alone is known to extract glycolipids in general only in traces or not at all.

To compare the color of the extracts, they were dissolved at 0.018 wt.-% in vegetable oil triglyceride (INCI name: Caprylic/Capric Triglyceride) in the presence of 0.01% tocopherol.

Color was measured by using the L*a*b* system as well as the Gardner value (Hach Lange Lico 690 instrument).

TABLE 9
Comparison of color of the extracts

| Extract | L* value | a* value | b* value | Gardner value |
|---|---|---|---|---|
| CO2 extract, 200 | 99.2 | −3.0 | 12.0 | 1.7 |
| bar | | | | |
| CO2 extract, 250 | 94.8 | −7.4 | 47.6 | 5.8 |
| bar | | | | |

Organ culture of human skin was performed as described in example 1 and the ethyl acetate extract of example 2 was tested in parallel for direct comparison on the same skin sample.

Results are summarized in table 10.

TABLE 10
Comparison of mean pigmentation score

| | Mean | |
|---|---|---|
| | pigmentation | Modulation |
| Test sample | score | vs control |
| Control (ethanol) | 150.3 | — |
| 0.2 ppm CO2 extract, | 166.1 | +10% |
| 200 bar | | |
| 2.0 ppm CO2 extract, | 171.9 | +14% |
| 200 bar | | |
| 0.2 ppm CO2 extract, | 186.5 | +24% |
| 250 bar | | |
| 2.0 ppm CO2 extract, | 184.4 | +23% |
| 250 bar | | |
| 0.2 ppm ethyl acetate | 163.0 | +8% |
| extract of example 2 | | |
| 2.0 ppm ethyl acetate | 162.4 | +8% |
| extract of example 2 | | |

The results clearly show that both SF CO2 extracts increase the mean pigmentation score and are both more active than the significantly more colored ethyl acetate prepared from the same raw material batch. The SF CO2 extract obtained at 250 bar is more active but also more colored than the SF CO2 extract obtained at 200 bar.

### Example 4: Extraction of Spray-Dried, Non-Milled Tahitian Isochrysis (T-ISO) by Supercritical Fluid Extraction with Carbondioxide and Comparison to Ethyl Acetate Extract

1.3 kg of spray-dried T-ISO biomass (cultivated in photobioreactors under natural light in Italy, characterized by a loss on drying as determined by use of an infrared/halogen dryer of 4.3% and supplied by Archimede Ricerche s.r.l., Italy), was extracted without milling as pre-treatment with supercritical CO2 in a supercritical fluid extraction unit equipped with a 3 L extraction vessel at constant conditions of pressure (200 bar) and temperature (48° C.). A green-brown pasty solid was obtained and extraction yield was 11%.

For direct comparison, an ethyl acetate was prepared according to the description given in EP2168570 in small scale using 26 g the same T-ISO biomass. After removal of the ethyl acetate from the filtered, clear extract solution, a dark green, viscous solid was obtained with an extraction yield of 15%.

Extracts were analytically characterized as given in example 1. Additionally the ash content of the T-ISO SFE extract was analyzed. Results are summarized in table 11.

TABLE 11
Comparison of CO2 and Ethyl acetate extract

| | |
|---|---|
| Extract/ | Content [wt.- %] |

| Substance class | CO2 extract | Ethyl acetate exract |
|---|---|---|
| Free fatty acids | 43.5 | 25.2 |
| Glycerol bound fatty acids | 6.7 | 6.9 |
| C37/38 Alkenones | 19.0 | 12.5 |
| Sterols | 1.26 | 2.20 |
| Including | | |
| Ergosterol | 0.58 | 0.58 |
| Stigmasterol | 0.75 | 0.81 |
| Total carotenoids | 0.10 | 1.11 |
| Total chlorophylls | 0.15 | 5.94 |
| Ash | 0.10 | Not analyzed |

To compare the color of the extracts, they were dissolved at 0.018 wt.-% in vegetable oil triglyceride (INCI name: Caprylic/Capric Triglyceride) in the presence of 0.01% tocopherol.

Color was measured by using the L*a*b* system as well as the Gardner value (Hach Lange Lico 690 instrument).

TABLE 12
Comparison of color

| Extract | L* value | a* value | b* value | Gardner value |
|---|---|---|---|---|
| CO2 extract | 99.2 | −3.8 | 14.3 | 2.1 |
| Ethyl acetate | 83.4 | −8.8 | 103.6 | 9.4 |
| extract | | | | |

The results clearly show that the extract solution of the CO2 extract is significantly lighter in color.

### Example 5: Liquid Mixture Comprising Tahitian Isochrysis Extract Obtained by Supercritical Fluid Extraction with Carbondioxide

Tahitian Isochrysis (T-ISO) supercritical fluid extract obtained with carbondioxide as described in example 2 was used to prepare an easy to handle and formulate liquid mixture in a cosmetically acceptable carrier. 2.0 g of the T-ISO SFE CO2 extract were added to 215 g of vegetable oil triglyceride (INCI name: Caprylic/Capric Triglyceride, density ˜0.95 kg/I) and dissolved under warming to 70-80° C. with stirring until the extract was completely dissolved. After let cooling to <40° C., 1.1 g of tocopherol was added under stirring as stabilizer. Afterwards the mixture was allowed to stand at a temperature of 15 to 25° C. for several days to allow re-precipitation of less well soluble extract ingredients. The formed precipitate was removed by filtration giving a clear liquid characterized by a water content of <0.1 wt-% and parameters as summarized in table 13.

TABLE 13
Characterization of extract

| | | | | | Relative | Refractive |
|---|---|---|---|---|---|---|
| Liquid mixture | | | | Gardner | density | index |
| (undiluted) | L* value | a* value | b* value | value | (D20/4) | (n20/D) |
| Directly after | 77.0 | 6.5 | 126.2 | 12.0 | 0.9459 | 1.4501 |
| preparation | | | | | | |
| After 20 weeks | 78.1 | 5.6 | 127.3 | 11.9 | Not analyzed | Not analyzed |
| storage at room | | | | | | |
| temperature | | | | | | |

Content of total carotenoids and total chlorophylls determined as described above was 0.015 mg/I and 0.013 mg/I, respectively.

Color measurement was repeated after storage for 20 weeks at room temperature and proved color stability as only minor differences versus start values were observed.

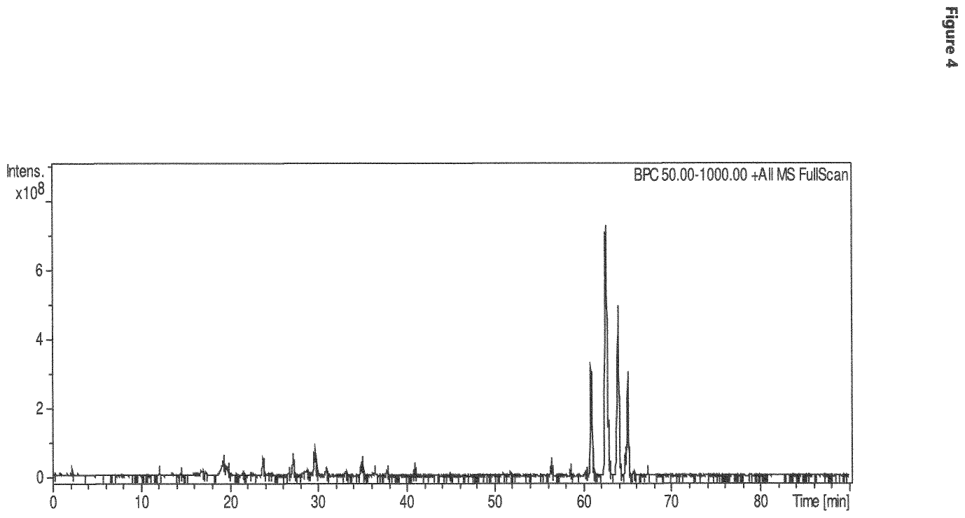

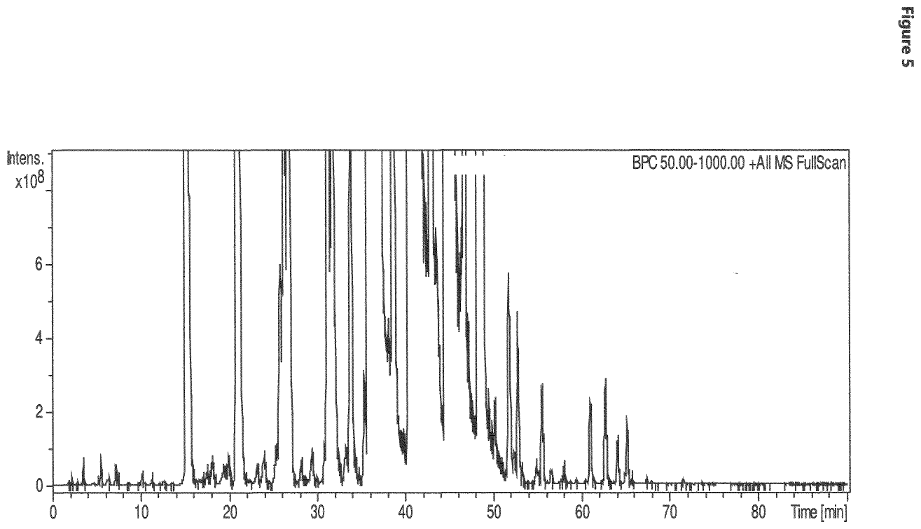

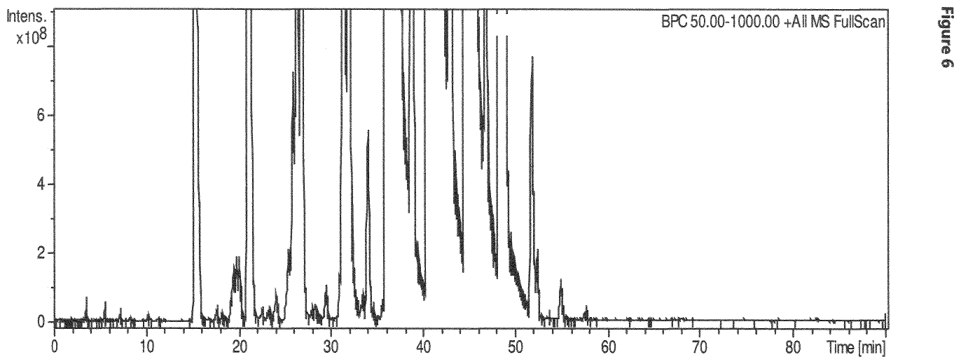

HPLC-MS analysis of the T-ISO SFE CO2 extract (FIG. 4), the liquid mixture after filtration (FIG. 5) and the vegetable oil triglyceride (FIG. 6) dissolved in tetrahydrofuran (THF)/ethanol 1+4 (v+v) was performed using the following method:
Column+pre-column: Agilent Eclipse XDB-C8, S-5 μm, 150×4.6 mm+10×4.6 mm
Temperature: 40° C.
Flow: 0.8 ml/min
Solvent A: methanol/water/formic acid (750/250/1)
Solvent B: acetonitrile/methanol/isopropanol/formic acid (400/300/300/1)
Gradient:

| time | % A | % B |
|---|---|---|
| 0 | 100 | 0 |
| 45 | 30 | 70 |
| 65 | 0 | 100 |
| 90 | 0 | 100 |

Injection volume: 5 μl
Ammonium formate was added before entry into the ESI ion trap by syringe pump to facilitate ionization.

FIG. 4 shows ESI+base peak chromatogram of T-ISO SFE CO2 extract, 0.09% in THF/ethanol 1+4 (v/v).

FIG. 5 shows ESI+base peak chromatogram of liquid mixture after filtration, diluted 1:10 with THF/ethanol 1+4 (v/v) corresponding to 0.09% T-ISO SFE CO2 extract (the same extract concentration as in A).

FIG. 6 shows ESI+base peak chromatogram of vegetable oil triglyceride, diluted 1:10 with THF/ethanol 1+4 (v/v).

Retention times (Rt) of long chain alkenones were between 60 and 66 min as shown in table 14.

TABLE 14
Retention times

| | m/z for | Molecular | | |
|---|---|---|---|---|
| Rt (min) | [M + NH4]+ | weight [g/mol] | Formula | Alkenone |
| 61.2 | 547 | 529 | C₃₇H₆₈O | C37:3Me |
| 62.9 | 549 | 531 | C₃₇H₇₀O | C37:2Me |
| 64.2 | 563 | 545 | C₃₈H₇₂O | C38:2Et |
| 65.1 | 577 | 559 | C₃₉H₇₄O | C39:2 |

C37:3Me = heptatriaconta-8,15,22-trien-2-one
C37:2Me = heptatriaconta-15,22-dien-2-one
C38:2Et = octatriaconta-16,25-dien-3-one

Comparison of the intensity of the long chain alkenones in the obtained chromatogram for the SFE extract (FIG. 4, 5 to 7×10⁸ units) versus the chromatogram obtained for the liquid mixture after filtration (FIG. 5, 1 to 3×10⁸ units) clearly showed that this compound class was significantly reduced in the liquid mixture.

The chromatogram of the vegetable oil triglyceride showed no peaks in the respective retention time range of 60 to 66 min.

The group of long chain alkenones is highly lipophilic and therefore very difficult to dissolve. Even if clearly dissolved in a specific solvent or solvent mixture, precipitation will occur after storage or after adding to typical cosmetic formulations, especially water comprising cosmetic formulations. Therefore, reduction of these alkenones in the T-ISO extract or in a mixture comprising T-ISO extract results in easier handling and improved use and formulation properties.

The same results were obtained when repeating the preparation of the liquid mixture using soybean oil (INCI name: Glycine Soja (Soybean) Oil, density ˜0.92 kg/I) instead of vegetable oil triglyceride.

Re-precipitation of less well soluble extract ingredients can be facilitated by cooling the liquid mixture to 5 to 0° C. or to −10 to −20° C. This shortens the time needed for this process step.

Re-precipitation of less well soluble extract ingredients can also be facilitated by increasing the dry extract content in the liquid mixture.

The re-precipitation facilitating conditions can optionally be used alone or also in combination.

Desirable dry extract contents of liquid mixtures are 0.5-5%, more desirable 0.5-3%.

Organ culture of human skin was performed as described in example 1. The liquid mixture in vegetable oil triglyceride and the SFE extract of example 2 as such was tested in parallel using ethanol as vehicle for direct comparison on the same skin sample. Results are summarized in table 15.

TABLE 15
Mean pigmentation after preparation and filtration

| | Mean | |
|---|---|---|
| | pigmentation | Modulation |
| Test sample | score | vs control |
| Control (ethanol) | 150.3 | — |
| 0.2 ppm extract from | 180.6 | +20% |
| example 2 | | |
| 2 ppm extract from | 174.8 | +16% |
| example 2 | | |
| 0.02% liquid mixture | 172.8 | +15% |
| (corresponding to | | |
| 0.18 ppm extract | | |
| of example 2) | | |
| 0.2% liquid mixture | 168.7 | +12% |
| (corresponding to | | |
| 1.8 ppm extract | | |
| of example 2) | | |

The results clearly show that the skin pigmentation increasing activity is maintained in the liquid mixture after preparation and filtration. The obtained liquid mixture is easy to handle and dose into typical cosmetic formulations. Addition of tocopherol provides stabilization of sensitive extract ingredients in the liquid mixture resulting in good storage stability. Removal of the highly lipophilic and therefore difficult to solubilize C37/38 alkenones prevents precipitations in typical water containing cosmetic formulations such as emulsion, creams, balms, lotions, gels and serums.

To evaluate the formulation properties of the obtained liquid mixture in vegetable oil triglyceride was formulated into an o/w emulsion and pH was adjusted to 5 and 7. Table 16 shows the formulation.

TABLE 16
Formulation example

| | | 5.1 | 5.2 | 5.3 | 5.4 |
|---|---|---|---|---|---|
| | INCI | w/w % | w/w % | w/w % | w/w % |
| Phase A | | | | | |
| Emulsiphos ® | Potassium Cetyl | 2.0 | 2.0 | 2.0 | 2.0 |
| | Phosphate, | | | | |
| | Hydrogenated Palm | | | | |
| | Glycerides | | | | |
| Cutina PES ex BASF | Pentaerythrityl Distearate | 1.5 | 1.5 | 1.5 | 1.5 |
| Cutina GMS ex BASF | Glyceryl Stearate | 2.0 | 2.0 | 2.0 | 2.0 |
| SymMollient ® S | Cetearyl Nonanoate | 3.0 | 3.0 | 3.0 | 3.0 |
| Vegetable oil | Caprylic/Capric/Triglceride | 7.0 | 7.0 | 7.0 | 7.0 |
| triglyceride | | | | | |
| Dragoxat 89 | Ethylhexyl Isononanoate | 3.0 | 3.0 | 3.0 | 3.0 |
| Phase B | | | | | |
| Ultrez-21 | Acrylates/C10-30 Alkyl | 0.2 | 0.2 | 0.2 | 0.2 |
| | Acrylate Crosspolymer | | | | |
| Keltrol CG | Xanthan Gum | 0.2 | 0.2 | 0.2 | 0.2 |
| Phase C | | | | | |
| Water, demin. | Water (Aqua) | Ad 100 | Ad 100 | Ad 100 | Ad 100 |
| EDTA BD | Disodium EDTA | 0.1 | 0.1 | 0.1 | 0.1 |
| Hydrolite ® 5 | Pentyleneglycol | 1.0 | 1.0 | 1.0 | 1.0 |
| SymDiol ® 68 | 1,2-Hexanediol, Caprylyl | 0.6 | 0.6 | 0.6 | 0.6 |
| | Glycol | | | | |
| Propylene Glycol | Propylene Glycol | 1.0 | 1.0 | 1.0 | 1.0 |
| Glycerine 86.5% | Glycerin | 1.0 | 1.0 | 1.0 | 1.0 |
| Phase D | | | | | |
| Sodium Hydroxide | Aqua, Sodium Hydroxide | 0.6 | 0.6 | 0.6 | 0.6 |
| 10% solution | | | | | |
| Phase E | | | | | |
| Liquid mixture | | 4 | 4 | 1 | 1 |
| Sum | | 100.0 | | | |
| pH value adjusted | | 5 | 7 | 5 | 7 |

Production method: Heat Phase A and C separately up to 80° C. Disperse Phase B in A. Add Phase C to AB and emulsify using an Ultra Turrax Stirrer. (3 min/6000 rpm) Add Phase D and neutralize. Allow to cool by using a vane stirrer.

Add liquid mixture to obtained O/W emulsion while stirring with a vane stirrer (100 rpm). Stir for 15 minutes. Adjust pH with sodium hydroxide (10% aqueous solution) to pH 7 and with citric acid (10% aqueous solution) to pH 5.

Colorimetric measurement of the formulations was performed at the beginning (t=0) and 6 months (x) after storage at room temperature (RT) or at 40° C. The difference of 2 colors AE can be calculated using the following equation:

ΔE*_{ab vs t=0}=\sqrt{(L*ₓ−L*ₜ₌₀)²+(a*ₓ−a*ₜ₌₀)²+(b*ₓ−b*ₜ₌₀)²}

A difference of ΔE of 0.5-1 can be visually observed by a trained evaluator by naked eye. A difference of 2-4 can be observed visually also by a non-trained evaluator.

Results are summarized in table 17:

TABLE 17
Results of colorimetric measurement

| | Formulation | | | | | | | |
|---|---|---|---|---|---|---|---|---|
| | 5.1 | | 5.2 | | 5.3 | | 5.4 | |
| | pH 5 | pH 7 | pH 5 | pH 7 | pH 5 | pH 7 | pH 5 | pH 7 |
| | Condition | | | | | | | |
| | RT | 40° C. | RT | 40° C. | RT | 40° C. | RT | 40° C. |
| L* value | | | | | | | | |
| t = 0 | 90.31 | 90.31 | 90.15 | 90.15 | 91.59 | 91.59 | 91.66 | 91.66 |
| t = 6 months | 89.08 | 89.53 | 88.72 | 89.57 | 91.11 | 91.05 | 90.32 | 90.58 |
| a* value | | | | | | | | |
| t = 0 | −3.34 | −3.34 | −3.38 | −3.38 | −2.16 | −2.16 | −2.16 | −2.16 |
| t = 6 months | −3.46 | −3.36 | −3.41 | −3.07 | −2.24 | −1.97 | −2.28 | −1.92 |
| b* value | | | | | | | | |
| t = 0 | 14.76 | 14.76 | 15.34 | 15.34 | 6.19 | 6.19 | 6.18 | 6.18 |
| t = 6 months | 15.12 | 12.93 | 14.71 | 13.10 | 6.37 | 4.76 | 6.40 | 5.06 |
| ΔE | 1.29 | 1.99 | 1.56 | 2.33 | 0.52 | 1.54 | 1.36 | 1.57 |

Color results as well as color differences ΔE show that the color of the o/w emulsions containing 4 and 1% of the liquid mixture with vegetable oil triglyceride and adjusted to either pH 5 or pH 7 are stable for 6 months storage at room temperature or at 40° C.

Furthermore, all o/w emulsions were analyzed under the microscope directly after preparation and after 6 months of storage at the different conditions for occurrence of crystals. No crystals or crystallization was observed at any time point and at any storage condition proving again the good formulation and storage properties of the obtained liquid mixture.

### Example 6: Ex Vivo Skin Pigmentation Increasing Efficacy of a Cosmetic Formulation Containing Liquid Mixtures of T-ISO Extracts

For evaluation of the ex vivo skin pigmentation increasing efficacy of a T-ISO SFE extract containing cosmetic formulation, the liquid mixture described in example 5 with composition given in table 18 was used:

TABLE 18
Composition

| | Liquid mixture |
|---|---|
| Ingredient | wt-% |
| T-ISO CO2 SFE extract | 0.9 |
| according to example 2 | |
| Tocopherol | 0.5 |
| vegetable oil triglyceride | Ad 100 |
| (INCI name: Caprylic/ | |
| Capric Triglyceride) | |

The following cosmetic formulations were prepared using this liquid mixture of table 19.

TABLE 19
Cosmetic formulations

| | | 6.1 | 6.2 |
|---|---|---|---|
| | INCI | w/w % | w/w % |
| Phase A | | | |
| PCL-Liquid 100 | Cetearyl Ethylhexanoate | 3.0 | 3.0 |
| Lanette O | Cetearyl Alcohol | 2.0 | 2.0 |
| Vegetable oil | Caprylic/Capric | 3.0 | 3.0 |
| triglyceride | Triglyceride | | |
| Eutanol G | Octyldodecanol | 4.0 | 4.0 |
| Abil 350 | Dimethicone | 0.5 | 0.5 |
| Phase B | | | |
| Pemulen TR-1 | Acrylates/C10-30 Alkyl | 0.2 | 0.2 |
| | Acrylate Crosspolymer | | |
| Carbopol Ultrez-21 | Acrylates/C10-30 Alkyl | 0.05 | 0.05 |
| Polymer | Acrylate Crosspolymer | | |
| Water | | Ad 100 | Ad 100 |
| Hydrolite ® 5 | Pentyleneglycol | 2.0 | 2.0 |
| Phase C | | | |
| Sodium Hydroxide 10% | Aqua, Sodium | 0.5 | 0.5 |
| solution | Hydroxide | | |
| Phase D | | | |
| Liquid mixture | | — | 1.0 |
| Sum | | 100.0 | 100.0 |

Production method: Heat phase B at 80° C. without Pemulen TR-1. Disperse Pemulen TR-1 by stirring. Heat phase A at 80° C. and add A to B with an Ultra Turrax. Allow to cool down at 40° C. Adjust the pH value with phase C at 6.

Add Phase D to obtain formulation 6.2.

The liquid mixture was easy to handle, well compatible with the formulation and no negative impact such as e.g. re-crystallization or modulation of odor was observed.

Organ culture of human skin was performed as described in example 1 to evaluate the skin pigmentation increasing efficacy of the 2 formulations versus untreated. Results are summarized in table 20.

TABLE 20
Comparison of mean pigmentation score untreated-
placebo-according to the invention

| | Mean | |
|---|---|---|
| | pigmentation | Modulation |
| Test sample | score | vs control |
| Untreated | 73.1 | — |
| Formulation 6.1 | 73.1 | 0% |
| (Placebo) | | |
| Formulation 6.2 (1.0% | 93.1 | +27% |
| liquid mixture) | | |

The results clearly show that the liquid mixture used in a cosmetic formulation at 1.0% stimulates skin pigmentation versus untreated as well as versus placebo.

Compared to organ culture of human skin (ex vivo), the effective concentration of the liquid mixture, and with this the effective concentration of the T-ISO SFE extract, is typically 2 to 5 fold higher when topically applied on in vivo human skin, i.e. 2 to 5 wt-% liquid mixture instead of the 1 wt-% in the above example. This is due to the different conditions in organ culture.

The impact of the 2 formulations on the skin viability was determinded by MTT [(3-(4,5-dimethylthiazol-2-yl)-2,5-diphenyltetrazolium bromide) tetrazolium reduction] assay performed at day 6 and compared to untreated. Neither the placebo (formulation 6.1) nor the formulation containing the liquid mixture (6.2) modulated skin viability versus untreated proving the good cutaneous tolerability of the T-ISO SFE extract and the liquid mixture containing it.

### Example 7: ABTS Assay

The radical scavenging, i.e. antioxidant capacity of the T-ISO SFE CO2 extract obtained at 200 bar and described in example 3 (test substance) was measured with the aid of the ABTS assay. 2,2′-azino-bis(3-ethylbenzothiazoline-6-sulphonic acid) (ABTS) was transformed by potassium persulphate into the blue-green radical cation ABTS·+. The radical cations were reduced by antioxidants (test substances), and discoloration took place which was determined photometrically at 734 nm.

$$Inhibition [ \% ] = 100 - ( \frac{A test substance}{A control} \times 100 )$$

where A test substance means absorption of the wells with the test substance and A control means absorption of the wells without the test substance.

The IC50 was calculated from the inhibition of radical formation [%] in a series of dilutions of the tested sample. This is the concentration at which radical formation is inhibited by 50%. The results are shown in Table 21.

TABLE 21
Results of raducal-scavenging activity

| T-ISO SFE CO2 extract, 200 bar | Radical-scavenging | |
|---|---|---|
| from example 3 | activity | |
| Test concentration [%] | [%] | IC50 [%] |
| 0.01 | 10 | 0.062 |
| 0.05 | 43 | |
| 0.1 | 68 | |

The results show that T-ISO SFE CO2 extract is a radical scavenger providing dose-dependent antioxidant activity.

### Example 8: Gene Expression of Keratinocytes

Neonatal human epidermal keratinocytes (nHEK) were cultivated in an EpiLife® medium (Gibco) including an HKGS kit (Gibco) with 5% CO2 at 37° C. in accordance with the supplier's instructions. The cells were treated for 24 hours, with 0.001% of T-ISO SFE CO2 extract of example 3 obtained at 200 bar dissolved in DMSO and DMSO alone as the vehicle control. Genomic target expression levels in treated cells were measured using a quantitative Real-Time PCR comparison to vehicle control treatment.

RNA was isolated using Qiagen's RNeasy® Mini Kit. The total RNA concentrations were measured using Eppendorf's μCuvetteG 1.0 and BioPhotometer, by measuring the absorption at 260 nm. Purity control values such as E260/280 and E260/230 were calculated simultaneously. Reverse transcription was performed using the high-capacity RNA-to-cDNA kit of Applied Biosystems, in accordance with the supplier's instructions. Samples were treated in Biometra's PCR Thermocycler. For fast real-time qPCR, the cDNA was diluted with RNase-free water, and the TaqMan™ Fast Universal PCR Master Mix of Applied Biosystems was used. Quantitative real time PCR was performed using the StepOnePlus fast real-time PCR instrument by Applied Biosystems. Analysis was conducted using the StepOne software and 2-ΔΔct method (normalised to endogenous control HTRP1 expression). For upregulations, RQ values≥2.0 are considered to be relevant.

TABLE 22
Results

| | RQ value |
|---|---|
| Gene | 0.001% Extract |
| HPRT1 (housekeeping) | 1.0 |
| AQP3 | 2.0 |
| CAT | 2.0 |
| CD44 | 2.1 |
| IVL | 4.1 |

The results show that the T-ISO SFE extract at 0.001% relevantly upregulates aquaporin 3 (AQP3), catalase (=CAT), CD44, and involucrin (=IVL).

### Example 9: Gene Expression of Fibroblasts

Normal human dermal fibroblasts (NHDF) were cultivated in DMEM medium and treated with 0.001% of the same extract for 24 hours. Afterwards fast real-time qPCR was performed as described in above using a customised gene array.

For modulations, RQ values ≥2.0 are considered to be relevant.

TABLE 23
Results

| | RQ value |
|---|---|
| Gene | 0.001% Extract |
| HPRT1 (housekeeping) | 1.0 |
| CAT | 3.2 |
| HAS2 | 1.8 |

The results show that the T-ISO SFE extract at 0.001% relevantly upregulates catalase (=CAT) and has a slight upregulating effect on hyaluronan synthase 2 (=HAS2, RQ value of 1.8).

### Example 10: T-ISO SFE Extract with Reduced Long Chain Alkenone Content

The T-ISO SFE extract prepared as described in example 3 at 200 bar was weight into a flask and ethanol was added to obtain extract solutions as given in Table 24. The mixture was stirred under heating to 50-600. Afterwards it was allowed to cool to ambient temperature (18 to 22° C.) overnight. The formed precipitate was separated by filtration and the clear filtrate was analyzed for fingerprint pattern by HPLC using the method as described above but with charge aerosol detection instead of ESI+. For the analysis, an aliquot of the filtrates (max 100 μl) were taken and analyzed at the same extract concentration.

The remaining filtrate was evaporated to obtain the dried filtrate and quantitative determinations as given in table 24 were performed.

TABLE 24

| | T-ISO SFE | | | | |
|---|---|---|---|---|---|
| | extract | 1 | 2 | 3 | 4 |
| SFE extract | — | 328 mg | 514 mg | 567 mg | 679 mg |
| amount used | | | | | |
| Ethanol | — | 46.5 g | 25.7 g | 11.3 g | 6.8 g |
| amount used | | | | | |
| Wt-% | — | 0.9 | 2 | 5 | 10 |
| obtained | | | | | |
| solution | | | | | |
| Amount dried | — | 246 mg | 377 mg | 388 mg | 421 mg |
| supernatant | | | | | |
| after | | | | | |
| separation | | | | | |
| Yield dried | — | 75% | 73% | 68% | 62% |
| supernatant | | | | | |
| Free fatty | 30.4 | 36.0 | 37.0 | 37.6 | 38.6 |
| acids | | | | | |
| Glycerol | n.a | n.a. | n.a. | n.a. | n.a. |
| bound fatty | | | | | |
| acids | | | | | |
| C37/38 | 11.6 | 1.7 | 1.2 | 0.7 | 0.5 |
| Alkenones | | | | | |
| Sterols | 0.29 | 0.36 | 0.39 | 0.39 | 0.40 |
| Including | 0.28 | 0.35 | 0.37 | 0.37 | 0.39 |
| Ergosterol | | | | | |
| Total | 0.03 | 0.01 | 0.01 | 0.01 | 0.02 |
| carotenoids | | | | | |
| Total | 0.15 | 0.11 | 0.12 | 0.12 | 0.14 |
| chlorophylls | | | | | |

n.a. = not analyzed

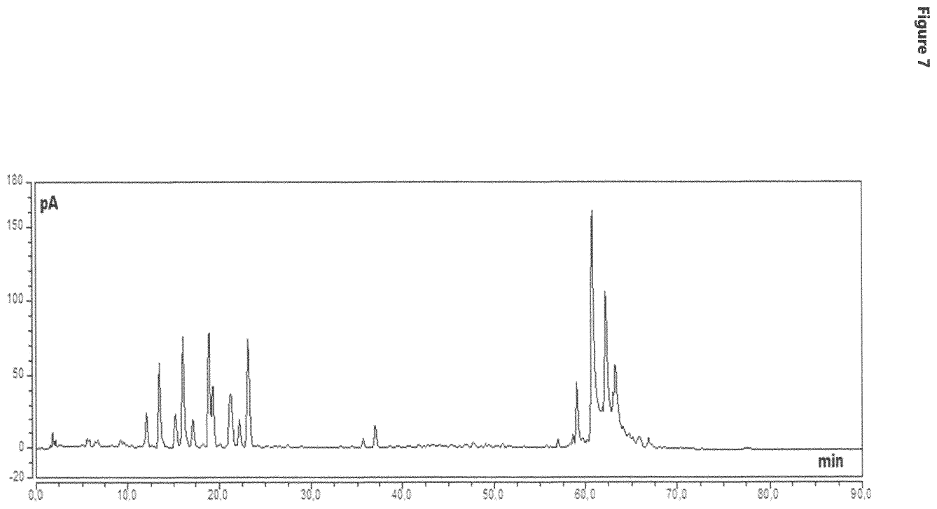

FIGS. 7 to 11 show comparative HPLC chromatograms (charge aerosol detection) of SFE extract versus supernatants 1-4.

FIG. 7 shows the SEE extract from example 3, 1 wt-% in THF/EtOH 7+3 (clear solution), injection volume: 5 μl.

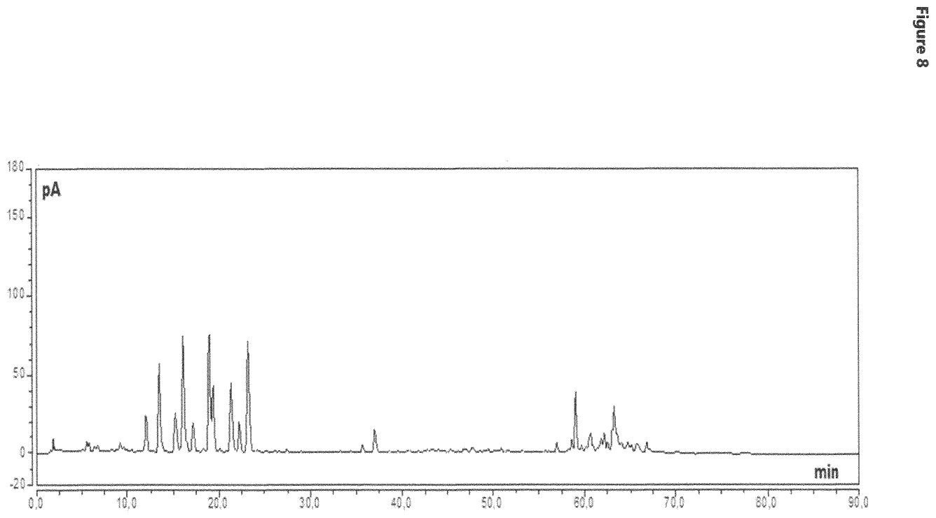

FIG. 8 shows the clear supernatant of 1 after filtration=0.9 wt-% in EtOH, injection volume: 5.5 μl.

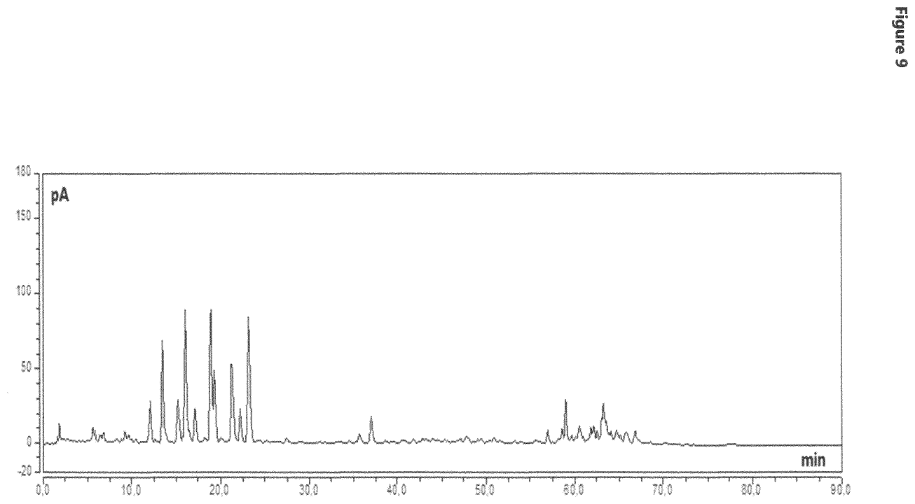

FIG. 9 shows the clear supernatant of 2 after filtration=2 wt-% in EtOH, injection volume: 2.5 μl.

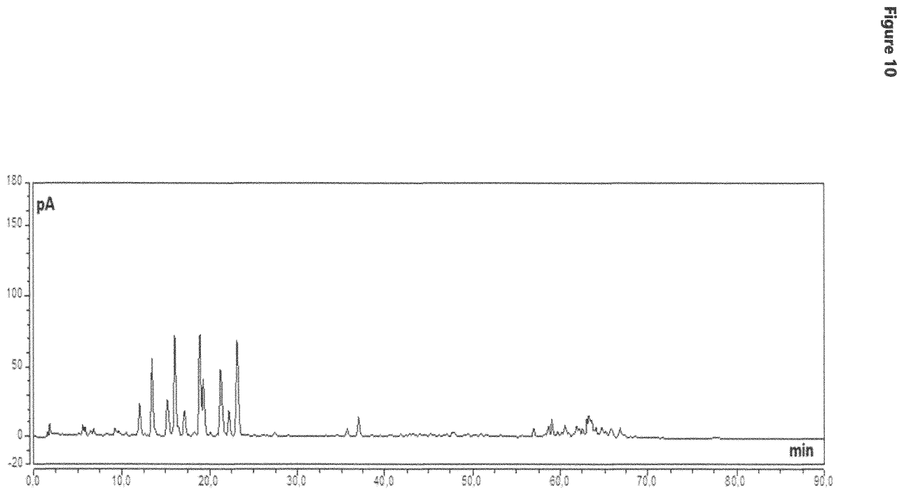

FIG. 10 shows the clear supernatant of 3 after filtration=5 wt-% in EtOH, 1:5 dilution in EtOH, injection volume: 5 μl.

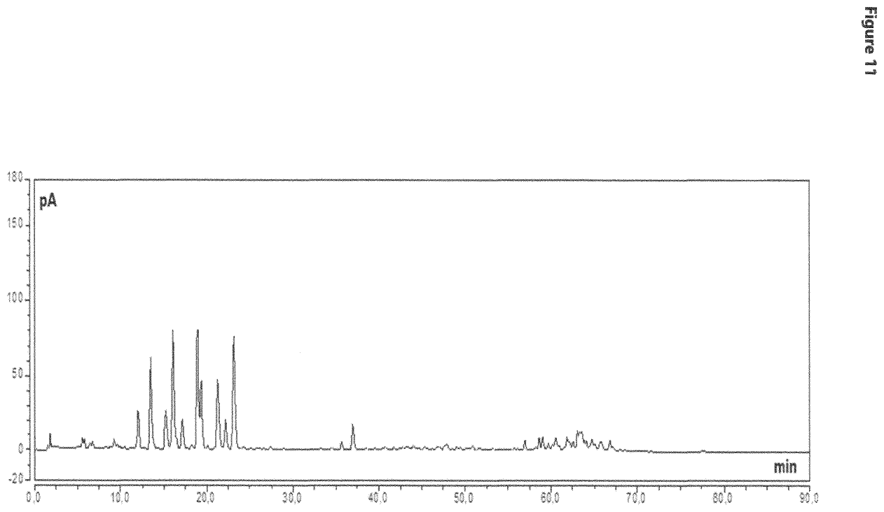

FIG. 11 shows the clear supernatant of 4 after filtration=10 wt-% in EtOH, 1:10 dilution in EtOH, injection volume: 5 μl.

When comparing the HPLC chromatograms at the same dry matter concentration obtained by adapting the injection volume or by dilution as given above, it is obvious that the group of the long chain alkenones eluting between 59 and 65 min is highly significantly decreased. The removal of this group can already be achieved at a low dry matter concentration such as e.g. 0.9 wt-% and as well, but even more pronounced at higher dry matter concentrations.

The quantitative determinations of the alkenones (table 24) verified the findings from the HPLC chromatograms (FIGS. 7 to 11). The content of alkenones is decreased from 11.6 to 1.7, 1.2, 0.7 and 0.5 wt-% respectively, corresponding to a reduction versus starting material, the T-ISO SFE extract, of 85, 90, 94 and 96% respectively.

By removing the alkenones from the extracts, content of free fatty acids and sterols are increased, as these compounds are well soluble in ethanol. The same effect is expected for glycerol bound fatty acids.

Dissolving of the dry matter of supernatant of 4 at 2 wt-% in vegetable oil triglyceride or hexyldecanol gave a clear solution already at ambient temperature without heating. No unsoluble parts or precipitation upon storage at ambient temperature or overnight at 5 to 8° C. was observed.

To compare the color of the 2 liquid mixtures, they were diluted with vegetable oil triglyceride (INCI name: Caprylic/Capric Triglyceride) to obtain an extract concentration of 0.018 wt.-%.

Color was measured by using the L*a*b* system as well as the Gardner value (Hach Lange Lico 690 instrument).

TABLE 25
Comparison of color

| Liquid mixture in | L* value | a* value | b* value | Gardner value |
|---|---|---|---|---|
| Vegetable oil | 100.3 | −1.0 | 2.3 | 0.1 |
| triglyceride | | | | |
| Hexyldecanol | 100.5 | −0.4 | 1.1 | 0.0 |

The results clearly show that the extract solutions are very light in color. The two obtained liquid mixtures are easy to formulate in different types of common cosmetic formulations.

### Example 11: Solid Mixture Containing T-ISO SFE Extract

A T-ISO SFE extract containing solid mixture was prepared using a liquid mixture prepared as described in example 5 with composition as given in table 26.

TABLE 26
Composition of solid mixture

| | Liquid mixture |
|---|---|
| Ingredient | wt-% |
| T-ISO CO2 SFE extract according | 6.0 |
| to example 2 | |
| Tocopherol | 0.6 |
| vegetable oil triglyceride (INCI | Ad 100 |
| name: Caprylic/Capric | |
| Triglyceride) | |

To prepare the emulsion for spray-drying, 226 g of maltodextrin DE17-20 from maize were dissolved in 432 g of water. 80 g of starch waxy maize capsul (INCI: Sodium Starch Octenylsuccinate) were added and dispersed. Afterwards, 100 g of the liquid mixture containing 6 wt-% of T-ISO CO2 SFE extract according to example 2 were added and the mixture was dispersed using a high-shear mixer. The dispersion was then spray-dried under standard conditions (supply air temperature: 190° C., exhaust air temperature: 90° C.) to obtain a powder with 25 wt-% loading of the T-ISO Co2 SFE extract liquid mixture.

TABLE 27
Composition of obtained powder

| | Solid mixture |
|---|---|
| Ingredient | wt-% |
| T-ISO CO2 SFE extract | 1.50 |
| according to example 2 | |
| Tocopherol | 0.15 |
| vegetable oil triglyceride (INCI | 23.35 |
| name: Caprylic/Capric | |
| Triglyceride) | |
| Maltodextrin | 55.39 |
| starch waxy maize capsul (INCI: | 19.61 |
| Sodium Starch | |
| Octenylsuccinate) | |

Formulation properties of the obtained powder was evaluated at 2 dosages in a W/O emulsion (soft cream) of the following formula:

TABLE 28
Composition soft cream

| | Raw Material | INCI | 1 | 2 |
|---|---|---|---|---|
| A. | Dragosan W/O P | Sorbitan Isostearate, Hydrogenated | 4.5 | 4.5 |
| | | Castor Oil, Ceresin, Beeswax (Cera Alba) | | |
| | Dehymuls PGPH | Polyglyceryl-2 Dipolyhydroxystearate | 1 | 1 |
| | Neo Heliopan BB | Benzophenone-3 | 0.5 | 0.5 |
| | Oxynex ST liquid | Diethylhexyl Syringylidenemalonate, | 0.5 | 0.5 |
| | | Caprylic/Capric Triglyceride | | |
| | Vegetable Oil Triglyceride | Caprylic/Capric/Triglyceride | 8 | 8 |
| | Vitamin E acetate | Tocpheryl Acetate | 1 | 1 |
| | Dragoxat 89 | Ethylhexyl Isononanoate | 10 | 10 |
| | Soybean Oil | Glycine Soja (Soybean) Oil | 5 | 5 |
| B. | Water, demin. | Water (Aqua) | Ad 100 | Ad 100 |
| | EDTA BD | Disodium EDTA | 0.1 | 0.1 |
| | SymOcide PS | Phenoxyethanol, Decylene Glycol, 1,2 | 1 | 1 |
| | | Hexanediol | | |
| | MgSO4 X 7 H2O | Magnesium Sulfate | 1 | 1 |
| | Glycerin 99% | Glycerin | 3 | 3 |
| | Solid mixture T-ISO SFE extract | Maltodextrin, Caprylic/Capric | 1 | 2 |
| | according to table 26 | Triglyceride, Isochrysis Galbana Extract | | |
| | Sum | | 100 | 100 |

Production method: Heat phase A and B separately to approximately 80° C. Add Phase B to A and emulsify using a vane stirrer. (10 min 400 U/min). Homogenize the emulsion using an Ultra Turrax stirrer for 1 m Ge at 7000 U/min.

The spray-dried powder was easy to incorporate in a cosmetic formulation. It is easy to handle and has good storage properties. Furthermore, it is preservative-free.

### Example 12: Ex Vivo Skin Pigmentation Increasing Efficacy of a Cosmetic Formulation Containing Liquid Mixtures of T-ISO Extracts

For evaluation of the ex vivo skin pigmentation increasing efficacy of another common type of cosmetic formulation, an oil in water emulsion containing a T-ISO SFE extract in form of the liquid mixture described in example 6 table 18 was used.

The following oil in water emulsions were prepared using this liquid mixture

TABLE 29
Oil in water emulsions

| | | 12.1 | 12.2 |
|---|---|---|---|
| | INCI | w/w % | w/w % |
| Phase A | | | |
| Dracorin ® GOC | Glyceryl Oleate Citrate, | 1.5 | 1.5 |
| | Caprylic/Capric | | |
| | Triglyceride | | |
| Vegetable oil | Caprylic/Capric | 6 | 6 |
| triglyceride | Triglyceride | | |
| PCL-Liquid 100 | Cetearyl Ethylhexanoate | 3 | 3 |
| Dragoxat ® 89 | Ethylhexyl Isononanoate | 3 | 3 |
| Abil 350 | Dimethicone | 0.3 | 0.3 |
| Phase B | | | |
| Aristoflex AVC | Ammonium | 1 | 1 |
| | Acryloyldimethyltaurate/VP | | |
| | Copolymer | | |
| Phase C | | | |
| Water | | Ad 100 | Ad 100 |
| SymOcide ® PS | Phenoxyethanol, | 1 | 1 |
| | Decylene Glycol, | | |
| | 1,2-Hexanediol | | |
| Hydrolite ® 5 green | Pentylene Glycol | 1 | 1 |
| Phase D | | | |
| Sodium Hydroxide 10% | Aqua, Sodium | 0.05 | 0.05 |
| solution | Hydroxide | | |
| Phase E | | | |
| Liquid mixture | | — | 1.5 |
| Sum | | 100.0 | 100.0 |

Production method: Heat Phase A and B separately to 70° C. Disperse Phase C in B. Add Phase BC to A and emulsify using an Ultra Turrax Stirrer. (3 min/6000 rpm). Allow to cool by using a vane stirrer (10 min/150 rpm). Add Phase D for neutralisation at approx. 40° C. Add Phase E to obtain formulation 12.2. The pH of 12.1 and 12.2 is 5,5-5,8.

The liquid mixture was easy to handle, well compatible with the formulation and no negative impact such as e.g. re-crystallization or modulation of odor was observed.

Organ culture of human skin was performed as described in example 1 to evaluate the skin pigmentation increasing efficacy of the 2 formulations versus untreated. Results are summarized in table 30.

TABLE 30
Comparison of mean pigmentation score untreated-
placebo-according to the invention

| | Mean | Modulation | Modulation |
|---|---|---|---|
| Test | pigmentation | vs control | vs 12.1 |
| sample | score | (untreated) | (placebo) |
| Untreated | 63.5 | — | |
| Formulation 12.1 | 61.4 | −3% | — |
| (Placebo) | | | |
| Formulation 12.2 | 73.9 | +16% | +20% |
| (1.5% liquid mixture) | | | |

The results clearly show that the liquid mixture used in an oil in water emulsion at 1.5% stimulates skin pigmentation versus untreated as well as versus placebo.

### Example 13: Reduction of Dihydroxyacetone-Induced Oxidative Stress

As T-ISO SFE extract according to example 7 exhibits antioxidant efficacy, its ability to reduce dihydroxyacetone-induced ROS and oxidative stress was evaluated.

Organ culture of human skin was performed starting from a skin sample, exciding pieces of approximately 8×3 mm (ø×thickness). Skin samples (6 per treatment) were cultured in an air-liquid interface in a perforated ring of stainless steel in contact with culture medium (modified Williams' E medium).

In the first step the effect of different dihydroxyacetone concentrations on ROS was investigated. Skin samples were cultivated overnight. On the next day, culture medium with 100 μM of 2′,7′-dichlorofluorescin diacetate (DCFH-DA, Sigma #D6883) was prepared and the culture medium was replaced by the medium containing DCFH-DA. Skin samples were then incubated for 30 minutes.

DCFH-DA is a cell permeant dye that measures ROS activity in the cell. After cell uptake, it is deacetylated by cellular esterases and later oxidized by the stimuli-induced ROS to 2′-7′dichlorofluorescein (DCF), a fluorescent reaction product which can be monitored by fluorescence-based microscopy.

After the 30 min of incubation with the DCFH-DA containing medium, the skin samples were washed in PBS buffer. Dihydroxacetone solutions of different concentrations in DMSO were topically applied and the skin samples were cultivated in culture medium in the incubator for 2 hours. As positive control, cumene hydroperoxide at 0.5 M topically applied was used. Afterwards, the skin samples were harvested, cryo-fixed and cut at the cryostat for image acquisition and analysis. The image acquisition was performed using Olympus BX51 microscope and Olympus DP70 camera. Two skin sections for each skin sample were cut and the related images were acquired and analysed. Doing so, 12 images were obtained for each formulation for analysis. Image analysis was performed within the dermis area selected from the upper part by following the perimeter of the basal lamina to the deep dermis. The obtained values from were normalized upon the dimension of the selected area.

Results are summarized in table 31.

TABLE 31
Mean ROS induction by dihydroxyacetone

| | Mean | Mean ROS |
|---|---|---|
| | fluorescence | induction vs |
| Sample | score | control (DMSO) |
| Control (DMSO) | 0.011 | 0% |
| 1% dihydroxyacetone | 0.013 | +24% |
| 5% dihydroxyacetone | 0.021 | +93% |
| 10% dihydroxyacetone | 0.042 | +293% |
| 50% dihydroxyacetone | 0.096 | +792% |
| Cumene hydroperoxide | 0.775 | +7145% |

The results clearly show a dose-dependent induction of ROS (mean fluorescent score) although at a significantly lower level than induced by 0.5 M cumene hydroperoxide.

In the second step, cosmetic formulations as given in table 32 were used to investigate the anti-oxidant effect of T-ISO extract and its ability to reduce the dihydroxyacetone induced ROS.

TABLE 32
Cosmetic formulations

| | | 13.1 | 13.2 | 13.3 | 13.4 | 13.5 |
|---|---|---|---|---|---|---|
| | INCI | w/w % | w/w % | w/w % | w/w % | w/w % |
| Phase A | | | | | | |
| Water | | Ad 100 | Ad 100 | Ad 100 | Ad 100 | Ad 100 |
| Hydrolite ® 5 | Pentyleneglycol | 1.0 | 1.0 | 1.0 | 1.0 | 1.0 |
| Phase B | | | | | | |
| PCL-Liquid 100 | Cetearyl | 3.0 | 3.0 | 3.0 | 3.0 | 3.0 |
| | Ethylhexanoate | | | | | |
| Lanette O | Cetearyl Alcohol | 2.0 | 2.0 | 2.0 | 2.0 | 2.0 |
| Mineral Oil 5° E | Mineral Oil | 3.0 | 3.0 | 3.0 | 3.0 | 3.0 |
| Eutanol G | Octyldodecanol | 4.0 | 4.0 | 4.0 | 4.0 | 4.0 |
| Abil 350 | Dimethicone | 0.5 | 0.5 | 0.5 | 0.5 | 0.5 |
| Phase C | | | | | | |
| Pemulen TR-1 | Acrylates/C10-30 Alkyl | 0.2 | 0.2 | 0.2 | 0.2 | 0.2 |
| | Acrylate Crosspolymer | | | | | |
| Carbopol Ultrez-21 | Acrylates/C10-30 Alkyl | 0.05 | 0.05 | 0.05 | 0.05 | 0.05 |
| Polymer | Acrylate Crosspolymer | | | | | |
| Phase D | | | | | | |
| Sodium Hydroxide | Aqua, Sodium | 0.5 | 0.5 | 0.5 | 0.5 | 0.5 |
| 10% solution | Hydroxide | | | | | |
| Phase E | | | | | | |
| Hydrolite ® 5 | Pentyleneglycol | 1.0 | 1.0 | 1.0 | 1.0 | 1.0 |
| Liquid mixture | | — | 1.0 | 2.0 | — | — |
| Tocopherol | Tocopherol | — | — | — | 0.005 | 0.01 |
| Sum | | 100.0 | 100.0 | 100.0 | 100.0 | 100.0 |

Production method: Heat phase A and B separately to 70° C. Disperse Phase C in B. Add Phase BC to A and emulsify using an Ultra Turrax stirrer. Allow to cool by using a vane stirrer. Add Phase D for neutralisation at approx. 40° C. The liquid mixture (13.2 and 13.3) and tocopherol (13.4 and 13.5) were presolved in Hydrolite® 5 and were added to the formulation 13.1 by using a vane stirrer. pH value is adjusted to 5.5-5.8.

The liquid mixture was composed according to table 18 and contained T-ISO SFE extract (0.9%) and tocopherol (0.5%). It was used at 1% (13.2) and 2% (13.3). As tocopherol is a well known antioxidant, formulations 14.3 (0.005% tocopherol corresponding to the tocopherol content in 13.2) and 13.5 (0.01% tocopherol corresponding to the tocopherol content in 13.3) were prepared and tested in parallel. Target was to verify if the observed effect was alone or at least partly due to the T-ISO SFE extract and not due to the tocopherol alone.

At start of the skin culture, the formulations 13.1 to 13.5 were applied topically on the skin samples and afterwards they were cultivated overnight. On the next day, the topical treatments with formulations 13.1 to 13.5 were renewed for 1 hour. In parallel, culture medium with 100 μM of 2′,7′-dichlorofluorescin diacetate (DCFH-DA, Sigma #D6883) was prepared. After the 1 hour of incubation, the culture medium was replaced by the medium containing DCFH-DA and the skin samples were incubated for 30 minutes.

Afterwards, the skin samples were washed in PBS buffer. Dihydroxyacetone treatment, skin preparation for image acquisition followed by image analysis was performed as described above.

Results are summarized in table 33.

TABLE 33
Comparison of mean ROS reduction score of formulations 13.1. to 13.5

| | | | | | Modulation vs |
|---|---|---|---|---|---|
| | | | | Modulation | respective |
| | | | | vs 13.1 | tocopherol |
| | | | Modulation | (placebo) + | placebo + |
| | | Modulation | vs 50% | 50% | 50% |
| | Mean | vs control | dihydroxy- | dihydroxy- | dihydroxy- |
| Test sample | score | (DMSO) | acetone | acetone | acetone |
| Control (DMSO) | 0.216 | 0% | | | |
| 50% | 0.691 | +220% | 0% | | |
| dihydroxyacetone | | | | | |
| Formulation 13.1 | 0.556 | | −20% | 0% | |
| (Placebo) + 50% | | | | | |
| dihydroxyacetone | | | | | |
| Formulation 13.2 | 0.365 | | −47% | −34% | −36% vs 14.4 |
| (1% liquid mixture) + | | | | | |
| dihydroxyacetone | | | | | |
| Formulation 13.3 | 0.312 | | −55% | −44% | −32% vs 14.5. |
| (2% liquid mixture) + | | | | | |
| dihydroxyacetone | | | | | |
| Formulation 13.4 | 0.566 | | −18% | +2% | 0% |
| (0.005% tocopherol) + | | | | | |
| dihydroxyacetone | | | | | |
| Formulation 13.5 | 0.457 | | −34% | −18% | 0% |
| (0.01% tocopherol) + | | | | | |
| dihydroxyacetone | | | | | |

The results clearly verified that 50% dihydroxyacetone induced ROS and oxidative stress (+220% vs control). The placebo 13.1 reduced the dihydroxyacetone-induced ROS level by 20%. Versus the placebo 13.1, 0.005% tocopherol (13.4.) had no relevant effect (+2%) and 0.01% tocopherol (14.5.) had a moderate effect (−18%) on the dihydroxyacetone-induced ROS level. A clearly stronger reduction of dihydroxyacetone-induced ROS level versus placebo (13.1) was observed for 1% (13.2) and 2% (13.3) of the liquid mixture.

Comparison versus the respective (same concentration) tocopherol containing formulation clearly proved that the observed effect is mainly due to the contained T-ISO extract. Formula 13.2 decreased the ROS level versus 13.4 by 36% and formula 13.3 decreased the ROS level versus 13.5 by 32%.

Therefore, combination of the biological, slower tanning T-ISO extract with the chemical, faster browning dihydroxyacetone is especially advantageous as it is not only expected to lead to a visually perceivable tan already in much shorter time but it also leads to a lower ROS induction in the skin as the T-ISO extract partly counteracts the dihydroxyacetone-induced oxidative stress.

### Example 14: Reduction of UV-Induced Oxidative Stress

As T-ISO SFE extract according to example 7 and 13 exhibits antioxidant efficacy, its ability to reduce UVA-induced oxidative stress was evaluated. Tocopherol acetate (Sigma, T3376) was used as positive control.

Organ culture of human skin was performed starting from a skin sample, exciding pieces of approximately 8×3 mm (ø×thickness). Skin samples (6 per treatment) were cultured in an air-liquid interface in a perforated ring of stainless steel in contact with culture medium (modified Williams' E medium).

Cosmetic formulations as given in table 34 were used.

TABLE 34
Cosmetic formulations

| | | 14.1 | 14.2 | 14.3 |
|---|---|---|---|---|
| | INCI | w/w % | w/w % | w/w % |
| Phase A | | | | |
| Water | | Ad 100 | Ad 100 | Ad 100 |
| Hydrolite ® 5 | Pentyleneglycol | 1.0 | 1.0 | 1.0 |
| Phase B | | | | |
| PCL-Liquid 100 | Cetearyl | 3.0 | 3.0 | 3.0 |
| | Ethylhexanoate | | | |
| Lanette O | Cetearyl Alcohol | 2.0 | 2.0 | 2.0 |
| Mineral Oil | Mineral Oil | 3.0 | 3.0 | 3.0 |
| 5° E | | | | |
| Eutanol G | Octyldodecanol | 4.0 | 4.0 | 4.0 |
| Abil 350 | Dimethicone | 0.5 | 0.5 | 0.5 |
| Phase C | | | | |
| Pemulen TR-1 | Acrylates/C10-30 Alkyl | 0.2 | 0.2 | 0.2 |
| | Acrylate Crosspolymer | | | |
| Carbopol | Acrylates/C10-30 Alkyl | 0.05 | 0.05 | 0.05 |
| Ultrez-21 | Acrylate Crosspolymer | | | |
| Polymer | | | | |
| Phase D | | | | |
| Sodium | Aqua, Sodium | 0.5 | 0.5 | 0.5 |
| Hydroxide | Hydroxide | | | |
| 10% solution | | | | |
| Phase E | | | | |
| Hydrolite ® 5 | Pentyleneglycol | 1.0 | 1.0 | 1.0 |
| Liquid mixture | | — | 1.0 | 2.0 |
| Sum | | 100.0 | 100.0 | 100.0 |

Production method: Heat phase A and B separately to 70° C. Disperse Phase C in B. Add Phase BC to A and emulsify using an Ultra Turrax stirrer. Allow to cool by using a vane stirrer. Add Phase D for neutralisation at approx. 40° C. The liquid mixture was presolved in Hydrolite® 5 and was added to formulation 14.1 under stirring. pH value is adjusted to 5.5-5.8.

The liquid mixture was composed according to table 18.

At start of the skin culture, in parallel to untreated, the formulations 14.1 to 14.3 and 100% tocopherol acetate were applied topically on the skin samples and afterwards they were cultivated overnight. On the next day, the topical treatments were renewed for 1 hour. In parallel, culture medium with 100 μM of 2′,7′-dichlorofluorescin diacetate (DCFH-DA, Sigma #D6883) was prepared. After 1 hour of incubation of the skin samples, the culture medium was replaced by the medium containing DCFH-DA and the skin samples were incubated for 30 minutes. Then, the skin samples were washed in PBS buffer and UVA irradiation (lamp: Bio Sun by Vilber Lourmat, 60 J/cm2) was performed. Afterwards, the skin samples were harvested, cryo-fixed and cut at the cryostat for image acquisition and analysis. The image acquisition was performed using Olympus BX51 microscope and Olympus DP70 camera. Two skin sections for each skin sample were cut and the related images were acquired and analysed. Doing so, 12 images were obtained for each formulation for analysis. Image analysis was performed within the dermis area selected from the upper part by following the perimeter of the basal lamina to the deep dermis. The obtained values from were normalized upon the dimension of the selected area.

Results are summarized in table 35.

TABLE 35
Comparison of mean fluorescence score

| | | Modulation vs | Modulation vs | Modulation |
|---|---|---|---|---|
| | Mean ROS score | untreated no | untreated + | vs placebo |
| Test sample | score | UVA | UVA | (14.1) + UVA |
| Untreated no UVA | 0.015 | — | | |
| Untreated + UVA | 0.573 | +3623% | — | — |
| 100% Tocopherol | 0.157 | — | −73% | — |
| acetate (positive | | | | |
| control) | | | | |
| Formulation 14.1 | 1.587 | — | +177% | — |
| (Placebo) + UVA | | | | |
| Formulation 14.2 | 0.335 | — | −42% | −79% |
| (1% liquid mixture) + | | | | |
| UVA | | | | |
| Formulation 14.3 | 0.272 | — | −53% | −83% |
| (2% liquid mixture) + | | | | |
| UVA | | | | |

As expected, comparison of UVA-irradiated untreated skin samples versus non-UVA-irradiated untreated skin samples proved a significant increase in fluorescence score, i.e. ROS (+3623%).

The positive control 100% tocopherol acetate exhibited as expected a significant reduction of UVA-induced ROS vs untreated UVA-irradiated by 73%.

The placebo formulation 14.1 even increased the UVA-induced ROS whereas the two formulations containing the liquid mixture at 1 and 2% dose-dependently decreased the UVA-induced ROS level versus untreated UVA-irradiated as well as placebo treated UVA-irradiated.

The results clearly show that the T-ISO extract containing liquid mixture used in a cosmetic formulation at 1 and 2% also reduces UV-induced ROS induction and oxidative stress and can therefore be expected to also clearly decrease ROS and oxidative stress associated tissue damages. Therefore, a combination with UV filters (organic and/or inorganic ones) can be advantageous as it helps on the one hand to reduce ROS formation and on the other hand increases UV-independent skin pigmentation.

### Example 15: Formulation Examples

In formulations 1 to 22 the following perfume oils PF01 to PF03 were each used as fragrance (DPG=dipropylene glycol).

TABLE 36
Composition of perfume oil 1 (PO1, Amounts in ‰ b.w.)

| Ingredients | Amount |
|---|---|
| ALDEHYDE C14 SO-CALLED | 2 |
| ALLYL AMYL GLYCOLATE 10% DPG | 5 |
| ANISIC ALDEHYDE PURE | 5 |
| APPLE OLIFFAC TYPE | 10 |
| BENZYLACETATE | 50 |
| BERGAMOT IDENTOIL ® COLOURLESS | 15 |
| CANTHOXAL | 5 |
| CETALOX 10% IPM | 3 |
| CITRONELLOL 950 | 40 |
| DAMASCENONE TOTAL 1% DPG | 5 |
| DAMASCONE ALPHA 10% DPG | 5 |
| DAMASCONE DELTA 10% DPG | 2 |
| DIMETHYL BENZYL CARBINYL BUTYRATE | 2 |
| DIPROPYLENE GLYCOL | 178 |
| EBANOL | 2 |
| ETHYL DECADIENOATE TRANS CIS-2,4 10% IPM | 2 |
| FLOROSA | 5 |
| FRAMBINON ® 10% DPG | 7 |
| GALAXOLIDE 50% IN IPM | 100 |
| GALBEX TYPE BASE | 1 |
| GERANYL ACETATE PURE | 2 |
| HEDIONE | 30 |
| HELIOTROPIN | 10 |
| HEXENYL ACETATE CIS-3 10% DPG | 1 |
| HEXENYL SALICYLATE CIS-3 | 5 |
| HEXYL CINNAMIC ALDEHYDE ALPHA | 70 |
| HEXYL SALICYLATE | 50 |
| HYDROXY CITRONELLAL | 10 |
| ISO E SUPER | 15 |
| ISORALDEINE 70 | 20 |
| LEAFOVERT ® | 1 |
| LILIAL | 60 |
| LINALOOL | 60 |
| LINALYL ACETATE | 20 |
| LYRAL | 7 |
| MANZANATE | 2 |
| PHENOXANOL | 7 |
| PHENYLETHYL ALCOHOL | 120 |
| SANDAL MYSORE CORE | 2 |
| SANDRANOL ® | 7 |
| STYRALYL ACETATE | 3 |
| TAGETES RCO 10% TEC | 2 |
| TERPINEOL PURE | 20 |
| TETRAHYDROGERANIOL 10% DPG | 5 |
| TONALIDE | 7 |
| VERTOCITRAL 10% DPG | 5 |
| VERTOFIX | 15 |
| Total | 1000 |

TABLE 37
Composition of perfume oil 2 (PO2, Amounts in ‰ b.w.)

| Ingredients | Amount |
|---|---|
| Acetophenone, 10% in DPG | 10 |
| n-Undecanal | 5 |
| Aldehyde C14, so-called (peach aldehyde) | 15 |
| Allylamyl glycolate, 10% in DPG | 20 |
| Amyl salicylate | 25 |
| Benzyl acetate | 60 |
| Citronellol | 80 |
| d-Limonene | 50 |
| Decenol trans-9 | 15 |
| Dihydromyrcenol | 50 |
| Dimethylbenzylcarbinyl acetate | 30 |
| Diphenyloxide | 5 |
| Eucalyptol | 10 |
| Geraniol | 40 |
| Nerol | 20 |
| Geranium oil | 15 |
| Hexenol cis-3, 10% in DPG | 5 |
| Hexenyl salicylate cis-3 | 20 |
| Indole, 10% in DPG | 10 |
| Alpha-ionone | 15 |
| Beta-ionone | 5 |
| Lilial ® (2-methyl-3-(4-tert-butyl-phenyl)propanal) | 60 |
| Linalool | 40 |
| Methylphenyl acetate | 10 |
| Phenylethyl alcohol | 275 |
| Styrolyl acetate | 20 |
| Terpineol | 30 |
| Tetrahydrolinalool | 50 |
| Cinnamyl alcohol | 10 |
| Total: | 1000 |

TABLE 38
Composition of perfume oil 3 (PO3, Amounts in ‰ b.w.)

| Ingredients | Amount |
|---|---|
| AMBRETTOLIDE (MACRO) | 10 |
| AMBROXIDE 10% in IPM | 10 |
| BENZYL ACETATE | 20 |
| BENZYL SALICYLATE | 15 |
| BERGAMOT OIL. bergapten-free | 60 |
| CALONE ® 1951 10% in DPG | 15 |
| COUMARIN | 5 |
| CYCLOGALBANATER 10% in DPG | 10 |
| ALPHA -DAMASCONE 1% in DPG | 20 |
| DIHYDROMYRCENOL | 10 |
| ETHYL LINALOOL | 75 |
| ETHYL LINALYLACETATE | 50 |
| ETHYL MALTOL 1% in DEP | 10 |
| ETHYLENE BRASSYLATE (MACRO) | 80 |
| FLOROSA | 40 |
| GERANYLACETATE | 10 |
| HEDIONE ® HC/30 | 35 |
| HEDIONE ® | 210 |
| HELIONAL ® | 15 |
| HELVETOLIDE ® (ALICYC) | 30 |
| HEXENYLSALICYLATE CIS-3 | 20 |
| ISO E SUPER ® | 40 |
| LEAFOVERT ® 10% in DEP | 10 |
| LILIAL ® | 80 |
| LYRAL ® | 20 |
| MANDARIN OIL | 10 |
| STYRALYL ACETATE | 5 |
| SYMROSE ® | 15 |
| VANILLIN 10% in DEP | 20 |
| DIPROPYLENE GLYCOL (DPG) | 50 |
| TOTAL | 1000 |

TABLE 39
Cosmetic formulations 1 to 11 (amounts in parts b.w.)

| Ingredients INCI name | 1 | 2 | 3 | 4 | 5 | 6 | 7 | 8 | 9 | 10 | 11 |
|---|---|---|---|---|---|---|---|---|---|---|---|
| T-ISO SFE extract (0.9 wt-%) | 2.0 | | 4.0 | 2.0 | | | 3.0 | 4.0 | | | |
| containing liquid mixture | | | | | | | | | | | |
| using vegetable oil | | | | | | | | | | | |
| triglyceride (Caprylic/Capric | | | | | | | | | | | |
| Triglyceride) as described in | | | | | | | | | | | |
| Example 5 | | | | | | | | | | | |
| T-ISO SFE extract (2.0 wt-%) | | 1.0 | | | | 4.0 | | | 2.0 | | |
| containing liquid mixture | | | | | | | | | | | |
| using soybean oil (Glycine | | | | | | | | | | | |
| Soja (Soybean) Oil) as | | | | | | | | | | | |
| described in Example 5 | | | | | | | | | | | |
| T-ISO SFE extract (2.0 wt-%) | | | | | | | | | | | 1.0 |
| containing liquid mixture | | | | | | | | | | | |
| using hexyldecanol (Cetyl | | | | | | | | | | | |
| Alcohol) as described in | | | | | | | | | | | |
| Example 10 | | | | | | | | | | | |
| T-ISO SFE extract (1.5 wt-%) | | | | | 1.0 | | | | | 0.2 | |
| containing solid mixture using | | | | | | | | | | | |
| maltodextrin as described in | | | | | | | | | | | |
| Example 11 | | | | | | | | | | | |
| Acusol OP 301 | | | | | | | 1.0 | | | | |
| Styrene/Acrylates Copolymer | | | | | | | | | | | |
| Actipone Laminaria | | | | | | | | | | | 1.0 |
| Saccharina GW Glycerin, | | | | | | | | | | | |
| Aqua, Laminaria Saccharina | | | | | | | | | | | |
| Extract | | | | | | | | | | | |
| Aloe Vera Gel Concentrate | | | | | 0.5 | | | | | | |
| 10/1 Aloe Barbadensis Leaf | | | | | | | | | | | |
| Juice | | | | | | | | | | | |
| Antil 120 Plus PEG-120 | | | | | | | 1.0 | | | | |
| Methyl Glucose Dioleate | | | | | | | | | | | |
| Argan Oil Argania Spinosa | | | | | | | | | | | 1.0 |
| Kernel Oil | | | | | | | | | | | |
| Avocado Oil CA | | | | | | | | | | | 1.0 |
| Persea Gratissima Oil | | | | | | | | | | | |
| Biotive L-Arginine Arginine | | | | | | | | | | | 1.0 |
| Butylene Glycol Butylene | | | | | | | | | | 3.0 | |
| Glycol | | | | | | | | | | | |
| Candelilla Wax | | | | | | | | 10.0 | | | |
| Euphorbia Cerifera Cera | | | | | | | | | | | |
| Carbopol Ultrez 10 Polymer | | 0.4 | | | 0.45 | | | | | | |
| Carbomer | | | | | | | | | | | |
| Carbopol Ultrez 21 Polymer | | | | | | | | | | | 0.4 |
| Acrylates/C10-30 Alkyl | | | | | | | | | | | |
| Acrylate Crosspolymer | | | | | | | | | | | |
| Cell-U-Lash 40 Cellulose | | | | | | | | 10.0 | | | |
| Cetiol CC Dicaprylyl | 20.0 | | | | | | | | | | |
| Carbonate | | | | | | | | | | | |
| Cetiol Ultimate Tridecane, | | | | | | | | | | | 4.0 |
| Undecane | | | | | | | | | | | |
| Citric acid 10% solution | | | 0.02 | | | | | | | | |
| Aqua, Citric acid | | | | | | | | | | | |
| Citric acid 20% solution | | | | | | 0.42 | 0.25 | | | | |
| Aqua, Citric acid | | | | | | | | | | | |
| Color | | | | | | | | | 0.4 | | |
| Color (aqueous solution of 2% | | | | 0.15 | | | | | | | |
| D&C Red 33 (656855) - | | | | | | | | | | | |
| C.I. 17200) | | | | | | | | | | | |
| Colorona Oriental Beige | | | | | | | | 0.3 | | | |
| Mica, Titanium Dioxide, Iron | | | | | | | | | | | |
| Oxides (CI 77491) | | | | | | | | | | | |
| Comperlan IP Cocamide | | | | | | | 1.0 | | | | |
| MIPA | | | | | | | | | | | |
| Corapan TQ Diethylhexyl 2,6- | | | | | | | | | | | 1.0 |
| naphthalate | | | | | | | | | | | |
| Cutina GMS V Glyceryl | | | 2.0 | 2.5 | | | | | | | |
| Stearate | | | | | | | | | | | |
| Dehyton PK 45 | | | | | | | 8.0 | | | | |
| Cocamidopropoyl Betaine | | | | | | | | | | | |
| Dihydroxyacetone | | | 3.0 | | | | | | | | |
| Dihydroxyacetone | | | | | | | | | | | |
| Dimethicone Dimethicone | | 0.5 | 0.5 | 1.0 | | | | | | | |
| Dracorin 100 SEP Glyceryl | | | 3.0 | | | | | | | | |
| Stearate, PEG-100 Stearate | | | | | | | | | | | |
| Dracorin GOC Glyceryl | | 4.0 | | | | 3.0 | | 1.0 | | | |
| Oleate Citrate, Caprylic/Capric | | | | | | | | | | | |
| Triglyceride | | | | | | | | | | | |
| DragoCalm Aqua, Glycerin, | | | | | | | | | | 1.5 | |
| Avena Sativa Kernel Extract | | | | | | | | | | | |
| Dragosantol 100 Bisabolol | | | | 0.1 | | | | | | 0.1 | |
| Dragosine Carnosine | | | | | 0.1 | | | | | 0.1 | |
| Dragoxat 89 Ethylhexyl | | | | 5.0 | | | | | 5.2 | | 2.0 |
| Isononanoate | | | | | | | | | | | |
| Edeta BD Disodium EDTA | | 0.1 | | 0.05 | 0.1 | | 0.1 | | | | 0.1 |
| Emulsiphos Potassium Cetyl | | | | 2.5 | | | | | | | 2.5 |
| Phosphate, Hydrogenated | | | | | | | | | | | |
| Palm Glycerides | | | | | | | | | | | |
| Estearina L2SM GS Stearic | | | | | | | 0.5 | | | | |
| Acid, Palmitic Acid | | | | | | | | | | | |
| Ethanol 96% Alcohol Denat. | | | | | | | | | Ad 100 | | |
| Extrapone Almond Milk N | | | 1.0 | | | | | | | | |
| Prunus Amygdalus Dulcis Seed | | | | | | | | | | | |
| Extract, Glycerin, Aqua, | | | | | | | | | | | |
| Propylene Glycol, Pentylene | | | | | | | | | | | |
| Glycol, PEG-40 Hydrogenated | | | | | | | | | | | |
| Castor Oil, Trideceth-9, | | | | | | | | | | | |
| PrunusAmygdalus Dulcis Oil, | | | | | | | | | | | |
| Decylene Glycol, 1,2- | | | | | | | | | | | |
| Hexanediol, Sine Adipe Lac | | | | | | | | | | | |
| Extrapone Corallina | | | | 0.1 | | | | | | | |
| Officinalis GW CL Glycerin, | | | | | | | | | | | |
| Aqua, Hydrolyzed | | | | | | | | | | | |
| Corallina Officinalis | | | | | | | | | | | |
| Extrapone Cucumber CL | | | | | | | | | | 1.0 | |
| Aqua, Propylene Glycol, | | | | | | | | | | | |
| Cucumis sativus Juice | | | | | | | | | | | |
| Extrapone Matcha GW | | | 1.0 | | | | | | | | |
| Aqua, Glycerin, | | | | | | | | | | | |
| Camellia Sinensis | | | | | | | | | | | |
| Leaf Powder, Xanthan Gum | | | | | | | | | | | |
| Fragrance Parfum | 1.0 | 0.5 | 0.3 | 1.0 | 0.4 | 2.0 | 1.5 | 0.3 | 0.3 | 0.05 | 0.5 |
| Frescolat ML Cryst Menthyl | | | | | | | | | | 0.3 | |
| Lactate | | | | | | | | | | | |
| Frescolat X-Cool Menthyl | | | | 0.5 | | | | | 1.0 | | |
| Ethylamido Oxalate | | | | | | | | | | | |
| Glucotain Care Cocoyl | | | | | | | 2.0 | | | | |
| Methyl Glucamide | | | | | | | | | | | |
| Glycerin Glycerin | | | 3.0 | 3.0 | 5.0 | | | | | 2.0 | |
| Guar Gum Guar Gum | | | | | | | | | | 0.1 | |
| HDK-N20D Silica | | | | | | | | 1.0 | | | |
| Hemp Oil Cannabis Sativa | | | | | | 2.5 | | | | | |
| Seed Oil | | | | | | | | | | | |
| Hydrolite 5 Pentylene Glycol | | | | 1 | 5.0 | | | | 3.0 | | |
| Hydrolite 5 green Pentylene | 2.0 | 2.0 | 3.0 | | | | | | | 3.5 | |
| Glycol | | | | | | | | | | | |
| Hydrolite 6 1,2-Hexanediol | | | | | | 1.0 | | | | | |
| Hydroviton 24 Aqua, | | | | 1.5 | | | | | | | |
| Pentylene Glycol, Glycerin, | | | | | | | | | | | |
| Lactic Acid, Sodium Lactate, | | | | | | | | | | | |
| Serine, Urea, Sorbitol, Sodium | | | | | | | | | | | |
| Chloride, Allantoin | | | | | | | | | | | |
| Hydroviton ® PLUS 2290 | | | | | | | | | | 2.0 | |
| Aqua, Pentylene Glycol, | | | | | | | | | | | |
| Glycerin, Fructose, Urea, Citric | | | | | | | | | | | |
| acid, Sodium hydroxide, | | | | | | | | | | | |
| Maltose, Sodium PCA, Sodium | | | | | | | | | | | |
| Chloride, Sodium Lactate, | | | | | | | | | | | |
| Trehalose, Allantoin, Sodium | | | | | | | | | | | |
| hyaluronate, Glucose | | | | | | | | | | | |
| Isoadipate Diisopropyl | | 3.0 | 5.0 | | | | | 1.5 | | | 3.0 |
| Adipate | | | | | | | | | | | |
| Isodragol Triisononanoin | 17.5 | 2.0 | | | | | | 3.0 | | | |
| Isopropyl Palmitate | Ad 100 | | | | | | | | | | |
| Isopropyl palmitate | | | | | | | | | | | |
| Jojoba Oil Simmondsia | | | | | | | | | | | 1.0 |
| Chinensis Seed Oil | | | | | | | | | | | |
| Kahlwax 6290 Berry Wax | | | | | | | | 1.0 | | | |
| Rhus Verniciflua Peel Cera | | | | | | | | | | | |
| Kahlwax 6642 Carnauba | | | | | | | | 9.5 | | | |
| Wax LC organic | | | | | | | | | | | |
| Copernicia Cerifera Cera | | | | | | | | | | | |
| Keltrol CG-T Xanthan Gum | | | | 0.2 | 0.3 | | | | | 0.1 | 0.2 |
| Lanette 16 Cetyl alcohol | | | | | | | | | | | 1.0 |
| Lanette 22 Behenyl Alcohol | | | 2.0 | | | | | | | | |
| Lanette O Cetearyl Alcohol | | 2.5 | 2.0 | 2.0 | | | 2.0 | | | | 3.5 |
| Lipocire A Hydrogenated | | | | 2.0 | | | | | | | |
| Palm Kernel Glycerides, | | | | | | | | | | | |
| Hydrogenated Palm Glycerides | | | | | | | | | | | |
| Macadamia Oil Macadamia | | | | | | | | | | | 0.5 |
| Integrifolia Seed Oil | | | | | | | | | | | |
| Nativacare 5600 Zea Corn | | | | | | | | | | 2.0 | |
| Starch | | | | | | | | | | | |
| Neo-Heliopan 303 | | | | | | | | 8.0 | 10.0 | | |
| Octocrylene | | | | | | | | | | | |
| Neo-Heliopan 357 Butyl | | | | | | | | 3.0 | 5.0 | | 2.5 |
| Methoxydibenzoylmethane | | | | | | | | | | | |
| Neo-Heliopan BMT Bis- | | | | | | | | | | | 1.0 |
| ethylhexyloxyphenol | | | | | | | | | | | |
| methoxyphenyl triazine | | | | | | | | | | | |
| Neo-Heliopan HMS | | | | | | | | 5.0 | 10.0 | | |
| Homosalate | | | | | | | | | | | |
| Neo-Heliopan Hydro | | | | | | | | | | | 2.0 |
| Phenylbenzimidazole sulfonic | | | | | | | | | | | |
| acid | | | | | | | | | | | |
| Neo-Heliopan OS Ethylhexyl | | | | | | | | 5.0 | 5.0 | | 5.0 |
| Salicylate | | | | | | | | | | | |
| Oxynex ST Liquid | | | | | 0.5 | | | | | | |
| Diethylhexyl | | | | | | | | | | | |
| Syringylidenemalonate, | | | | | | | | | | | |
| Caprylic/Capric Triglyceride | | | | | | | | | | | |
| Panthenol Panthenol | | | | | | 0.5 | | | | 0.5 | |
| PCL Liquid Cetearyl | 12.0 | 3.0 | 5.0 | | | | | | | | |
| Ethylhexanoate, Isopropyl | | | | | | | | | | | |
| Myristate | | | | | | | | | | | |
| PCL Liquid 100 Cetearyl | | | | | | 3.5 | | 11.7 | | | |
| Ethylhexanoate | | | | | | | | | | | |
| Phenoxyethanol | | | | 0.5 | | | | | | | |
| Phenoxyethanol | | | | | | | | | | | |
| Phytoconcentrole Carrot | 1.0 | | | | | | | | | | |
| Helianthus Annuus Seed Oil, | | | | | | | | | | | |
| Beta-Carotene, Daucus Carota | | | | | | | | | | | |
| Sativa Root Extract | | | | | | | | | | | |
| Phytoconcentrole Seaweed | | | | | | | | | 1.0 | | |
| Glycine Soja Oil, | | | | | | | | | | | |
| Caprylic/Capric Triglyceride, | | | | | | | | | | | |
| Fucus Vesiculosus Extract | | | | | | | | | | | |
| Protelan AGL Sodium Lauroyl | | | | | | 1.5 | | | | | |
| Glutamate | | | | | | | | | | | |
| Sebumol S 1000 MIPA- | | | | | | Ad 100 | | | | | |
| Cocoyl Sarcosinate, | | | | | | | | | | | |
| Polyglyceryl-3 Polyricinoleate, | | | | | | | | | | | |
| Polyglyceryl-3 Diisostearate, | | | | | | | | | | | |
| Polyglyceryl-3 Caprate | | | | | | | | | | | |
| Sepimax ZEN Polyacrylate | | | 1.0 | | | | | | | | |
| Crosspolymer-6 | | | | | | | | | | | |
| Sepinov EMT 10 | | | | | 0.5 | | | | | | |
| Hydroxyethyl Acrylate/Sodium | | | | | | | | | | | |
| Acryloyldimethyl Taurate | | | | | | | | | | | |
| Copolymer | | | | | | | | | | | |
| Shea Butter (Organic) | | | | 2.0 | | | | 5.0 | | | 2.0 |
| Butyrospermum Parkii Butter | | | | | | | | | | | |
| Simulgel EG | | | | 1.0 | | | | | | | |
| Acrylamide/Sodium | | | | | | | | | | | |
| Acryloyldimethyltaurate | | | | | | | | | | | |
| Copolymer, Isohexadecane, | | | | | | | | | | | |
| Polysorbate 80 | | | | | | | | | | | |
| Simulgel NS Hydroxyethyl | | | 2.0 | | | | | | | | |
| Acrylate/Sodium | | | | | | | | | | | |
| Acryloyldimethyl Taurate | | | | | | | | | | | |
| Copolymer, Squalane, | | | | | | | | | | | |
| Polysorbate 60 | | | | | | | | | | | |
| Sodium Benzoate Sodium | | 0.1 | | | | | | | | | |
| Benzoate | | | | | | | | | | | |
| Sodium Hydroxide 30% | | 0.5 | | | 0.46 | | | | | 0.27 | 0.93 |
| solution Aqua, Sodium | | | | | | | | | | | |
| Hydroxide | | | | | | | | | | | |
| Solubilizer PEG-40 | | | | | | | | | | 1.5 | |
| Hydrogenated castor oil, | | | | | | | | | | | |
| Trideceth-9, Propylene Glycol, | | | | | | | | | | | |
| Aqua | | | | | | | | | | | |
| Suntan accelerator PSP | | 1.5 | | | | | | | | | |
| Glycerin, Aqua, Acetyl Tyrosine, | | | | | | | | | | | |
| Riboflavin, Phenoxyethanol | | | | | | | | | | | |
| and Potassium Sorbate | | | | | | | | | | | |
| Sweet Almond Oil Prunus | | | | | | 2.5 | | | | | |
| Amygdala Dulcis Oil | | | | | | | | | | | |
| SymCalmin Butylene Glycol, | | | | | 0.5 | | | | | | |
| Pentylene Glycol, | | | | | | | | | | | |
| Hydroxyphenyl | | | | | | | | | | | |
| Propamidobenzoic Acid | | | | | | | | | | | |
| SymCare O Hexyldecanol, | | | | | | | | | | | 4.0 |
| Pentylene glycol, 4-t- | | | | | | | | | | | |
| butylcyclohexanol, Bisabolol, | | | | | | | | | | | |
| Cetylhydroxyproline | | | | | | | | | | | |
| palmitamide, Hydroxyphenyl | | | | | | | | | | | |
| propamidobenzoic acid, Stearic | | | | | | | | | | | |
| acid, Brassica campestris | | | | | | | | | | | |
| (rapeseed) sterols, Zingiber | | | | | | | | | | | |
| officinale (ginger) root extract | | | | | | | | | | | |
| SymControl Care Aqua, | | | | | 1.0 | | | | | | |
| Glycerin, Tetraselmis Suecica | | | | | | | | | | | |
| Extract | | | | | | | | | | | |
| SymDecanox HA | | | | | 2.0 | 1.0 | | 1.0 | | | 0.5 |
| Caprylic/Capric Triglyceride, | | | | | | | | | | | |
| Hydroxymethoxyphenyl | | | | | | | | | | | |
| Decanone | | | | | | | | | | | |
| SymDiol 68 1,2-Hexanediol, | | | | | | | | | | | 1.0 |
| Caprylyl glycol | | | | | | | | | | | |
| SymFinity 1298 Echinacea | | | | | | | | | | 0.06 | |
| Purpurea Extract | | | | | | | | | | | |
| SymGlucan Aqua, Glycerin, | | | | | | | | | | 5.0 | 2.0 |
| Beta-Glucan, 1,2-Hexanediol, | | | | | | | | | | | |
| Caprylyl Glycol | | | | | | | | | | | |
| SymMatrix Maltodextrin | | | | | 0.3 | | | | | | |
| (and) Rubus Fruticosus | | | | | | | | | | | |
| (Blackberry) Leaf Extract | | | | | | | | | | | |
| SymMollient PDCC | | | | | 2.0 | | | 9.0 | | | |
| Propanediol | | | | | | | | | | | |
| Dicaprylate/Caprate | | | | | | | | | | | |
| SymMollient S Cetearyl | | | | | | 3.0 | 3.0 | 6.0 | 1.0 | | 1.0 |
| Nonanoate | | | | | | | | | | | |
| SymMollient W/S Trideceth-9, | | | | | | | 3.0 | | | 1.5 | |
| PEG-5 Isononanoate, Aqua | | | | | | | | | | | |
| SymOcide PH | | | | | | | 1.0 | | | | |
| Phenoxyethanol, | | | | | | | | | | | |
| Hydroxyacetophenone, | | | | | | | | | | | |
| Caprylylglycol, Aqua | | | | | | | | | | | |
| SymOcide PS | | 0.8 | 1.0 | | | | | | | | |
| Phenoxyethanol, Decylene | | | | | | | | | | | |
| Glycol, 1,2-Hexanediol | | | | | | | | | | | |
| SymRelief 100 Bisabolol, | | | | | 0.1 | | | | | | |
| Zingiber Officinale Root Extract | | | | | | | | | | | |
| SymRepair 100 | | | | | | 2.0 | | | 1.0 | | |
| Hexyldecanol, Bisabolol, | | | | | | | | | | | |
| Cetylhydroxyproline | | | | | | | | | | | |
| Palmitamide, Stearic Acid, | | | | | | | | | | | |
| Brassica Campestris (Rapeseed) | | | | | | | | | | | |
| Sterols | | | | | | | | | | | |
| SymSave H | | | | 0.5 | 0.5 | | | | | 0.5 | 0.5 |
| Hydroxyacetophenone | | | | | | | | | | | |
| SymSitive 1609 Pentylene | | | | | | | | | | 1.0 | |
| Glycol, 4-t-Butylcyclohexanol | | | | | | | | | | | |
| SymUrban Benzylidene | | | | 0.2 | | | | | 0.5 | | |
| Dimethoxydimethylindanone | | | | | | | | | | | |
| Tapioca Pure Tapioca Starch | | | | | | | | | | | 2.0 |
| Tegin BL 315 Glycol | | | | | | | 1.5 | | | | |
| Distearate | | | | | | | | | | | |
| Texapon N70 Sodium | | | | | | | 16.0 | | | | |
| Laureth Sulfate | | | | | | | | | | | |
| Tinogard TT Pentaerythrityl | | 0.1 | | 0.1 | | | | | 0.1 | | |
| Tetra-di-t-butyl | | | | | | | | | | | |
| Hydroxyhydrocinnamate | | | | | | | | | | | |
| Tocopherol Tocopherol | | | | | | | | | | | 0.5 |
| Tocopheryl Acetate | 0.5 | | 0.3 | 0.3 | | | | | 0.5 | | |
| Tocopheryl Acetate | | | | | | | | | | | |
| Tocopheryl Linoleate | | 0.5 | | | | | | | | | |
| Tocopheryl Linoleate | | | | | | | | | | | |
| Tres BN PUHP 1108 Boron | | | | 0.25 | | | | | | | |
| Nitride | | | | | | | | | | | |
| Vegetable oil triglyceride | | | 4.0 | 3.0 | | 27.45 | | Ad 100 | | | 2.5 |
| Caprylic/Capric Triglyceride | | | | | | | | | | | |
| Water Aqua | | Ad 100 | Ad 100 | Ad 100 | Ad 100 | | Ad 100 | | | Ad 100 | Ad 100 |
| Xiameter PMX-0246 | | | | | | | | | 2.0 | | |
| Cyclohexasiloxane, | | | | | | | | | | | |
| Cyclopentasiloxane | | | | | | | | | | | |

Description of Table 39:
1=Sunless tanning dry body oil
2=Tan activating body lotion
3=Bronzing skin care
4=Tan prolonging Top to Toe Cream
5=Self-tanning gel for face
6=Dermo-protecting and bronzing shower oil
7=Oil in Cream Shower gel
8=SPF Bronzing Stick
9=Tanning Sun Protection Mist exp. SPF30, UVA/UVB Balance
10=After sun refreshing, tan prologing gel
11=Tan Prolonging Daily Tattoo Care with exp. SPF 10, UVA/UVB Balanced

TABLE 40
Cosmetic formulations 12 to 22 (amounts in parts b.w.)

| Ingredients INCI name | 12 | 13 | 14 | 15 | 16 | 17 | 18 | 19 | 20 | 21 | 22 |
|---|---|---|---|---|---|---|---|---|---|---|---|
| T-ISO SFE extract (0.9 wt- | | 2.5 | | | 1.5 | | | | | 2.0 | 4.0 |
| %) containing liquid | | | | | | | | | | | |
| mixture using vegetable | | | | | | | | | | | |
| oil triglyceride | | | | | | | | | | | |
| (Caprylic/Capric | | | | | | | | | | | |
| Triglyceride) as described | | | | | | | | | | | |
| in Example 5 | | | | | | | | | | | |
| T-ISO SFE extract (2.0 wt- | 0.2 | | | | | 1.5 | 2.0 | | 3.0 | | |
| %) containing liquid | | | | | | | | | | | |
| mixture using soybean oil | | | | | | | | | | | |
| (Glycine Soja (Soybean) | | | | | | | | | | | |
| Oil) as described in | | | | | | | | | | | |
| Example 5 | | | | | | | | | | | |
| T-ISO SFE extract (2.0 wt- | | | | 1.5 | | | | | | | |
| %) containing liquid | | | | | | | | | | | |
| mixture using | | | | | | | | | | | |
| hexyldecanol (Cetyl | | | | | | | | | | | |
| Alcohol) as described in | | | | | | | | | | | |
| Example 10 | | | | | | | | | | | |
| T-ISO SFE extract (1.5 wt- | | | 0.5 | | | | | 2.0 | | | |
| %) containing solid | | | | | | | | | | | |
| mixture using | | | | | | | | | | | |
| maltodextrin as described | | | | | | | | | | | |
| in Example 11 | | | | | | | | | | | |
| Actipone Orange Flower | | | | | 0.5 | | | | | | |
| Aqua, Propylene Glycol, | | | | | | | | | | | |
| Citrus aurantium amara | | | | | | | | | | | |
| flower extract | | | | | | | | | | | |
| Aloe Vera Gel | | | 0.2 | | | | | | | | |
| Concentrate 10/1 | | | | | | | | | | | |
| Aloe Barbadensis Leaf Juice | | | | | | | | | | | |
| Amisoft GS-11P Sodium | | | | 20.0 | | | | | | | |
| Stearoyl Glutamate, | | | | | | | | | | | |
| Sodium Cocoyl Glutamate | | | | | | | | | | | |
| Apricot Kernel Oil | | | 1.0 | | | | | | | | |
| Prunus Armeniaca Kernel | | | | | | | | | | | |
| Oil | | | | | | | | | | | |
| Beeswax Beeswax | | | | | | 0.8 | | | | | |
| Belsil ® CDM 3526 VP | | | | | | | | | | 1.0 | |
| C26-28 Alkyl Dimethicone | | | | | | | | | | | |
| Bentone Gel ® VS-5PC V | | | | | | | | | 2.5 | | |
| Cyclopentasiloxane, | | | | | | | | | | | |
| Disteardimonium | | | | | | | | | | | |
| Hectorite, Propylene | | | | | | | | | | | |
| Carbonate | | | | | | | | | | | |
| Biotive L-Arginine | | | | | | | | | | 1.5 | |
| Arginine | | | | | | | | | | | |
| Camomile CL | | | | | | | 1.0 | | | | |
| Chamomilla Recutita | | | | | | | | | | | |
| (Matricaria) Flower Water, | | | | | | | | | | | |
| Butylene Glycol, Pentylene | | | | | | | | | | | |
| Glycol | | | | | | | | | | | |
| Carbopol Ultrez 21 | 0.5 | | | | | | | | | | |
| Polymer Acrylates/C10- | | | | | | | | | | | |
| 30 Alkyl Acrylate | | | | | | | | | | | |
| Crosspolymer | | | | | | | | | | | |
| Cegosoft SBE | | | 2.0 | | | | | | | | |
| Butyrosperum Parkii | | | | | | | | | | | |
| Butter; Tocopherol | | | | | | | | | | | |
| Citric acid 10% solution | | | | | 0.45 | | 0.1 | | | | |
| Aqua, Citric acid | | | | | | | | | | | |
| Cocoa Butter Theobroma | | | | 2.0 | | | | | | | |
| Cacao (Cocoa) Seed Butter | | | | | | | | | | | |
| Coconut oil Cocos | | | | 3.3 | | | | | | | |
| Nucifera (Coconut) Oil | | | | | | | | | | | |
| Cosmedia ® SP Sodium | | | | | 1.2 | | | | | | |
| Polyacrylate | | | | | | | | | | | |
| Cosmetic Color Brown | | | | | | | | | 1.0 | | |
| Powder CI 77891, CI | | | | | | | | | | | |
| 77492, CI 77491, CI 77499 | | | | | | | | | | | |
| Covathick 2009 | | | | | | 0.5 | | | | | |
| Microcrystalline Cellulose, | | | | | | | | | | | |
| Cellulose Gum | | | | | | | | | | | |
| Crinipan PMC green | | | | | | | | 0.5 | | | |
| Propanediol caprylate | | | | | | | | | | | |
| Crodamol IPP Isopropyl | | 5.0 | | | | | 3.0 | | | | |
| Palmitate | | | | | | | | | | | |
| Cutina GMS V Glyceryl | | 1.0 | 2.0 | | | | | | | | |
| Stearate | | | | | | | | | | | |
| Cyclomethicone | | | | | | | 0.2 | | | | |
| Cyclomethicone | | | | | | | | | | | |
| Dehymuls SMO Sorbitan | | | | 0.2 | | | | | | | |
| Oleate | | | | | | | | | | | |
| Dehyquart A CA | | | | | | | | 0.5 | | | |
| Cetrimonium Chloride | | | | | | | | | | | |
| Dehyquart SP | | | | | | | | 4.0 | | | |
| Quaternium-52 | | | | | | | | | | | |
| Dermofeel PA-3 Sodium | | | 0.1 | | | | | | | | |
| Phytate; Aqua; Alcohol | | | | | | | | | | | |
| Dermofeel PS | | | 3.0 | | | | | | | | |
| Polyglyceryl-3 Stearate | | | | | | | | | | | |
| Dermofeel sensolv | | | 5.0 | 1.0 | | | | | | | |
| Isoamyl Laurate | | | | | | | | | | | |
| Dermofeel SL Sodium | | | 2.0 | | | | | | | | |
| Stearoyl Lactylate | | | | | | | | | | | |
| Dermosoft 1388 eco | | | 3.0 | | | | | | | | |
| Glycerin, Aqua, Sodium | | | | | | | | | | | |
| Levulinate, Sodium | | | | | | | | | | | |
| Anisate | | | | | | | | | | | |
| Dermosoft decalact MB | | | 0.8 | | | | | | | | |
| Sodium Caproyl/Lauroyl | | | | | | | | | | | |
| Lactylate | | | | | | | | | | | |
| Dihydroxyacetone | | 3.0 | | | | | | | | | |
| Dihydroxyacetone | | | | | | | | | | | |
| Dimethicone | | 0.5 | | | | | | 0.1 | | | |
| Dimethicone | | | | | | | | | | | |
| Dracorin CE Glyceryl | | | | | | | | 1.0 | | | |
| Stearate Citrate | | | | | | | | | | | |
| Dracorin GOC Glyceryl | | | | | | | | | | 2.5 | |
| Oleate Citrate, | | | | | | | | | | | |
| Caprylic/Capric | | | | | | | | | | | |
| Triglyceride | | | | | | | | | | | |
| Dragosantol Bisabolol, | | | | 0.1 | | | | | | | |
| Farnesol | | | | | | | | | | | |
| Dragosantol 100 | | | | | | | 0.1 | | | | |
| Bisabolol | | | | | | | | | | | |
| Dragoxat 89 Ethylhexyl | | | | | | | | | | 3.0 | |
| Isononanoate | | | | | | | | | | | |
| DryFlo ® TS Tapioca | | | | | | | | | | 1.5 | |
| Starch, | | | | | | | | | | | |
| Polymethylsilsesquioxane | | | | | | | | | | | |
| Edeta BD Disodium EDTA | 0.05 | | | | | 0.05 | | 0.1 | | 0.1 | |
| Emulgin B2 Ceteareth-20 | | | | | | 1.0 | 2.5 | | | | |
| Emulsiphos Potassium | | 2.5 | | | | | | | | | |
| Cetyl Phosphate, | | | | | | | | | | | |
| Hydrogenated Palm | | | | | | | | | | | |
| Glycerides | | | | | | | | | | | |
| Erythrulose Erythrulose | | 1.5 | | | | | | | | | |
| Ethox 4075 Glyceryl | | | | | | | | | | | 27.8 |
| Isostearate | | | | | | | | | | | |
| Caprylic/Capric Glycerides | | | | | | | | | | | |
| Eusolex ® T-AVO | | | | | | | | | | 1.0 | |
| Titanium Dioxide, Silica | | | | | | | | | | | |
| Expert Gel EG 56 BIS- | | | | | | | | | | 0.2 | |
| METHOXY PEG-13 PEG- | | | | | | | | | | | |
| 438/PPG-110 SMDI | | | | | | | | | | | |
| Copolymer | | | | | | | | | | | |
| Food Color Brown | | | | | | | | | 4.0 | | |
| E172 + E171 Powder | | | | | | | | | | | |
| Titanium Dioxides (CI | | | | | | | | | | | |
| 77891), IRON OXIDES (CI | | | | | | | | | | | |
| 77492), IRON OXIDES (CI | | | | | | | | | | | |
| 77491), IRON OXIDES (CI | | | | | | | | | | | |
| 77499) | | | | | | | | | | | |
| Fragrance Parfum | | | 0.3 | 1.0 | 0.5 | 0.8 | 0.1 | 0.5 | 1.5 | 0.5 | 0.3 |
| Frescolat Plus Menthol, | | | | | | 0.5 | | | | | |
| Menthyl Lactate | | | | | | | | | | | |
| Frescolat X-Cool | | | | | 2.0 | | | | | 0.5 | |
| Menthyl Ethylamido | | | | | | | | | | | |
| Oxalate | | | | | | | | | | | |
| Genamin ® KDMP | | | | | | 2.5 | | | | | |
| Behentrimonium chloride | | | | | | | | | | | |
| Glycerin Glycerin | | 3.0 | 2.0 | | | 1.0 | 3.0 | | | | |
| Hemp Oil Cannabis | | | | 1.0 | | | | | | | |
| Sativa Seed Oil | | | | | | | | | | | |
| Hostapon SCI-85 | | | | 30.0 | | | | | | | |
| Sodium Cocoyl Isethionate | | | | | | | | | | | |
| HydraSynol DOI | | | | 2.0 | | | | | | | |
| Isosorbide Dicaprylate | | | | | | | | | | | |
| Hydrolite 5 Pentylene | | 2.0 | | | | | | | | | 1.0 |
| Glycol | | | | | | | | | | | |
| Hydrolite 6 1,2- | 0.5 | | | 1.0 | | | 1.0 | | | | |
| Hexanediol | | | | | | | | | | | |
| Hydrolite CG Caprylyl | | | | | | 0.8 | | 0.5 | | | |
| Glycol | | | | | | | | | | | |
| Isoadipate Diisopropyl | | | | | | | | | 7.0 | | |
| Adipate | | | | | | | | | | | |
| Isodragol Triisononanoin | | | | | 5.0 | | | | | | 5.0 |
| Isopropyl Myristate | | | | | | | 0.5 | | | | |
| Isopropyl myristate | | | | | | | | | | | |
| Jojoba Oil Simmondsia | | | | | 1.5 | | | | | | |
| Chinensis Seed Oil | | | | | | | | | | | |
| Lactic acid Lactic acid | | | | | | 1.35 | | | | | |
| Lanette 16 Cetyl alcohol | | | | | | 4.0 | | | | | |
| Lanette 18 Stearyl | | | | | | 4.0 | | | | | |
| Alcohol | | | | | | | | | | | |
| Lanette O Cetearyl | | 2.5 | 1.0 | Ad 100 | | | 2.5 | | | | |
| Alcohol | | | | | | | | | | | |
| Lara Care A-200 | | | | | | | | 0.5 | | | |
| Galactoarabinan | | | | | | | | | | | |
| MelanoBronze Vitex | | | | | | | 0.5 | | | | |
| Agnus Castus Extract, | | | | | | | | | | | |
| Acetyl Tyrosine, Glycerin, | | | | | | | | | | | |
| Alcohol, Aqua | | | | | | | | | | | |
| Mineral Oil Paraffinum | | | | | | | 2.0 | | | | |
| Liquidum | | | | | | | | | | | |
| Neo-Heliopan 303 | | | | | | | | | | 10.0 | |
| Octocrylene | | | | | | | | | | | |
| Neo-Heliopan 357 Butyl | | | | | | | | | | 4.0 | |
| Methoxydibenzoylmethane | | | | | | | | | | | |
| Neo-Heliopan HMS | | | | | | | | | | 10.0 | |
| Homosalate | | | | | | | | | | | |
| Neo-Heliopan Hydro | | | | | | | | | | 1.5 | |
| Phenylbenzimidazole | | | | | | | | | | | |
| sulfonic acid | | | | | | | | | | | |
| Neo-Heliopan OS | | | | | | | | | | 5.0 | |
| Ethylhexyl Salicylate | | | | | | | | | | | |
| Olive oil Olea Europaea | | | | | | | 0.5 | | | | |
| Fruit Oil | | | | | | | | | | | |
| Oxynex ST Liquid | | | | | | | 0.3 | | 0.1 | | 0.2 |
| Diethylhexyl | | | | | | | | | | | |
| Syringylidenemalonate, | | | | | | | | | | | |
| Caprylic/Capric | | | | | | | | | | | |
| Triglyceride | | | | | | | | | | | |
| Passion Fruit oil | | | | 5.5 | | | | | | | |
| Passiflora Edulis Seed Oil | | | | | | | | | | | |
| Pemulen ™ TR-2 | | | | | | | | | | 0.25 | |
| Polymeric Emulsifier | | | | | | | | | | | |
| Acrylates/C10-30 Alkyl | | | | | | | | | | | |
| Acrylate Crosspolymer | | | | | | | | | | | |
| PCL Liquid Cetearyl | | 6.0 | | | | | | | | | |
| Ethylhexanoate, Isopropyl | | | | | | | | | | | |
| Myristate | | | | | | | | | | | |
| PCL Liquid 100 Cetearyl | | | | | | | | 0.3 | 5.5 | | 7.5 |
| Ethylhexanoate | | | | | | | | | | | |
| PCL Solid Stearyl | | | | | 3.5 | | | 3.0 | | | |
| Heptanoate, Stearyl | | | | | | | | | | | |
| Caprylate | | | | | | | | | | | |
| Phytoconcentrole Carrot | | | 1.0 | | | | | | | | |
| Helianthus Annuus Seed | | | | | | | | | | | |
| Oil, Beta-Carotene, | | | | | | | | | | | |
| Daucus Carota Sativa Root | | | | | | | | | | | |
| Extract | | | | | | | | | | | |
| Phytosqualan, veg | | | 4.0 | | | | | | | | |
| grade Squalane | | | | | | | | | | | |
| Propane-Butane | | | | | | | | | Ad 100 | | |
| Propane, Butane | | | | | | | | | | | |
| Propylene Glycol | | | | | | | | | | 5.0 | |
| Propylene Glycol | | | | | | | | | | | |
| PVP K-90 polymer PVP | 0.7 | | | | | | | | | | |
| Sensiva SC 50 | | | | | | | 0.3 | | | | |
| Ethylhexylglycerin | | | | | | | | | | | |
| Shea Butter (Organic) | | | | | | 3.0 | | | | | |
| Butyrospermum Parkii | | | | | | | | | | | |
| Butter | | | | | | | | | | | |
| SilCare ® Silicone 41M65 | | | | | | | | | | 1.0 | |
| Stearyl Dimethicone | | | | | | | | | | | |
| Silsoft ™ 034 Caprylyl | | | | | | | | | | 1.5 | |
| Methicone | | | | | | | | | | | |
| Simulgel NS | | 1.0 | | | | | | | | | |
| Hydroxyethyl | | | | | | | | | | | |
| Acrylate/Sodium | | | | | | | | | | | |
| Acryloyldimethyl Taurate | | | | | | | | | | | |
| Copolymer, Squalane, | | | | | | | | | | | |
| Polysorbate 60 | | | | | | | | | | | |
| Sisterna A10E-C Sucrose | | | | 2.5 | | | | | | | |
| Tetrastearate Triacetate | | | | | | | | | | | |
| Sodium Stearate Sodium | | | | | | | | | | | 2.8 |
| Stearate | | | | | | | | | | | |
| Sunflower Oil Helianthus | | | 2.0 | | | | | | | | |
| Annuus Seed Oil | | | | | | | | | | | |
| Super Hartolan Lanolin | | | | | | | 0.2 | | | | |
| Alcohol | | | | | | | | | | | |
| SymControl Scalp Aqua, | | | | | | 2.0 | | | | | |
| Glycerin, Mannitol, | | | | | | | | | | | |
| Tetraselmis Suecica | | | | | | | | | | | |
| Extract | | | | | | | | | | | |
| SymDecanox HA | | | | 0.5 | | | | | 1.0 | | 0.5 |
| Caprylic/Capric | | | | | | | | | | | |
| Triglyceride, | | | | | | | | | | | |
| Hydroxymethoxyphenyl | | | | | | | | | | | |
| Decanone | | | | | | | | | | | |
| SymDeo Plus Lauryl | | | | | | 0.5 | | | | | |
| alcohol, Phenoxyethanol, | | | | | | | | | | | |
| 2-benzylheptanol, | | | | | | | | | | | |
| Decylene glycol | | | | | | | | | | | |
| SymDiol 68 1,2- | | | 0.4 | | 1.2 | | 1.0 | | | | 0.6 |
| Hexanediol, Caprylyl | | | | | | | | | | | |
| glycol | | | | | | | | | | | |
| SymEffect Sun Cera | | | | | | | | | | 1.5 | |
| Alba. Sodium Stearoyl | | | | | | | | | | | |
| Lactylate | | | | | | | | | | | |
| SymFit 1617 | | | | | 0.2 | | | | | | 0.2 |
| Trimethylcyclohexy | | | | | | | | | | | |
| Butylcarbamate | | | | | | | | | | | |
| SymHair Restore | | | | | | 1.0 | | | | | |
| Glycerin, Triticum vulgare | | | | | | | | | | | |
| protein, Aqua | | | | | | | | | | | |
| SymLash 1631 Pentylene | 0.2 | | | | | | | | | | |
| Glycol, Isochrysis Galbana | | | | | | | | | | | |
| Extract | | | | | | | | | | | |
| SymLift Aqua, Trehalose, | | | 2.0 | | | | | | | | |
| Glycerin, Pentylene Glycol, | | | | | | | | | | | |
| Beta-Glucan, Hordeum | | | | | | | | | | | |
| vulgare seed extract, | | | | | | | | | | | |
| sodium hyluronate, 1,2- | | | | | | | | | | | |
| Hexanediol, Caprylyl | | | | | | | | | | | |
| Glycol | | | | | | | | | | | |
| SynLite G8 Glyceryl | | | | 0.8 | | | | 0.2 | | | |
| Caprylate | | | | | | | | | | | |
| SymMollient PDCC | | | | | | | | | 1.5 | | 10.0 |
| Propanediol | | | | | | | | | | | |
| Dicaprylate/Caprate | | | | | | | | | | | |
| SymMollient S green | | | | | | 2.0 | | | 2.0 | 2.0 | 3.0 |
| Cetearyl Nonanoate | | | | | | | | | | | |
| SymOcide PS | | 1.0 | | | 1.0 | | | 1.0 | | 1.25 | |
| Phenoxyethanol, Decylene | | | | | | | | | | | |
| Glycol, 1,2-Hexanediol | | | | | | | | | | | |
| SymOleo ® Vita7 Glycine | | | | | | 3.0 | | | | | |
| soja oil, Gossypium | | | | | | | | | | | |
| herbaceum seed oil, | | | | | | | | | | | |
| mangifera idica seed | | | | | | | | | | | |
| butter, olea europaea | | | | | | | | | | | |
| fruitoil, persea gratissima | | | | | | | | | | | |
| oil, prununs amygdalus | | | | | | | | | | | |
| dulcis (sweet almond) oil, | | | | | | | | | | | |
| Theobroma cacao seed | | | | | | | | | | | |
| butter | | | | | | | | | | | |
| SymReboot L19 | | | | | | | 0.5 | | | | |
| Maltodextrin, Lactobacillus | | | | | | | | | | | |
| Ferment | | | | | | | | | | | |
| SymSave H | 0.5 | | | | | 0.5 | | | | | 0.1 |
| Hydroxyacetophenone | | | | | | | | | | | |
| SymSitive 1609 | | | | | | | 0.5 | | | | |
| Pentylene Glycol, 4-t- | | | | | | | | | | | |
| Butylcyclohexanol | | | | | | | | | | | |
| SymSoft Scalp | | | | | | | | 1.0 | | | |
| Trideceth-9, PEG-5- | | | | | | | | | | | |
| Ethylhexanoate, Aqua, | | | | | | | | | | | |
| Hydroxyphenyl | | | | | | | | | | | |
| Propamidobenzoic Acid | | | | | | | | | | | |
| SymSol ® PF-3 Aqua, | | | | | | | | | | 3.0 | |
| Pentylene Glycol, Sodium | | | | | | | | | | | |
| Lauryl Sulfoacetate, | | | | | | | | | | | |
| Sodium Oleoyl | | | | | | | | | | | |
| Sarcosinate, Sodium | | | | | | | | | | | |
| Chloride, Sodium Oleate | | | | | | | | | | | |
| SymVital AR 3040 | | | | | | | | | | 0.1 | |
| Zinbiber officinale root | | | | | | | | | | | |
| extract | | | | | | | | | | | |
| Tagat L2 PEG-20 Glyceryl | | | | | | | 2.5 | | | | |
| Laurate | | | | | | | | | | | |
| Talco Talc | | | | | | | | | 2.0 | | |
| Tego ® Amid S18 | | | | | | 3.0 | | | | | |
| Stearamidopropyl | | | | | | | | | | | |
| Dimethylamine | | | | | | | | | | | |
| Tego Feel Green | | | | 1.0 | | | | | | | |
| Cellulose | | | | | | | | | | | |
| Thermolat Vanillyl Butyl | | | | | 1.0 | | | | | | |
| Ether, 1,2-Hexanediol, | | | | | | | | | | | |
| Caprylyl Glycol | | | | | | | | | | | |
| Tinogard TT | | 0.1 | | | | | | | | | |
| Pentaerythrityl | | | | | | | | | | | |
| Tetra-di-t-butyl | | | | | | | | | | | |
| Hydroxyhydrocinnamate | | | | | | | | | | | |
| Tocopherol Tocopherol | | | | 0.2 | | | | 0.05 | | | 0.1 |
| Tocopheryl Acetate | | 0.5 | | | 0.3 | | | | | 0.5 | |
| Tocopheryl Acetate | | | | | | | | | | | |
| Triethanolamine 90% | 0.45 | | | | | | | | | | |
| Care Triethanolamine | | | | | | | | | | | |
| Varisoft EQ65 | | | | | | 4.0 | | | | | |
| Distearoylethyl Dimonium | | | | | | | | | | | |
| Chloride, Ceterayl Alcohol | | | | | | | | | | | |
| Veegum Ultra Granules | | | 0.5 | | | | | | | | |
| Magnesium Aluminium | | | | | | | | | | | |
| Silicate | | | | | | | | | | | |
| Vegetable oil | | 5.5 | | | | | | 0.8 | | | Ad 100 |
| triglyceride | | | | | | | | | | | |
| Caprylic/Capric | | | | | | | | | | | |
| Triglyceride | | | | | | | | | | | |
| Water Aqua | Ad 100 | Ad 100 | Ad 100 | | Ad 100 | Ad 100 | Ad 100 | Ad 100 | | Ad 100 | |
| Xanthan Gum Xanthan | | 0.3 | 0.5 | | | | | | | | |
| Gum | | | | | | | | | | | |
| Xiameter ® PMX-1503 | | | | | | | | | | 1.0 | |
| Fluid Dimethicone, | | | | | | | | | | | |
| Dimethiconol | | | | | | | | | | | |

Description of Table 40:

12=Eyelash and Eyebrow Serum
13=Self Tanning body Lotion
14=Bronzing Night Cream (w/o)
15=All in one bar—Shampoo—body
16=Cold & Hot Firming Gel with bronzing effect
17=Color intensifying revitalizing conditioner, leave-on
18=Bronzing Soft Wet Wipes
19=Hair toning, scalp soothing hair conditioner, rinse-off
20=Tanning legs spray (dark brown)
21=3 in 1 bronzing sunscreen spray
22=Solid Tan Prolonging Body Oil

TABLE 41
Sun protective bronzing body oil

| Ingredients | INCI | wt. % | wt. % |
|---|---|---|---|
| T-ISO SFE extract (0.9 wt-%) containing liquid | | 1.5 | 4.0 |
| mixture using vegetable oil triglyceride | | | |
| (Caprylic/Capric Triglyceride) | | | |
| as described in Example 5 | | | |
| Sweet Almond Oil | Prunus Amygdala Dulcis Oil | 7.5 | 4.5 |
| Vegetable oil | Caprylic/Capric | 6.0 | 7.5 |
| triglyceride | Triglyceride | | |
| Jojoba Oil | Simmondsia Chinensis Seed | 15.0 | 15.0 |
| | Oil | | |
| Amisol Trio | Phospholipids, Glycine Soja | 2.0 | 2.0 |
| | Oil, Glycolipids, Glycine Soja | | |
| | Sterols | | |
| Dermofeel TC-7 | Triheptanoin | 8.9 | 8.9 |
| Schercemol CO Ester | Cetyl Ethylhexanoate | Ad 100 | Ad 100 |
| Neo-Heliopan 303 | Octocrylene | 5.0 | 5.0 |
| Neo-Heliopan 357 | Butyl | 3.1 | 3.1 |
| | Methoxydibenzoylmethane | | |
| Neo-Heliopan AV | Ethylhexyl Methoxycinnamate | 5.0 | 5.0 |
| Neo-Heliopan OS | Ethylhexyl Salicylate | 5.0 | 5.0 |
| Fragrance | Parfum | 0.3 | 0.8 |
| Vitapherole E-1000 | Tocopherol, Helianthus | 0.2 | 0.1 |
| | Annuus Seed Oil | | |
| MelinOil | Isopropyl Palmitate, Lecithin, | 5.0 | — |
| | Water, Acetyl Hexapeptide-1 | | |

TABLE 42
Self-tanning o/w cream with sun screens

| Ingredients | INCI | wt. % | wt. % | wt. % |
|---|---|---|---|---|
| T-ISO SFE extract (0.9 wt-%) containing liquid | | — | 2.5 | 3.5 |
| mixture using vegetable oil triglyceride | | | | |
| (Caprylic/Capric Triglyceride) | | | | |
| as described in Example 5 | | | | |
| T-ISO SFE extract (2.0 wt-%) containing liquid | | 1.0 | — | — |
| mixture using hexyldecanol (Cetyl Alcohol) | | | | |
| as described in Example 10 | | | | |
| Vegetable oil triglyceride | Caprylic/Capric Triglyceride | 2.5 | — | — |
| Lanette 16 | Cetyl Alcohol | — | 1.0 | 1.0 |
| Lexemul 561 MB | Glyceryl Stearate, PEG-100 | 5.0 | 5.0 | 5.0 |
| | Stearate | | | |
| Stearic acid | Stearic acid | 2.0 | 2.0 | 2.0 |
| Cyclomethicone | Cyclomethicone | 2.0 | 2.0 | 2.0 |
| Dimethicone | Dimethicone | 4.0 | 4.0 | 4.0 |
| Jojoba Oil | Simmondsia Chinensis Seed Oil | 1.0 | — | — |
| Avocado Oil CA | Persea Gratissima Oil | — | 1.0 | — |
| Isopropyl Palmitate | Isopropyl palmitate | 3.5 | 3.5 | 3.5 |
| Neo-Heliopan AV | Ethylhexyl Methoxycinnamate | 2.0 | — | — |
| Neo-Heliopan E1000 | Isoamyl p-Methoxycinnamate | — | — | 2.0 |
| Neo-Heliopan Flat | Homosalate, Octocrylene, Bis- | — | 2.0 | — |
| | Ethylhexyloxyphenol | | | |
| | Methoxyphenyl Triazine, Butyl | | | |
| | Methoxydibenzoylmethane, | | | |
| | Ethylhexyl Salicylate | | | |
| Neo-Heliopan 357 | Butyl | 1.0 | 1.0 | 1.0 |
| | Methoxydibenzoylmethane | | | |
| Tocopheryl Acetate | Tocopheryl Acetate | 0.5 | 0.3 | — |
| Water | Aqua | Ad 100 | Ad 100 | Ad 100 |
| Euxyl PE9010 | Phenoxyethanol, | 0.8 | — | 0.8 |
| | Ethylhexylglycerin | | | |
| SymOcide PH | Phenoxyethanol, | — | 1.0 | — |
| | Hydroxyacetophenone, | | | |
| | Caprylylglycol, Aqua | | | |
| Glycerin | Glycerin | 2.0 | 2.0 | 2.0 |
| Butylene Glycol | Butylene Glycol | 4.0 | 4.0 | 4.0 |
| Dimethyl Isosorbide | Dimethyl Isosorbide | 2.5 | 2.5 | 2.5 |
| Erythrulose | Erythrulose | 5.0 | 3.0 | — |
| Dihydroxyacetone | Dihydroxyacetone | — | 2.0 | 5.0 |
| Troxerutin | Troxerutin | 2.5 | 2.5 | — |

TABLE 43
Bronzing emulsion

| Ingredients | INCI | wt. % | wt. % |
|---|---|---|---|
| T-ISO SFE extract (0.9 wt-%) containing liquid | | — | 3.0 |
| mixture using vegetable oil triglyceride | | | |
| (Caprylic/Capric Triglyceride) | | | |
| as described in Example 5 | | | |
| T-ISO SFE extract (2.0 wt-%) containing | | 2.0 | — |
| liquid mixture using soybean oil | | | |
| (Glycine Soja (Soybean) Oil) | | | |
| as described in Example 5 | | | |
| RonaCare Bronzyl | Dihydroxy Methylchromonyl | 0.2 | 0.1 |
| | Palmitate | | |
| Myritol 331 | Cocoglycerides | 2.0 | 1.0 |
| Tegosoft TN | C12-15 Alkyl Benzoate | 8.0 | 8.0 |
| Cetiol B | Dibutyl Adipate | 5.0 | 5.0 |
| X-Trend 226 | Phenylethyl bezoate | 4.0 | 4.0 |
| Carbopol Ultrez 10 | Carbomer | 0.2 | 0.2 |
| SLM 2026 | Aqua, Caprylic/Capric | 10.0 | — |
| | Triglyceride, Hydrogenated | | |
| | Phosphatidylcholine, | | |
| | Pentylene | | |
| | Glycol, Glycerin, | | |
| | Butyrospermum Parkii Butter, | | |
| | Squalane, Ceramide NP | | |
| Aloe Vera Gel | Aloe Barbadensis Leaf Juice | — | 0.5 |
| Concentrate 10/1 | | | |
| Erythrulose | Erythrulose | — | 3.0 |
| Propylene Glycol | Propylene Glycol | 5.0 | 5.0 |
| Water | Aqua | Ad 100 | Ad 100 |
| Xanthan Gum | Xanthan Gum | 0.05 | 0.05 |
| Sodium Hydroxide | Aqua, Sodium Hydroxide | 0.2 | 0.2 |
| 10% solution | | | |
| RonaCare | Aqua, Alcohol, Lecithin, Ectoin, | 2.0 | — |
| Cyclopeptide-5 | Cyclotetrapeptide-24 | | |
| | Aminocyclohexane Carboxylate | | |
| SymSitive 1609 | Pentylene Glycol, 4-t- | — | 0.5 |
| | Butylcyclohexanol | | |
| SymLift | Aqua, Trehalose, Glycerin, | — | 3.0 |
| | Pentylene Glycol, Beta-Glucan, | | |
| | Hordeum vulgare seed extract, | | |
| | sodium hyluronate, 1,2- | | |
| | Hexanediol, Caprylyl Glycol | | |
| SymOcide PS | Phenoxyethanol, Decylene | — | 1.0 |
| | Glycol, 1,2-Hexanediol | | |
| Euxyl PE9010 | Phenoxyethanol, | 0.8 | — |
| | Ethylhexylglycerin | | |
| Fragrance | Parfum | 0.3 | 0.5 |

TABLE 44
Self-tanning gel

| Ingredients | INCI | wt. % | wt. % |
|---|---|---|---|
| T-ISO SFE extract (0.9 wt-%) containing liquid | | — | 1.0 |
| mixture using vegetable oil triglyceride | | | |
| (Caprylic/Capric Triglyceride) | | | |
| as described in Example 5 | | | |
| T-ISO SFE extract (1.5 wt-%) containing | | 0.8 | — |
| solid mixture using maltodextrin | | | |
| as described in Example 11 | | | |
| Dihydroxyacetone | Dihydroxyacetone | 4.0 | 3.0 |
| Erythrulose | Erythrulose | — | 2.0 |
| Water | Aqua | Ad 100 | Ad 100 |
| Proylene Glycol | Proylene Glycol | 10.0 | 10.0 |
| Glycerin | Glycerin | 22.5 | 22.5 |
| Gelaid CPE | Dimethicone, | 25.0 | 25.0 |
| | Dimethicone/Vinyl | | |
| | Dimethicone Crosspolymer, | | |
| | Hydroxyethyl | | |
| | Acrylate/Sodium | | |
| | Acryloyldimethyl Taurate | | |
| | Copolymer | | |
| Euxyl PE9010 | Phenoxyethanol, | 1.0 | 1.0 |
| | Ethylhexylglycerin | | |
| Fragrance | Parfum | 0.3 | 0.5 |

| | | |
|---|---|---|
| Citric acid 50% solution | Aqua, Citric acid | qs to pH 3, 5-4 |
| Glycerin | Glycerin | qs to clear |
| | | system |

TABLE 45
Sun protecting self-tanning cream

| Ingredients | INCI | wt. % | wt. % |
|---|---|---|---|
| T-ISO SFE extract (0.9 wt-%) containing liquid | | — | 2.0 |
| mixture using vegetable oil triglyceride | | | |
| (Caprylic/Capric Triglyceride) | | | |
| as described in Example 5 | | | |
| T-ISO SFE extract (2.0 wt-%) containing | | 2.0 | |
| liquid mixture using soybean oil | | | |
| (Glycine Soja (Soybean) Oil) | | | |
| as described in Example 5 | | | |
| Dihydroxyacetone | Dihydroxyacetone | — | 1.0 |
| Erythrulose | Erythrulose | 4.0 | 3.0 |
| Water | Aqua | Ad 100 | Ad 100 |
| Buffer pH = 5 | Sodium Acetate, Acetic Acid | 0.3 | 0.3 |
| Amigel | Sclerotium Gum | 0.8 | 0.8 |
| Xanthan Gum | Xanthan Gum | 0.4 | 0.4 |
| Montanov 202 | Arachidyl Alcohol, Behenyl | 5.5 | 5.5 |
| | Alcohol, Arachidyl Glucoside | | |
| Dimethicone | Dimethicone | 3.0 | 3.0 |
| Apricot Kernel Oil | Prunus Armeniaca Kernel Oil | 2.0 | 3.0 |
| Neo-Heliopan AV | Ethylhexyl Methoxycinnamate | 2.0 | — |
| Neo-Heliopan BB | Benzophenone-3 | 2.0 | 2.0 |
| Neo-Heliopan E1000 | Isoamyl p-Methoxycinnamate | — | 2.0 |
| Butylene Glycol | Butylene Glycol | 7.7 | 7.7 |
| Fragrance | Parfum | 0.2 | 0.8 |
| Tyr-Ol | Oleoyl tyrosine, Butylene | 1.0 | 1.0 |
| | Glycol, Oleic acid | | |
| SymDecanox HA | Caprylic/Capric Triglyceride, | 1.0 | — |
| | Hydroxymethoxyphenyl | | |
| | Decanone | | |
| SymOcide PS | Phenoxyethanol, Decylene | 1.0 | — |
| | Glycol, 1,2-Hexanediol | | |
| SymOcide PH | Phenoxyethanol, | — | 1.0 |
| | Hydroxyacetophenone, | | |
| | Caprylylglycol, Aqua | | |

1. A supercritical fluid CO2 extract of Isochrysis sp. and/or Tisochrysis sp., comprising
(a) not more than 1.5% b.w. of carotenoids,
(b) not more than 2.0% b.w. of chlorophylls, and
(c) at least 15% b.w. of free fatty acids,
each weight percentage amount based on the total weight of the extract,
wherein the extract has a gardner color value of not more than 9 at an extract concentration of 0.018% in oil.

2. The extract according to claim 1, wherein the extract comprises not more than 1.0% b.w. of carotenoids, based on the total weight of the extract.

3. The extract according to claim 1, wherein the extract comprises not more than 0.5% b.w. of carotenoids, based on the total weight of the extract.

4. The extract according to claim 1, wherein the extract comprises not more than 1.0% b.w. of chlorophylls, based on the total weight of the extract.

5. The extract according to claim 1, wherein the extract comprises not more than 0.5% b.w. of chlorophylls, based on the total weight of the extract.

6. The extract according to claim 1, wherein the extract comprises at least 20% b.w. of free fatty acids, based on the total weight of the extract.

7. The extract according to claim 1, wherein the extract further comprises not more than 2.0% b.w. of phospholipids not more than 2.0% b.w. of glycolipids and an ash content of not more than 0.5% b.w., each weight percentage amount based on the total weight of the extract.

8. The extract according to claim 1, wherein the extract comprises not more than 27% b.w. of long chain alkenones, based on the total weight of the extract.

9. A method for obtaining the supercritical fluid CO2 extract according to claim 1, comprising the steps of extracting cell material of Isochrysis sp. and/or Tisochrysis sp., preferably Tahitian Isochrysis with supercritical CO2, wherein the extraction comprises
a) exposition of the cell material to the supercritical CO2 for up to 24 h, preferably for up to 8 h at a temperature of 30 to 70° C. and a pressure of 150 to 290 bar.

10. The method according to claim 9, wherein the cell material of Isochrysis sp. and/or Tisochrysis sp is obtained by a method comprising the steps of:
(a) Cultivating Isochrysis sp. cells and/or preferably of Tahitian Isochrysis cells,
(b) Harvesting the cells to obtain completely or substantially intact cell material,
(c) Optionally washing the cell material of step (b) once or multiple times, to obtain washed, substantially or completely intact cell material,
(d) Optionally freeze-drying the cell material of step (b) and/or step (c), or
(e) Optionally spray-drying the cell material of step (b) and/or step (c), or
(f) Optionally thermal drying the cell material of step (b) and/or step (c), or
(g) Optionally vacuum drying the cell material of step (b) and/or step (c), and
(h) Optionally milling the cell material of step (b) and/or step (c) and/or step (d) and/or step (e) and/or step (f) and/or step (g).

11. A method comprising using the supercritical fluid CO2 extract of Isochrysis sp. and/or Tisochrysis sp according to claim 1 for influencing or modifying human skin and/or human hair,
for increasing pigmentation of human skin and/or pigmentation of human hair, and/or
for reducing oxidative stress induced changes when applied to the skin, and/or
for increasing skin hydration, and/or
for improving the cutaneous barrier properties of the skin.

12. The method according to claim 11, wherein the oxidative stress is UV-induced oxidative stress.

13. The method according to claim 11, wherein the oxidative stress is Maillard reaction induced oxidative stress.

14. A cosmetic composition comprising the supercritical fluid CO2 extract of Isochrysis sp. and/or Tisochrysis sp according to claim 1 and at least one further ingredient selected from the group consisting of:
a) tanner,
b) skin moisturizing agents,
c) stabilizer,
d) antioxidants,
e) UV filters,
and mixtures thereof.

15. The cosmetic composition according to claim 14, wherein the tanner is selected from dihydroxyacetone and/or erythulose.
